(19) United States
(12) Patent Application Publication
(10) Pub. No.: US 2023/0212136 A1
(43) Pub. Date: Jul. 6, 2023

(54) CURABLE RESIN COMPOSITION, CURED PRODUCT, DIFFRACTIVE OPTICAL ELEMENT, AND MULTILAYER DIFFRACTIVE OPTICAL ELEMENT

(71) Applicant: FUJIFILM Corporation, Tokyo (JP)

(72) Inventors: Kosuke CHIBA, Ashigarakami-gun (JP); Naozumi SHIRAIWA, Ashigarakami-gun (JP); Takafumi NAKAYAMA, Ashigarakami-gun (JP); Naoyuki MOROOKA, Ashigarakami-gun (JP)

(73) Assignee: FUJIFILM Corporation, Tokyo (JP)

(21) Appl. No.: 18/091,139

(22) Filed: Dec. 29, 2022

Related U.S. Application Data
(63) Continuation of application No. PCT/JP21/24795, filed on Jun. 30, 2021.

(30) Foreign Application Priority Data
Jun. 30, 2020 (JP) 2020-113429

(51) Int. Cl. C07D 339/06; C01G 19/02; G02B 27/42; G02B 1/04

(52) U.S. Cl. CPC C07D 339/06; C01G 19/02; G02B 1/041; G02B 27/4211; C01P 2004/64

(57) ABSTRACT

Provided are a curable resin composition including a near-ultraviolet light-absorbing organic compound, indium tin oxide particles, and a polymer having a constitutional unit represented by General Formula (P) and having an acidic group at one terminal, in which the near-ultraviolet light-absorbing organic compound is a compound that has a maximal value at 300 to 400 nm in an absorption spectrum in a wavelength region of 300 to 800 nm and does not substantially absorb light at a wavelength of 410 to 800 nm; a cured product formed of the curable resin composition; a diffractive optical element; and a multilayer diffractive optical element.

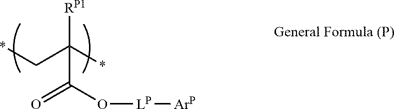

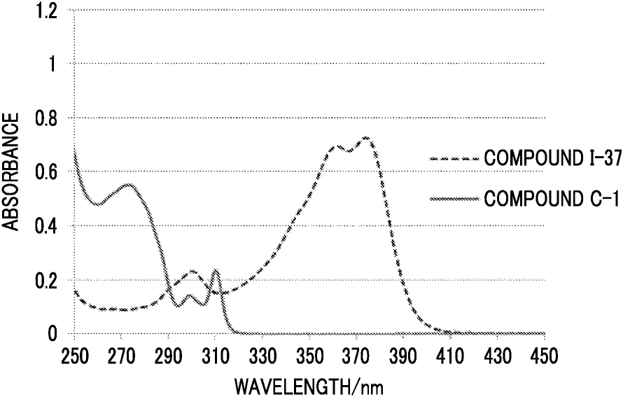

Ar^{P} represents an aryl group and L^{P} and R^{P1} represent a specific group.

### CROSS-REFERENCE TO RELATED APPLICATIONS

This application is a Continuation of PCT International Application No. PCT/JP2021/024795 filed on Jun. 30, 2021, which claims priority under 35 U.S.C. § 119 (a) to Japanese Patent Application No. 2020-113429 filed in Japan on Jun. 30, 2020. Each of the above applications is hereby expressly incorporated by reference, in its entirety, into the present application.

### BACKGROUND OF THE INVENTION

### 1. Field of the Invention

The present invention relates to a curable resin composition.

The present invention also relates to a cured product obtained using the curable resin composition, a diffractive optical element, and a multilayer diffractive optical element.

### 2. Description of the Related Art

By using a diffractive optical element, it is possible to obtain a lens which has a shorter focal length as the wavelength is longer, and exhibits chromatic aberration opposite to that of a refractive lens in the related art. Unlike the refractive lens requiring a plurality of lenses for correcting chromatic aberration, chromatic aberration can be corrected by changing the period of a diffraction structure of a lens, therefore a more compact and high-performance lens unit can be designed by using the diffractive optical element.

In a multilayer diffractive optical element having a configuration in which diffractive optical elements formed of two different materials are in contact with each other on lattice planes thereof, by forming one diffractive optical element with a material having a relatively high refractive index and high Abbe number, and forming the other diffractive optical element with a material having a relatively low refractive index and low Abbe number, it is possible to suppress the occurrence of flare in the lens, and the like, and sufficiently utilize a chromatic aberration reducing effect. In this case, in a case where the two diffractive optical elements have optical characteristics in which the difference in refractive index between the two diffractive optical elements is larger at a longer wavelength, the chromatic aberration reducing effect can be obtained in a wide wavelength range.

In recent years, in order to obtain, as described above, the chromatic aberration reducing effect in a wide wavelength range, it has been proposed to add indium tin oxide (ITO) particles to a low Abbe number diffractive optical element in the multilayer diffractive optical element. For example, JP2006-220689A discloses, as a curable resin composition for producing a diffractive optical element, a curable resin composition in which ITO fine particles are dispersed in a resin containing a photopolymerization initiator, a dispersant, and a mixture of two or more acryloyl groups, methacryloyl groups, or vinyl groups, or unsaturated ethylene groups thereof.

### SUMMARY OF THE INVENTION

In the technique disclosed in JP2006-220689A, by adding ITO particles, the refractive index of the cured product obtained from the curable resin composition in a near-infrared wavelength region is lowered, and the chromatic aberration reducing effect is improved. However, from studies by the present inventors, in an optical system which uses light in the near-infrared wavelength region, it has been found that the decrease in transmittance in the near-infrared wavelength region due to the addition of ITO particles is a problem. In addition, it has also been found that it is difficult to realize a desired low Abbe number by reducing a blending amount of the ITO particles in order to increase transmittance in the near-infrared wavelength region.

In order to deal with the above-described problems, the present inventors have made extensive studies. By adding a near-ultraviolet light-absorbing organic compound to the curable resin composition, it is possible to improve a refractive index of the obtained cured product on the short wavelength side and adjust a wavelength dependence of the refractive index. As a result, it has been found that a desired low Abbe number can be realized while increasing the transmittance in the near-infrared wavelength region by suppressing the blending amount of the ITO particles.

However, affinity between the near-ultraviolet light-absorbing organic compound and the ITO particles is low, and the curable resin composition containing the near-ultraviolet light-absorbing organic compound and the ITO particles has limitations in improving dispersion stability even in a case where a dispersant is blended. Therefore, a new problem has been found in that it is difficult to maintain the dispersion stability of the curable resin composition over a long period of time.

An object of the present invention is to provide a curable resin composition which contains ITO particles and a near-ultraviolet light-absorbing organic compound, and is excellent in medium- to long-term dispersion stability. Another object of the present invention is to provide a cured product obtained from the curable resin composition, and a diffractive optical element and a multilayer diffractive optical element including the cured product.

In view of the above-described problems, the present inventors have conducted intensive studies. As a result, in order to enhance the dispersion stability of the curable resin composition by blending a polymer dispersant to the curable resin composition containing the ITO particles and the near-ultraviolet light-absorbing organic compound, by introducing an acidic group as an adsorptive group to the ITO particles to one terminal of the polymer main chain of this polymer dispersant, and by introducing, as a constituent component of this polymer dispersant, a constitutional unit derived from a monomer having a structure in which a (meth)acryloyl group and a benzene ring are bonded directly or through a linking group, it has been found that the dispersion stability of the curable resin composition can be sufficiently enhanced over the medium to long term. The present invention has been completed by further repeating studies on the basis of the above-described finding.

That is, specific methods for achieving the above-described object are as follows.

[1] A curable resin composition comprising:
a near-ultraviolet light-absorbing organic compound;
indium tin oxide particles; and
a polymer having a constitutional unit represented by General Formula (P) and having an acidic group at one terminal,

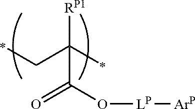

in the formula, L^{p} represents a single bond or a divalent linking group, Ar^{P} represents an aryl group, R^{P1} represents a hydrogen atom or a methyl group, where Ar^{P} does not include the acidic group, and * represents a bonding portion,
in which, in the near-ultraviolet light-absorbing organic compound, in a case where an absorbance is measured from a wavelength of 800 nm toward a short wavelength side, a wavelength at which a maximal value is first exhibited is present at 300 to 400 nm, and
in a case where an absorbance at a wavelength of λ nm is defined as A_{λ}, relationships of Expression I to III are satisfied,
$$\left({\text{A}}_{λ max} \text{-} {\text{A}}_{410}\right) / {\text{A}}_{λ max} \geq 0.97$$
$$1.00 \geq \left({\text{A}}_{λ max} \text{-} {\text{A}}_{410}\right) / \left({\text{A}}_{λ max} \text{-} {\text{A}}_{430}\right) \geq 0.97$$
$$\left({\text{A}}_{λ max} \text{-} {\text{A}}_{410}\right) / \left(410 \text{-} λ \text{max}\right) \geq 0.005$$
in the expressions, A_{λmax} indicates a maximum absorbance at 300 to 400 nm.

The curable resin composition according to [1],

in which the acidic group is selected from a carboxy group, a phosphono group, a phosphonooxy group, a hydrohydroxyphosphoryl group, a sulfino group, a sulfo group, or a sulfanyl group.

The curable resin composition according to [1] or [2],

in which the acidic group is a carboxy group.

The curable resin composition according to [3],
in which the polymer has, as a structural portion including the acidic group, a structural portion represented by General Formula (PA1) at one terminal of a polymer chain,

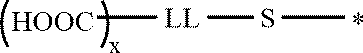

in the formula, LL represents a single bond or an (x+1)-valent linking group, where x represents an integer of 1 to 8, and * represents a bonding portion.

The curable resin composition according to any one of [1] to [4],

in which a weight-average molecular weight of the polymer is 1000 to 20000, and an acid value of the polymer is 2.0 mgKOH/g or more and less than 100 mgKOH/g.

The curable resin composition according to any one of [1] to [5],

in which L^{P} in General Formula (P) is a single bond, —CH₂—, —CH₂O—, or —CH₂CH₂O—.

The curable resin composition according to any one of [1] to [6],

in which a proportion of the constitutional unit represented by General Formula (P) to all constitutional units constituting the polymer is 10 mol% or more.

[8] The curable resin composition according to any one of [1] to [7],
in which the near-ultraviolet light-absorbing organic compound is at least one of Compounds 1 to 3,
Compound 1:

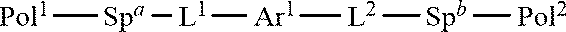

in the formula, Ar¹ represents an aromatic ring group represented by any of General Formula (2-1), ..., or (2-4),
L¹ and L² represent a single bond, —O—, —S—, —C(═O)—, —OC(═O)—, —C(═O)O—, —OC(═O)O—, —NR¹⁰¹C(═O)—, —C(═O)NR¹⁰²—, —OC(═O)NR¹⁰³—, —NR¹⁰⁴C(═O)O—, —SC(═O)—, or —C(═O)S—, R¹⁰¹ to R¹⁰⁴ represent -Sp^{c}-Pol³,
Sp^{a} represents a linking group having a shortest atom number of 2 or more and linking Pol¹ and L¹, Sp^{b} represents a linking group having the shortest atom number of 2 or more and linking Pol² and L², Sp^{c} represents a single bond or a divalent linking group, and
Pol¹ to Pol³ represent a hydrogen atom or a polymerizable group, in which at least one of Pol¹ or Pol² represents a polymerizable group, where a linking portion of Sp^{a} to L¹ and a linking portion of Sp^{b} to L² are both —CH₂—, and a linking portion of Sp^{a} to Pol¹, a linking portion of Sp^{b} to Pol², and a linking portion of Sp^{c} to Pol³ are all a carbon atom,

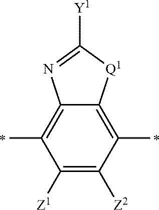

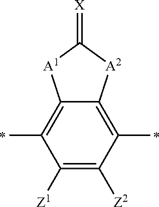

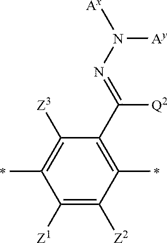

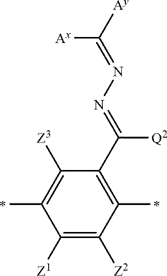

in the formulae, Q¹ represents —S—, —O—, or >NR¹¹, and R¹¹ represents a hydrogen atom or an alkyl group having 1 to 6 carbon atoms,
Y¹ represents an alkyl group having 1 to 6 carbon atoms, an aromatic hydrocarbon group having 6 to 12 carbon atoms, or an aromatic heterocyclic group having 3 to 12 carbon atoms,
Z¹, Z², and Z³ represent a hydrogen atom, an aliphatic hydrocarbon group having 1 to 20 carbon atoms, an alkoxy group having 1 to 20 carbon atoms, an alicyclic hydrocarbon group having 3 to 20 carbon atoms, an aromatic hydrocarbon group having 6 to 20 carbon atoms, a halogen atom, a cyano group, a nitro group, —NR¹²R¹³, or —SR¹², Z¹ and Z² may be bonded to each other to form an aromatic hydrocarbon ring or an aromatic heterocyclic ring, R¹² and R¹³ represent a hydrogen atom or an alkyl group having 1 to 6 carbon atoms,
A¹ and A² represent —O—, >NR²¹, —S—, or >C(═O), and R²¹ represents a hydrogen atom or a substituent,
X represents ═O, ═S, a carbon atom to which a hydrogen atom or a substituent is bonded, or a nitrogen atom to which a hydrogen atom or a substituent is bonded,
A^{x} represents an organic group having 1 to 30 carbon atoms, which has at least one aromatic ring selected from an aromatic hydrocarbon ring or an aromatic heterocyclic ring, A^{y} represents a hydrogen atom, an alkyl group having 1 to 6 carbon atoms, or an organic group having 1 to 30 carbon atoms, which has at least one aromatic ring selected from an aromatic hydrocarbon ring or an aromatic heterocyclic ring, and A^{x} and A^{y} may be bonded to each other to form a ring,
Q² represents a hydrogen atom or an alkyl group having 1 to 6 carbon atoms, and * represents a bonding position with L¹ or L²,
Compound 2:

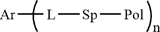

in the formula, Ar represents a group represented by General Formula (A1),
L represents a single bond, —O—, —S—, —C(═O)—, —OC(═O)—, —C(═O)O—, —OC(═O)O—, —NR³⁰¹C(═O)—, —C(═O)NR³⁰²—, —OC(═O)NR³⁰³—, —NR³⁰⁴C(═O)O—, —SC(═O)—, or —C(═O)S—,
R³⁰¹ to R³⁰⁴ represent -Sp^{d}-Pol⁴,
Sp and Sp^{d} represent a single bond or a divalent linking group, and Pol and Pol⁴ represent a hydrogen atom or a polymerizable group, and
n is an integer of 1 or 2,
where the compound represented by General Formula (A) has at least one polymerizable group,

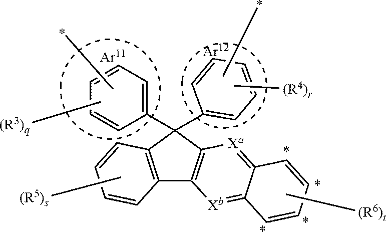

in the formula, Ar¹¹ and Ar¹² represent an aromatic hydrocarbon group including a benzene ring surrounded by a broken line or an aromatic heterocyclic group including a benzene ring surrounded by a broken line as one of rings constituting a fused ring,
X^{a} and X^{b} represent a nitrogen atom or CH, CH at a position of # may be substituted by a nitrogen atom,
R³ to R⁶ represent a substituent, q, r, s, and t are an integer of 0 to 4, and * represents a bonding position with Pol—Sp—L—,
Compound 3:

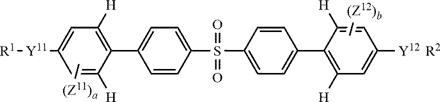

in the formula, a and b are an integer of 1 or 2, Y¹¹ and Y¹² represent —S— or —O—, R¹ and R² represent a hydrogen atom, a methyl group, or an ethyl group, and Z¹¹ and Z¹² represent a methyl group or an ethyl group having a substituent represented by General Formula (Z),

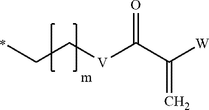

in the formula, m is an integer of 0 or 1, W represents a hydrogen atom or a methyl group, and V represents —O—CₙH₂ₙ—O—**, —S—CₙH₂ₙ—S—**, or —S—CₙH₂ₙ—O—**, where ** represents a bonding site with a (meth)acryloyl group, and n is an integer of 2 to 4, where at least one hydrogen atom in -CₙH₂ₙ- is substituted by a methyl group.

[9] The curable resin composition according to any one of [1] to [8],

in which a content of the polymer is 5 to 50 parts by mass with respect to 100 parts by mass of a content of the indium tin oxide particles.

The curable resin composition according to any one of [1] to [9],

in which a content of the indium tin oxide particles in the curable resin composition is 10% to 60% by mass.

The curable resin composition according to any one of [1] to [10],

in which a particle diameter of the indium tin oxide particles is 5 to 50 nm.

The curable resin composition according to any one of [1] to [11], further comprising:

a monofunctional or bi- or higher functional (meth)acrylate monomer compound.

The curable resin composition according to any one of [1] to [12], further comprising:

a polymerization initiator.

A cured product of the curable resin composition according to any one of [1] to [13].

A diffractive optical element comprising:
the cured product according to [14],
in which the diffractive optical element includes a surface having a diffraction grating shape and formed of the cured product.

A multilayer diffractive optical element comprising:
a first diffractive optical element; and
a second diffractive optical element,
in which the first diffractive optical element is the diffractive optical element according to [15], and
a surface of the first diffractive optical element, which has a diffraction grating shape, and a surface of the second diffractive optical element, which has a diffraction grating shape, face each other.

In the present invention, the expression of a compound and a substituent is used to include the compound itself and the substituent itself, a salt thereof, and an ion thereof. For example, a carboxy group or the like may have an ionic structure in which a hydrogen atom is dissociated, or may have a salt structure. That is, in the present invention, the “carboxy group” is used in the sense of including a carboxylic acid ion or a salt thereof. This also applies to other acidic groups. A monovalent or polyvalent cation in forming the above-described salt structure is not particularly limited, and examples thereof include inorganic cations and organic cations. In addition, specific examples thereof include alkali metal cations such as Na⁺, Li⁺, and K⁺, alkaline earth metal cations such as Mg²⁺, Ca²⁺, and Ba²⁺, and organic ammonium cations such as a trialkylammonium cation and a tetraalkylammonium cation.

In a case of the salt structure, the type of salt may be one or a mixture of two or more thereof, salt-type and liberated acid-structured groups may be mixed in a compound, or a salt-structured compound and a liberated acid-structured compound may be mixed.

In the present invention, in a case of a plurality of substituents, linking groups, constitutional units, and the like (hereinafter, referred to as a substituent and the like) represented by a specific reference or formula, or in a case of simultaneously defining a plurality of the substituent and the like, unless otherwise specified, the substituent and the like may be the same or different from each other (regardless of the presence or absence of an expression “each independently”, the substituent and the like may be the same or different from each other). The same applies to the definition of the number of substituents and the like. In a case where a plurality of substituents and the like are near (particularly, adjacent to each other), unless otherwise specified, the substituents and the like may be linked to each other to form a ring. In addition, unless otherwise specified, a ring, for example, an alicyclic ring, an aromatic ring, or a heterocyclic ring may be further condensed to form a fused ring.

In the present invention, unless otherwise specified, with regard to a double bond, in a case where E-form and Z-form are present in the molecule, the double bond may be any one of these forms, or may be a mixture thereof.

In addition, in the present invention, unless otherwise specified, in a case where a compound has one or two or more asymmetric carbons, for such stereochemistry of asymmetric carbons, either an (R)-form or an (S)-form can be independently taken. As a result, the compound may be a mixture of optical isomers or stereoisomers such as diastereoisomers, or may be racemic.

In addition, in the present invention, the expression of the compound means that a compound having a partially changed structure is included within a range which does not impair the effects of the present invention. Further, a compound which is not specifically described as substituted or unsubstituted may have an optional substituent within a range which does not impair the effects of the present invention.

In the present invention, with regard to a substituent (the same applies to a linking group and a ring) in which whether it is substituted or unsubstituted is not specified, within a range not impairing the desired effect, it means that the group may have an optional substituent, and the number of substituents which may be included is not particularly limited. For example, “alkyl group” means to include both an unsubstituted alkyl group and a substituted alkyl group. Similarly, “aryl group” means to include both an unsubstituted aryl group and a substituted aryl group.

In the present invention, in a case where the number of carbon atoms in a certain group is specified, the number of carbon atoms means the number of carbon atoms in the entire group, unless otherwise specified in the present invention or the present specification. That is, in a case of a form in which the group has a substituent, it means the total number of carbon atoms including the substituent.

In the present invention, a numerical range represented by using “to” means a range including numerical values described before and after “to” as a lower limit value and an upper limit value.

In the present invention, each component may be used alone or in combination of two or more thereof.

In a description of the content of each component in the curable resin composition according to the aspect of the present invention, in a case where the curable resin composition includes a solvent, the content of each component is based on the component composition obtained by removing the solvent from the curable resin composition. For example, in a case where a curable resin composition is composed of 20 parts by mass of a solvent, 40 parts by mass of a component A, and 40 parts by mass of a component B, for a total of 100 parts by mass, since the content of the component A in the composition is based on 80 parts by mass excluding the solvent, the content thereof is 50% by mass.

In the present invention, “(meth)acrylate” represents either one or both of acrylate and methacrylate, and “(meth)acryloyl” represents either one or both of acryloyl and methacryloyl. The monomer in the present invention is distinguished from an oligomer and a polymer, and refers to a compound having a weight-average molecular weight of 1,000 or less.

In the present invention, the term aliphatic hydrocarbon group means a group obtained by removing one optional hydrogen atom from a linear or branched alkane, a linear or branched alkene, or a linear or branched alkyne. In the present invention, the aliphatic hydrocarbon group is preferably an alkyl group obtained by removing one optional hydrogen atom from a linear or branched alkane.

Examples of the alkyl group include a methyl group, an ethyl group, a propyl group, an isopropyl group, a butyl group, an isobutyl group, a sec-butyl group, a tert-butyl group, a pentyl group, a 1-methylbutyl group, a 3-methylbutyl group, a hexyl group, a 1-methylpentyl group, a 4-methylpentyl group, a heptyl group, a 1-methylhexyl group, a 5-methylhexyl group, a 2-ethylhexyl group, an octyl group, a 1-methylheptyl group, a nonyl group, a 1-methyloctyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group.

In addition, in the present invention, the aliphatic hydrocarbon group (unsubstituted) is preferably an alkyl group having 1 to 20 carbon atoms, and more preferably an alkyl group having 1 to 12 carbon atoms.

In the present invention, the term alkyl group means a linear or branched alkyl group. Examples of the alkyl group include the above-described examples. The same applies to an alkyl group in a group (an alkoxy group, an alkoxycarbonyl group, an acyl group, and the like) including the alkyl group.

In addition, in the present invention, examples of a linear alkylene group include a group obtained by removing one hydrogen atom bonded to a terminal carbon atom from a linear alkyl group among the above-described alkyl groups.

In the present invention, the term alicyclic hydrocarbon ring means a saturated hydrocarbon ring (cycloalkane). Examples of the alicyclic hydrocarbon ring include cyclopropane, cyclobutane, cyclopentane, cyclohexane, cycloheptane, cyclooctane, cyclononane, and cyclodecane.

In the present invention, the term unsaturated hydrocarbon ring means a hydrocarbon ring having a carbon-carbon unsaturated double bond, which is not an aromatic ring. Examples of the unsaturated hydrocarbon ring include indene, indane, and fluorene.

In the present invention, the term alicyclic hydrocarbon group means a cycloalkyl group obtained by removing one optional hydrogen atom from a cycloalkane. Examples of the alicyclic hydrocarbon group include a cyclopropyl group, a cyclobutyl group, a cyclopentyl group, a cyclohexyl group, a cycloheptyl group, a cyclooctyl group, a cyclononyl group, and a cyclodecyl group, and a cycloalkyl group having 3 to 12 carbon atoms is preferable.

In the present invention, a cycloalkylene group refers to a divalent group obtained by removing two optional hydrogen atoms from a cycloalkane. Examples of the cycloalkylene group include a cyclohexylene group.

In the present invention, the term aromatic ring means either one or both of an aromatic hydrocarbon ring and an aromatic heterocyclic ring.

In the present invention, the term aromatic hydrocarbon ring means an aromatic ring in which a ring is formed only by carbon atoms. The aromatic hydrocarbon ring may be a monocyclic ring or a fused ring. Examples of the aromatic hydrocarbon ring include benzene, biphenyl, biphenylene, naphthalene, anthracene, and phenanthrene. In the present invention, in a case where the aromatic hydrocarbon ring is bonded to another ring, it is sufficient that the aromatic hydrocarbon ring may be substituted on another ring as a monovalent or divalent aromatic hydrocarbon group.

In addition, in the present invention, the unsubstituted aromatic hydrocarbon ring is preferably an aromatic hydrocarbon ring having 6 to 14 carbon atoms.

In the present invention, the term monovalent aromatic hydrocarbon group (also referred to as an aryl group) means a monovalent group obtained by removing one optional hydrogen atom from the aromatic hydrocarbon ring. Examples of the monovalent aromatic hydrocarbon group include a phenyl group, a biphenyl group, a 1-naphthyl groups, a 2-naphthyl groups, a 1-anthracenyl group, a 2-anthracenyl group, a 3-anthracenyl group, a 4-anthracenyl group, a 9-anthracenyl group, a 1-phenanthryl group, a 2-phenanthryl group, a 3-phenanthryl group, a 4-phenanthryl group, and a 9-phenanthryl group. Among these, a phenyl group, a 1-naphthyl group, or a 2-naphthyl group is preferable.

In the present invention, the term divalent aromatic hydrocarbon group means a divalent group obtained by removing two optional hydrogen atoms from the aromatic hydrocarbon ring. Examples of the divalent aromatic hydrocarbon group include a divalent group obtained by removing one optional hydrogen atom from the above-described monovalent aromatic hydrocarbon group. Among these, a phenylene group is preferable, and a 1,4-phenylene group is more preferable.

In the present invention, an aromatic heterocyclic ring means an aromatic ring in which a ring is formed by a carbon atom and a heteroatom. Examples of the heteroatom include an oxygen atom, a nitrogen atom, and a sulfur atom. The aromatic heterocyclic ring may be a monocyclic ring or a fused ring, and the number of atoms constituting the ring is preferably 5 to 20 and more preferably 5 to 14. The number of heteroatoms in the atoms constituting the ring is not particularly limited, but is preferably 1 to 3 and more preferably 1 or 2. Examples of the aromatic heterocyclic ring include a furan ring, a thiophene ring, a pyrrole ring, imidazole, isothiazole, isoxazole, pyridine, pyrazine, quinoline, benzofuran, benzothiazole, benzoxazole, and examples of nitrogen-containing fused aromatic ring described later. In the present invention, in a case where the aromatic heterocyclic ring is bonded to another ring, it is sufficient that the aromatic heterocyclic ring may be substituted on another ring as a monovalent or divalent aromatic heterocyclic group.

In the present invention, the term monovalent aromatic heterocyclic group (also referred to as a heteroaryl group) means a monovalent group obtained by removing one optional hydrogen atom from the aromatic heterocyclic ring. Examples of the monovalent aromatic heterocyclic group include a furyl group, a thienyl group (preferably, a 2-thienyl group), a pyrrolyl group, an imidazolyl group, an isothiazolyl group, an isooxazolyl group, a pyridyl group, a pyrazinyl group, a quinolyl group, a benzofuranyl group (preferably, a 2-benzofuranyl group), a benzothiazolyl group (preferably, a 2-benzothiazolyl group), and a benzoxazolyl group (preferably, a 2-benzoxazolyl group). Among these, a furyl group, a thienyl group, a benzofuranyl group, a benzothiazolyl group, or a benzoxazolyl group is preferable, and a 2-furyl group or a 2-thienyl group is more preferable.

In the present invention, the term divalent aromatic heterocyclic group means a divalent group obtained by removing two optional hydrogen atoms from the aromatic heterocyclic ring. Examples of the divalent aromatic heterocyclic group include a divalent group obtained by removing one optional hydrogen atom from the above-described monovalent aromatic heterocyclic group.

In the present invention, examples of a halogen atom include a fluorine atom, a chlorine atom, a bromine atom, and an iodine atom.

The curable resin composition according to the aspect of the present invention is a curable resin composition containing ITO particles and a near-ultraviolet light-absorbing organic compound, and is excellent in medium- to long-term dispersion stability.

### BRIEF DESCRIPTION OF THE DRAWINGS

The FIGURE is a diagram showing absorption spectra of a compound I-37 which satisfies relational expressions I to III described later and a compound C-1 which does not satisfy the relational expression III described later.

### DESCRIPTION OF THE PREFERRED EMBODIMENTS

### Curable Resin Composition

A curable resin composition according to the embodiment of the present invention includes at least a near-ultraviolet light-absorbing organic compound having specific light-absorbing properties, indium tin oxide (ITO) particles, and a polymer having a specific structure.

The curable resin composition according to the embodiment of the present invention means a composition which has curing properties and with which a cured product (resin) can be obtained by curing.

The curable resin composition according to the embodiment of the present invention may include other components in addition to these components. Hereinafter, each component will be described.

### Near-Ultraviolet Light-Absorbing Organic Compound

The curable resin composition according to the embodiment of the present invention includes a near-ultraviolet light-absorbing organic compound which exhibits light absorption in a near-ultraviolet wavelength region. The above-described light absorption of the near-ultraviolet light-absorbing organic compound does not extend to a visible light region, and the near-ultraviolet light-absorbing organic compound exhibits substantially no light absorption at a wavelength of 430 to 800 nm. By adding such a near-ultraviolet light-absorbing organic compound to a curable resin composition including indium tin oxide particles, even in a case where the amount of indium tin oxide particles added is small, it is possible to obtain a chromatic aberration reducing effect in a wide wavelength range in a case where the curable resin composition is used as a material having a low refractive index and a low Abbe number in a multilayer diffractive optical element. Since the amount of indium tin oxide particles added can be reduced, it is possible to suppress a decrease in transmittance in the near-infrared wavelength region.

Specifically, in the near-ultraviolet light-absorbing organic compound, in a case where an absorbance is measured from a wavelength of 800 nm, a wavelength at which the maximal value is first exhibited is present at 300 to 400 nm. That is, the absorption spectrum in a wavelength region of 300 to 800 nm has an absorbance peak having a maximal value only in a range of 300 to 400 nm. The maximal value in the range of 300 to 400 nm may be one or two or more. In a case where the absorbance is measured from a wavelength of 800 nm toward the short wavelength side, the wavelength at which the maximal value is first exhibited is present preferably at 340 to 390 nm and more preferably at 350 to 380 nm. In addition, the maximal value exhibiting the highest absorbance among maximal values in the range of 300 to 400 nm is preferably 340 to 385 nm and more preferably 350 to 380 nm. Here, it is sufficient that the absorption spectrum is measured with a solution of the near-ultraviolet light-absorbing organic compound, and it is assumed that the absorption spectrum is obtained by placing a solvent-only cell in a sample optical path and a control optical path to adjust the absorbance to zero, and then replacing the sample optical path-side cell with a solution of the near-ultraviolet light-absorbing organic compound for measurement. Details can be measured based on the methods described in Examples later.

In addition, in the above-described near-ultraviolet light-absorbing organic compound, in a case where an absorbance at a wavelength of λ nm is defined as A_{λ}, relationships of Expression I to III are satisfied. Specifically, among maximal values in the range of 300 to 400 nm, an absorbance A_{λmax} (also referred to as “maximum absorbance at 300 to 400 nm” in the present invention) at a wavelength λmax with the highest absorbance, an absorbance A₄₁₀ at a wavelength 410 nm of the above-described absorption spectrum, and an absorbance A₄₃₀ at a wavelength 430 nm of the above-described absorption spectrum satisfy the following relational expressions.

$$\left({\text{A}}_{λ max} \text{-} {\text{A}}_{410}\right) / {\text{A}}_{λ max} \geq 0.97$$

$$1.00 \geq \left({\text{A}}_{λ max} \text{-} {\text{A}}_{410}\right) / \left({\text{A}}_{λ max} \text{-} {\text{A}}_{430}\right) \geq 0.97$$

$$\left({\text{A}}_{λ max} \text{-} {\text{A}}_{410}\right) / \left(410 \text{-} λ \text{max}\right) \geq 0.005$$

It is preferable that Expressions I and II satisfy the following expressions in order.

$$\left({\text{A}}_{λ max} \text{-} {\text{A}}_{410}\right) / {\text{A}}_{λ max} \geq 0.98$$

$$1.00 \geq \left({\text{A}}_{λ max} \text{-} {\text{A}}_{410}\right) / \left({\text{A}}_{λ max} \text{-} {\text{A}}_{430}\right) \geq 0.98$$

That is, both A₄₁₀ and A₄₃₀ are values that are considerably smaller (values close to 0) with respect to A_{λmax}.

As shown in the Figure, for example, with respect to an exemplary compound I-37 of a compound represented by General Formula (1) described later, the following compound C-1, which has a fluorene structure as a near-ultraviolet light-absorbing moiety, does not satisfy the above-described relational expression III. With such a compound C-1, a low Abbe number cannot be achieved.

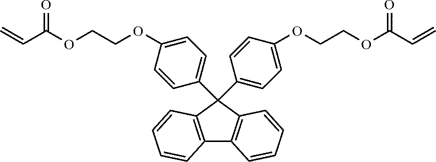

The measurement conditions of the absorption spectra are not particularly limited. As an example, using a 20 mg/L solution of the near-ultraviolet light-absorbing organic compound, the absorption spectrum can be measured using UV-2550 (product name) manufactured by Shimadzu Corporation with an optical path length of 10 mm. However, Expression III is a relational expression satisfying this measurement condition.

The solvent used for measuring the absorption spectrum is not particularly limited as long as the solvent can dissolve the near-ultraviolet light-absorbing organic compound, and for example, tetrahydrofuran can be used.

The near-ultraviolet light-absorbing organic compound included in the curable resin composition according to the embodiment of the present invention is preferably a polymerizable compound. That is, the near-ultraviolet light-absorbing organic compound is preferably a compound having a polymerizable group.

### Polymerizable Group

The polymerizable group may be a group including any of a vinylidene structure, an oxirane structure, or an oxetane structure. From the viewpoint of convenience and the like in synthesizing the near-ultraviolet light-absorbing organic compound, the polymerizable group is preferably a group in which the linking part is an oxygen atom and which includes any of a vinylidene structure, an oxirane structure, or an oxetane structure, and examples thereof include polymerizable groups represented by any of Formulae (Pol-1) to (Pol-6).

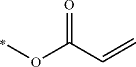

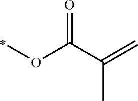

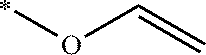

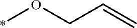

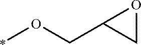

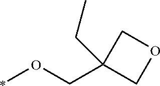

*represents bonding position.

Among these, a (meth)acryloyloxy group represented by Formula (Pol-1) or Formula (Pol-2) is preferable.

The near-ultraviolet light-absorbing organic compound may have 1 or more polymerizable groups, and preferably has 1 to 4 polymerizable groups and more preferably has 1 or 2 polymerizable groups.

The near-ultraviolet light-absorbing organic compound included in the curable resin composition according to the embodiment of the present invention is preferably a compound including an aromatic ring as a partial structure, more preferably at least one of the following compounds 1 to 3, and from the viewpoint of realizing a lower Abbe number, still more preferably the following compound 1 or 2.

### Compound 1

The compound 1 preferred as the above-described near-ultraviolet light-absorbing organic compound is a compound represented by General Formula (1). The compound represented by General Formula (1) includes a benzene ring with a fused ring of benzene and a heterocyclic ring, such as benzodithiol and benzothiazole, a hydrazone, or the like as a substituent in its structure. The present inventors have found that the compound represented by General Formula (1) has the above-described spectral characteristics, and a cured product obtained from the curable resin composition containing the compound represented by General Formula (1) has a low Abbe number (vd). Further, the present inventors have also found that the cured product obtained from the curable resin composition containing the compound represented by General Formula (1) has a high heat shock resistance, that is, ability to relax stress during thermal changes in the cured product.

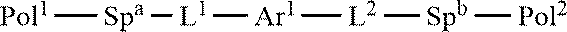

In the formula, Ar¹ represents an aromatic ring group represented by any of General Formula (2-1), ..., or (2-4). L¹ and L² represent a single bond, —O—, —S—, —C(═O)—, —OC(═O)—, —C(═O)O—, —OC(═O)O—, —NR¹⁰¹C(═O)—, —C(═O)NR¹⁰²—, —OC(═O)NR¹⁰³—, —NR¹⁰⁴C(═O)O—, —SC(═O)—, or —C(═O)S—. R¹⁰¹ to R¹⁰⁴ represent -Sp^{c}-Pol³. Sp^{a} represents a linking group having a shortest atom number of 2 or more and linking Pol¹ and L¹, Sp^{b} represents a linking group having the shortest atom number of 2 or more and linking Pol² and L², and Sp^{c} represents a single bond or a divalent linking group. Pol¹ to Pol³ represent a hydrogen atom or a polymerizable group, in which at least one of Pol¹ or Pol² represents a polymerizable group. However, a linking portion of Sp^{a} to L¹ and a linking portion of Sp^{b} to L² are both —CH₂—. In addition, a linking portion of Sp^{a} to Pol¹, a linking portion of Sp^{b} to Pol², and a linking portion of Sp^{c} to Pol³ are all a carbon atom.

Hereinafter, Ar¹, Sp^{a} and Sp^{b}, Pol¹ and Pol², and L¹ and L² will be described in detail.

### Ar¹

Ar¹ is an aromatic ring group represented by any of General Formula (2-1), ..., or (2-4).

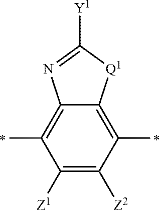

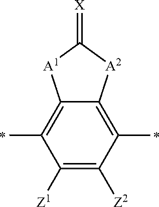

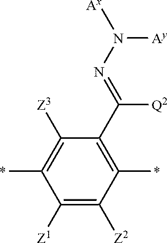

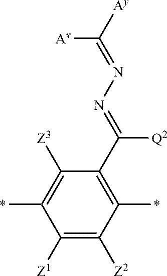

In the formula, Q¹ represents —S—, —O—, or >NR¹¹, and R¹¹ represents a hydrogen atom or an alkyl group having 1 to 6 carbon atoms.
Y¹ represents an alkyl group having 1 to 6 carbon atoms, an aromatic hydrocarbon group having 6 to 12 carbon atoms, or an aromatic heterocyclic group having 3 to 12 carbon atoms.
Z¹, Z², and Z³ represent a hydrogen atom, an aliphatic hydrocarbon group having 1 to 20 carbon atoms, an alkoxy group having 1 to 20 carbon atoms, an alicyclic hydrocarbon group having 3 to 20 carbon atoms, an aromatic hydrocarbon group having 6 to 20 carbon atoms, a halogen atom, a cyano group, a nitro group, —NR¹²R¹³, or —SR¹². Z¹ and Z² may be bonded to each other to form an aromatic hydrocarbon ring or an aromatic heterocyclic ring. R¹² and R¹³ represent a hydrogen atom or an alkyl group having 1 to 6 carbon atoms.
A¹ and A² represent —O—, >NR²¹, —S—, or >C(═O). R²¹ represents a hydrogen atom or a substituent, in which a hydrogen atom or an alkyl group having 1 to 6 carbon atoms is preferable.
X represents ═O (oxygen atom), ═S (sulfur atom), a carbon atom to which a hydrogen atom or a substituent is bonded, or a nitrogen atom to which a hydrogen atom or a substituent is bonded.
A^{x} represents an organic group having 1 to 30 carbon atoms, which has at least one aromatic ring selected from an aromatic hydrocarbon ring or an aromatic heterocyclic ring. A^{y} represents a hydrogen atom, an alkyl group having 1 to 6, or an organic group having 1 to 30 carbon atoms, which has at least one aromatic ring selected from an aromatic hydrocarbon ring or an aromatic heterocyclic ring. A^{x} and A^{y} may be bonded to each other to form a ring.
Q² represents a hydrogen atom or an alkyl group having 1 to 6 carbon atoms.
* represents a bonding position with L¹ or L².

With regard to the definition and preferred range of each substituent in General Formulae (2-1) to (2-4), unless otherwise noted, the descriptions regarding Y¹, Q¹, and Q² in the compound (A) described in JP2012-21068A can be adopted as they are to Y¹, Z¹, and Z²; and the descriptions regarding A₁, A₂, and X in the compound represented by General Formula (I) described in JP2008-107767A can be adopted as they are to A¹, A^{Z}, and X in General Formula (2-2). In addition, the descriptions regarding A^{x}, A^{y}, and Q¹ in the compound represented by General Formula (I) described in WO2013/018526A can be adopted as they are to A^{x}, A^{y}, and Q² in General Formula (2-3); and the descriptions regarding A^{a}, A^{b}, and Q¹¹ in the compound represented by General Formula (II) described in WO2013/018526A can be adopted as they are to A^{x}, A^{y}, and Q² in General Formula (2-4). The description regarding Q¹ in the compound (A) described in JP2012-21068A can be adopted as they are to Z³.

X in General Formula (2-2) is preferably a carbon atom to which two substituents are bonded, and both A¹ and A² are preferably —S—. In General Formula (2-3), as the ring in a case where A^{x} and A^{y} are bonded to each other to form a ring, an alicyclic hydrocarbon ring, an aromatic hydrocarbon ring, or an aromatic heterocyclic ring is preferable, and an aromatic heterocyclic ring is more preferable. In General Formula (2-4), as the ring in a case where A^{x} and A^{y} are bonded to each other to form a ring, an unsaturated hydrocarbon ring is preferable.

Ar¹ in General Formula (1) is preferably the aromatic ring group represented by General Formula (2-2).

As the aromatic ring group represented by General Formula (2-2), an aromatic ring group represented by General Formula (2-21) is preferable.

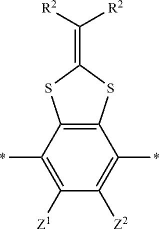

In the formula, R^{Z} represents a substituent, and Z¹ and Z² have the same meaning as Z¹ and Z² in General Formula (2-2), respectively.

Examples of the substituent represented by R^{Z} include substituents which may be included in a linear alkylene group in Sp^{a} and Sp^{b}, which will be described later, and preferred examples thereof include an alkyl group, an alkoxy group, an alkoxycarbonyl group, a halogen atom, and a cyano group. Two R^{Z}’s may be the same or different from each other.

In addition, two R^{Z}’s may be bonded to each other to form a ring, and in this case, the ring to be formed is preferably a 5-membered ring or a 6-membered ring, and more preferably includes a nitrogen atom or an oxygen atom as an atom constituting the ring. The ring formed by bonding two R^{Z}’s to each other is more preferably a ring represented by any of the following structures.

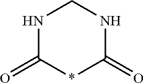 [SMILES: O=C1*C(=O)NCN1]

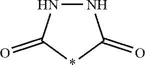 [SMILES: O=c1*c(=O)[nH][nH]1]

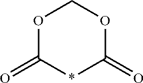 [SMILES: O=C1*C(=O)OCO1]

In the above formulae, * represents a position of a carbon atom where the two R^{Z}’s are bonded in General Formula (2-21), respectively. As the substituent in this case, an alkyl group having 1 to 6 carbon atoms is preferable, and a linear alkyl group having 1 to 4 carbon atoms is more preferable.

As the aromatic ring group represented by General Formula (2-21), an aromatic ring group in which at least one of R^{Z}’s is a cyano group or an aromatic ring group in which two R^{Z}’s are bonded to each other to form a ring is preferable, and from the viewpoint of further improving light resistance of the cured product, an aromatic ring group represented by General Formula (2-21a), in which two R^{Z}’s are cyano groups, is more preferable.

In a case where the Ar¹ is an aromatic ring group represented by General Formula (2-21a), the adhesiveness can be further improved.

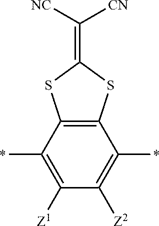

In the formula, Z¹ and Z² have the same meaning as Z¹ and Z² in General Formula (2-2), respectively.

### Sp^{a} and Sp^{b}

Sp^{a} represents a linking group having a shortest atom number of 2 or more and linking Pol¹ and L¹, and Sp^{b} represents a linking group having the shortest atom number of 2 or more and linking Pol² and L². However, a linking portion of Sp^{a} to L¹ and a linking portion of Sp^{b} to L² are both —CH₂—, and a linking portion of Sp^{a} to Pol¹ and a linking portion of Sp^{b} to Pol² are both a carbon atom. The regulation of these linking portions also applies to the following descriptions relating to Sp^{a} and Sp^{b}.

As an example of the above-described “linking group having the shortest atom number of 2 or more”, in —L²—Sp^{b}—Pol² shown below, the shortest number of atoms linking —O— as L² and a methacryloyloxy group as Pol² is 10.

The above-described shortest atom number is preferably 2 to 30, more preferably 2 to 20, and still more preferably 2 to 16.

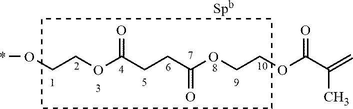

As the above-described linking group represented by Sp^{a} or Sp^{b}, a linear alkylene group having 2 to 30 carbon atoms or a group in which, in a linear alkylene group having 2 to 30 carbon atoms, one or two or more —CH₂—’s excluding a linking portion to L¹ or L² are substituted by a group selected from —O—, —S—, >C(═O), and >NR¹¹¹ is preferable, and a linear alkylene group having 2 to 30 carbon atoms or a group in which, in a linear alkylene group having 2 to 30 carbon atoms, one or two or more —CH₂—’s excluding a linking portion to L¹ or L² are substituted by a group selected from —O— and >C(═O) is more preferable.

The R¹¹¹ represents a hydrogen atom or an alkyl group having 1 to 3 carbon atoms.

The carbon atoms in the above-described “linear alkylene group having 2 to 30 carbon atoms” mean the carbon number in a state without a substituent. Therefore, as the carbon number in the linear alkylene group having 2 to 30 carbon atoms, the preferred carbon number described in the above-described shortest atom number can be adopted. In this regard, in a case where the “linear alkylene group having 2 to 30 carbon atoms” has a substituent, an alkyl group can also be taken as the substituent. In this case, the alkylene group is a branched alkylene group as a whole, but a linear moiety consisting of the “shortest atom number” of the above-described “shortest atom number of 2 or more” in Sp^{a} and Sp^{b} corresponds to the “linear alkylene group having 2 to 30 carbon atoms”.

Examples of the substituent which may be included in the linear alkylene group of Sp^{a} and Sp^{b} described above include an alkyl group, a cycloalkyl group, an alkoxy group, an acyl group, an acyloxy group, an alkoxycarbonyl group, an amide group, an amino group, a halogen atom, a nitro group, and a cyano group, and an alkyl group is preferable, an alkyl group having 1 to 3 carbon atoms is more preferable, and a methyl group is still more preferable.

The number of substituents is not particularly limited, and for example, may be 1 to 4.

The above-described substitution of one or two or more —CH₂—’s, excluding the linking portion to L¹ or L² by the group selected from —O—, —S—, >C(═O), and >NR¹¹¹ is not particularly limited in the number, type, and the like of the substitution as long as the substitution can function as the linking group.

Specific examples of the above-described substitution are shown below.

“Substitution of —CH₂—”:

Examples thereof include a substitution of —CH₂— by —O—, —S—, >C(═O), or >NR¹¹¹, and a substitution by —O— or >C(═O) is preferable, and a substitution by —O— is more preferable.

“Substitution of —CH₂CH₂—”:

Examples thereof include a substitution of —CH₂CH₂— by —C(═O)O—, —NR¹¹¹C(═O)—, or —SC(═O)—, and a substitution by —C(═O)O— or —NR¹¹¹C(═O)— is preferable, and a substitution by —C(═O)O— is more preferable.

“Substitution of —CH₂CH₂CH₂—”:

Examples thereof include a substitution of —CH₂CH₂CH₂— by —OC(═O)O—, —NR¹¹¹C(═O)O—, and a substitution by —OC(═O)O— is preferable.

The above-described substitution by —C(═O)O—, —NR¹¹¹C(═O)—, —NR¹¹¹C(═O)O—, or —SC(═O)— may be substituted in a form such that either the left or right bonding site is located on the L¹ side or the L² side.

As the above-described linking group represented by Sp^{a} or Sp^{b}, from the viewpoint of further improving light resistance of the cured product, a linear alkylene group having 2 to 30 carbon atoms or a group in which, in a linear alkylene group having 2 to 30 carbon atoms, one or two or more —CH₂CH₂—’s excluding a linking portion to L¹ or L² are substituted by a group selected from —C(═O)O— and —OC(═O)— is still more preferable.

In a case where the above-described linking group represented by Sp^{a} or Sp^{b} is the still more preferred group, the adhesiveness can be further improved.

Sp^{a} and Sp^{b} may be the same or different from each other, but it is preferable that Sp^{a} and Sp^{b} are the same.

### Pol¹ and Pol²

Pol¹ and Pol² represent a hydrogen atom or a polymerizable group, and any one of Pol¹ or Pol² is a polymerizable group.

The polymerizable group which can be adopted as Pol¹ or Pol² has the same meaning as the above-described polymerizable group.

It is preferable that any one of Pol¹ or Pol² is a (meth)acryloyloxy group, and it is more preferable that the both are (meth)acryloyloxy groups.

Pol¹ and Pol² may be the same or different from each other, but it is preferable that Pol¹ and Pol² are the same.

Examples of a specific structure of Pol¹—Sp^{a}—L¹— or Pol²—Sp^{b}—L²— include the following structures.

Pol¹—Sp^{a}—L¹— and Pol²—Sp^{b}—L²— may be the same or different from each other, but it is preferable that Pol¹—Sp^{a}—L¹— and Pol²—Sp^{b}—L²— are the same.

In the following structures, R is a hydrogen atom or a methyl group. In addition, * represents a bonding position with Ar¹.

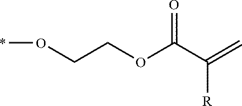

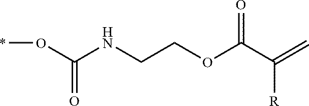

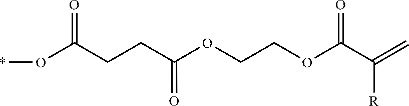

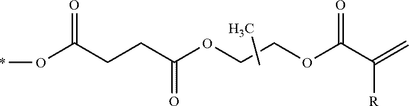

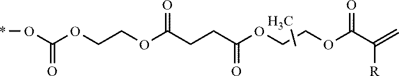

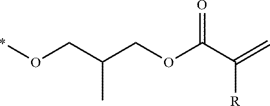

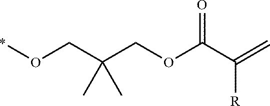

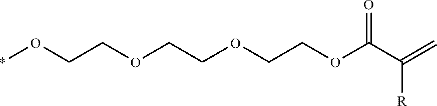

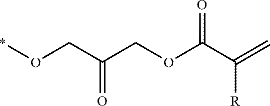

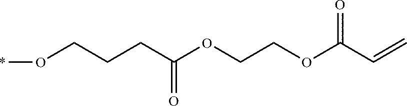 [SMILES: *OCCCC(=O)OCCOC(=O)C=C]

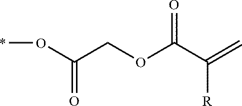

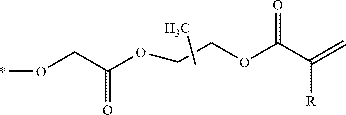

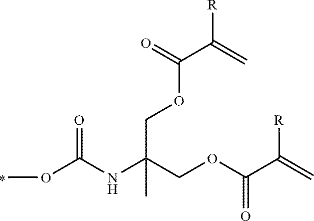

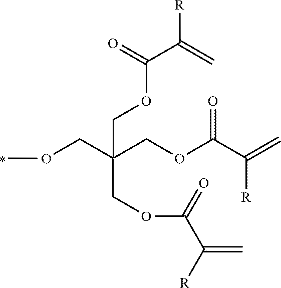

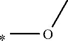

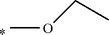

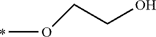

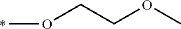

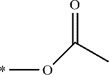

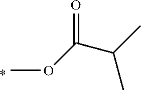

In the present invention, the structure represented by the following notation indicates an isopropylene structure. This isopropylene structure may be any of two structural isomers in which a methyl group is bonded to one of carbons constituting an ethylene group, and these structural isomers may be mixed.

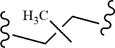

or

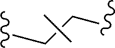

As described above, in the compound represented by General Formula (1), in a case where a linear alkylene group has a structure in which a substituent is substituted, structural isomers having different substitution positions of the substituent may exist. The compound represented by General Formula (1) may be a mixture of such structural isomers.

### L¹ and L²

L¹ and L² represent a single bond, —O—, —S—, —C(═O)—, —OC(═O)—, —C(═O)O—, —OC(═O)O—, —NR¹⁰¹C(═O)—, —C(═O)NR¹⁰²—, —OC(═O)NR¹⁰³—, —NR¹⁰⁴C(═O)O—, —SC(═O)—, or —C(═O)S—. In the above description of the linking group, it is assumed that the left side is bonded to Ar¹ and the right side is bonded to Sp^{a} or Sp^{b}.

R¹⁰¹ to R¹⁰⁴ represent -Sp^{c}-Pol³. Sp^{c} represents a single bond or a divalent linking group, and Pol³ represents a hydrogen atom or a polymerizable group.

Examples of the divalent linking group which can be adopted as Sp^{c} include the following linking groups and linking groups consisting of two or more of the following linking groups: linear alkylene groups; cycloalkylene groups (for example, a trans-1,4-cyclohexylene group); divalent aromatic hydrocarbon groups (for example, a 1,4-phenylene group); divalent aromatic heterocyclic groups; —O—; —S—; —C(═O)—; —OC(═O)—; —C(═O)O—; —OC(═O)O—; —NR²⁰¹C(═O)—; —C(═O)NR²⁰²—; —OC(═O)NR²⁰³—; —NR²⁰⁴C(═O)O—; —SC(═O)—; and —C(═O)S—. Examples of Sp^{c} which is a divalent linking group include a linear alkylene group, a cycloalkylene group, a divalent aromatic hydrocarbon group, and a divalent aromatic heterocyclic group. In addition, examples thereof also include linking groups in which two or more linking groups selected from the linear alkylene group, the cycloalkylene group, the divalent aromatic hydrocarbon group, and the divalent aromatic heterocyclic group are bonded to each other through a linking group selected from a single bond, —O—, —C(═O)—, —OC(═O)—, —C(═O)O—, —OC(═O)O—, —NR²⁰¹C(═O)—, and C(═O)NR²⁰²—.

R²⁰¹ to R²⁰⁴ represent a hydrogen atom or an alkyl group having 1 to 3 carbon atoms.

As the divalent linking group represented by Sp^{c}, a single bond or a linear alkylene group having 1 to 10 carbon atoms is preferable, a linear alkylene group having 1 to 5 carbon atoms is more preferable, a linear alkylene group having 1 to 3 carbon atoms is still more preferable, and an unsubstituted linear alkylene group is particularly preferable.

The polymerizable group which can be adopted as Pol³ has the same meaning as the above-described polymerizable group.

Pol³ is preferably a hydrogen atom.

As —Sp^{c}—Pol³, a hydrogen atom or an alkyl group having 1 to 4 carbon atoms is preferable, and a hydrogen atom or an unsubstituted alkyl group having 1 to 4 carbon atoms is more preferable.

L¹ and L² are preferably —O—, —OC(═O)—, —C(═O)O—, —OC(═O)O—, —NR¹⁰¹C(═O)—, —C(═O)NR¹⁰²—, —OC(═O)NR¹⁰³—, or —NR¹⁰⁴C(═O)O—, more preferably —O—, —OC(═O)—, —OC(═O)O—, or —OC(═O)NR¹⁰³—, still more preferably —O— or —OC(═O)—, and particularly preferably —O—.

R¹⁰¹ to R¹⁰⁴ are preferably a hydrogen atom or an alkyl group having 1 to 4 carbon atoms.

L¹ and L² may be the same or different from each other, but it is preferable that L¹ and L² are the same.

The compound represented by General Formula (1) preferably has at least two polymerizable groups.

The compound represented by General Formula (1) is preferably a non-liquid crystalline compound.

Hereinafter, specific examples of the compound represented by General Formula (1), which is preferably used in the curable resin composition according to the embodiment of the present invention, will be shown, but the present invention is not limited to the following compounds. In the following structural formulae, Me represents a methyl group, Et represents an ethyl group, nPr represents an n-propyl group, iPr represents an isopropyl group, nBu represents an n-butyl group, and tBu represents a t-butyl group.

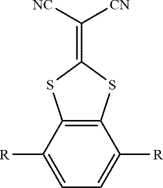

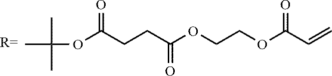

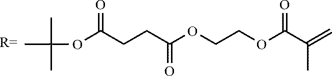

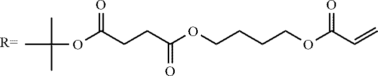

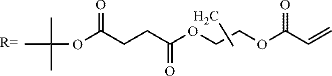

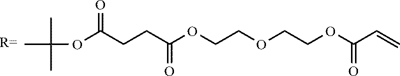

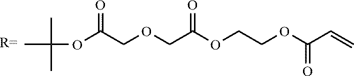

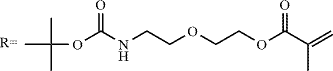

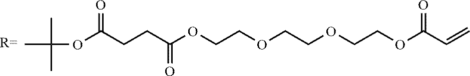

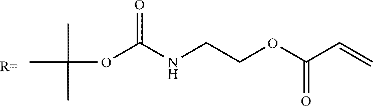

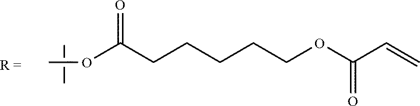

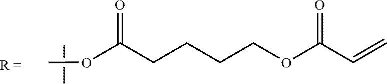

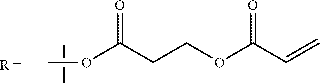

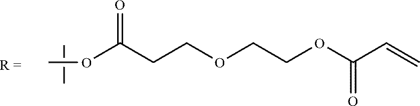

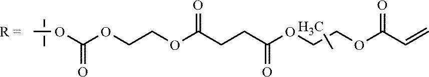

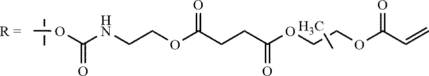

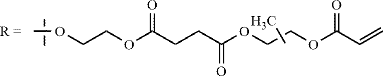

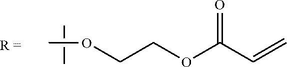

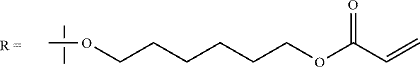

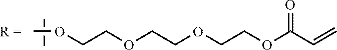

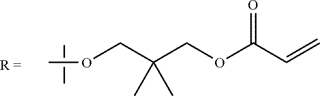

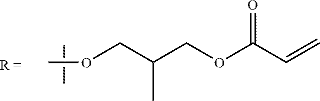

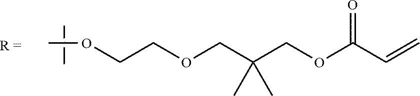

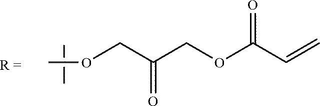

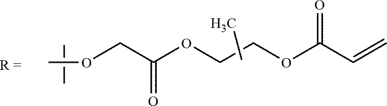

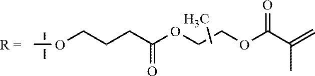

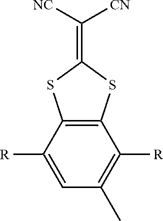

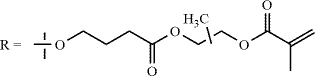

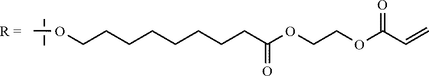

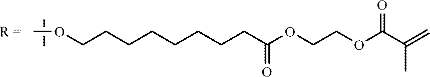

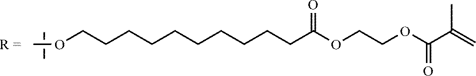

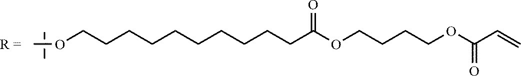

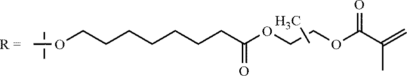

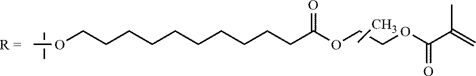

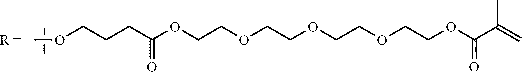

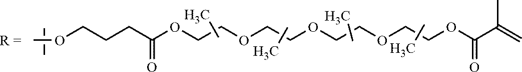

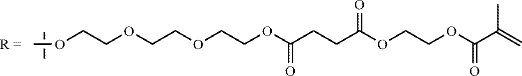

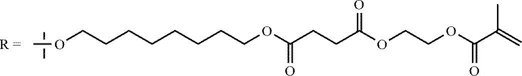

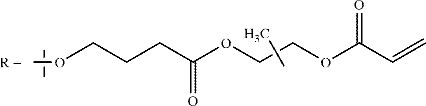

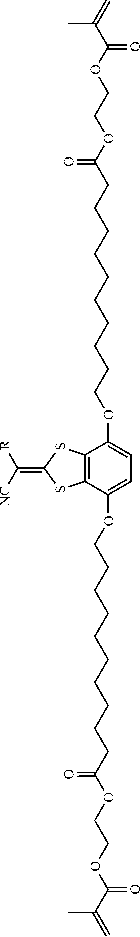

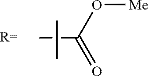

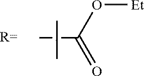

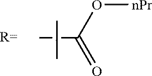

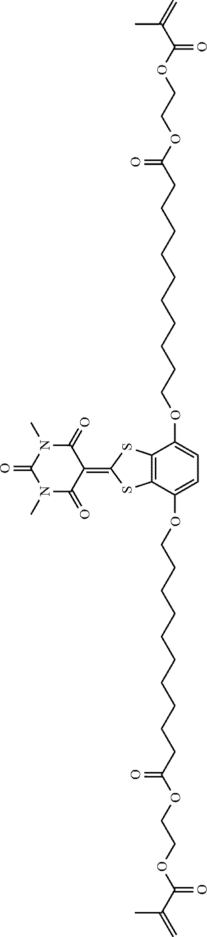 [SMILES: C=C(C)C(=O)OCCOC(=O)CCCCCCCCCCOc1ccc(OCCCCCCCCCCC(=O)OCCOC(=O)C(=C)C)c2c1SC(=C1C(=O)N(C)C(=O)N(C)C1=O)S2]

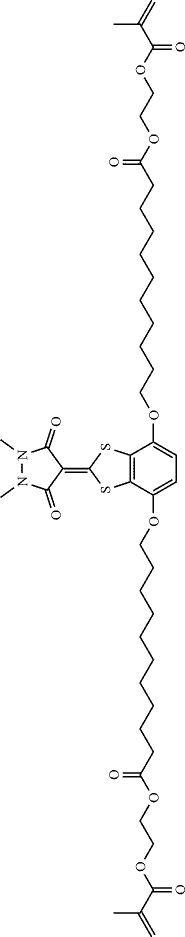 [SMILES: C=C(C)C(=O)OCCOC(=O)CCCCCCCCCCOc1ccc(OCCCCCCCCCCC(=O)OCCOC(=O)C(=C)C)c2c1SC(=C1C(=O)N(C)N(C)C1=O)S2]

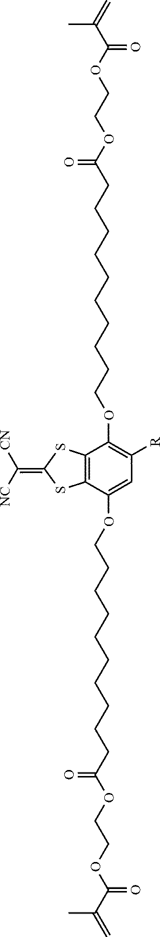

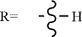

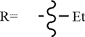

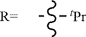

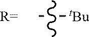

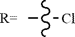

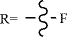

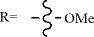

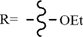

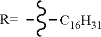

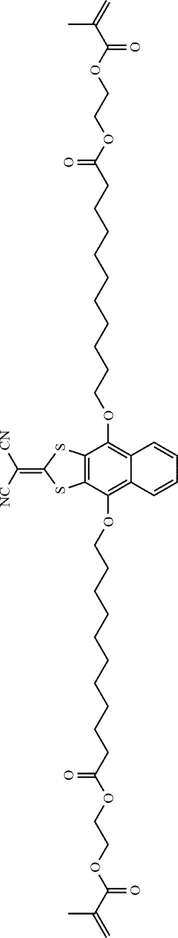 [SMILES: C=C(C)C(=O)OCCOC(=O)CCCCCCCCCCOc1c2c(c(OCCCCCCCCCCC(=O)OCCOC(=O)C(=C)C)c3ccccc13)SC(=C(C#N)C#N)S2]

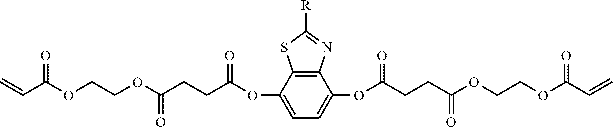

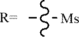

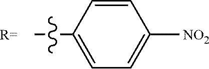

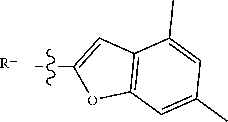

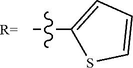

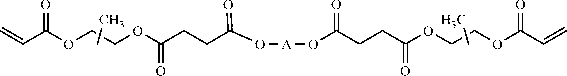

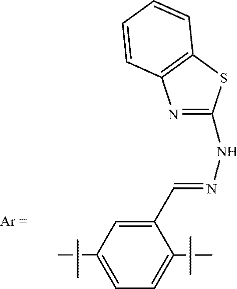

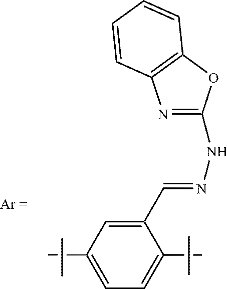

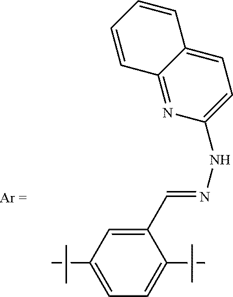

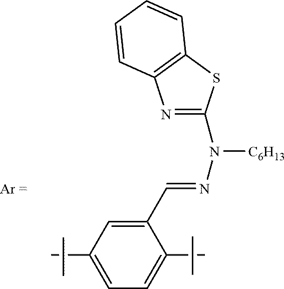

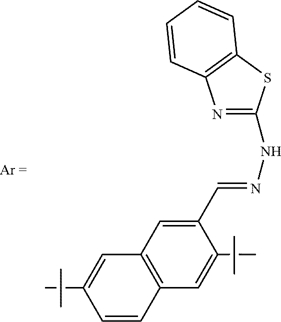

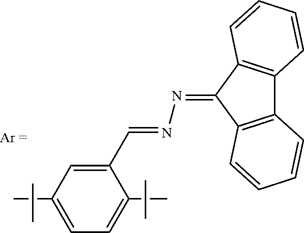

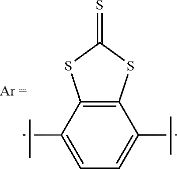

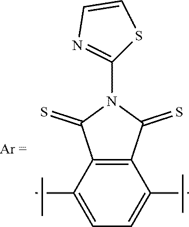

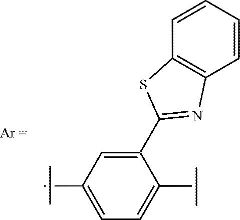

### Compound 2

The compound 2 preferred as the above-described near-ultraviolet light-absorbing organic compound is a compound represented by General Formula (A). The compound represented by General Formula (A) includes a specific nitrogen-containing fused aromatic ring represented by Formula (A1) in its structure. With the compound represented by General Formula (A), it is possible to lower the Abbe number (vd) and increase a partial dispersion ratio (θg, F) of the cured product obtained from the curable resin composition containing the compound.

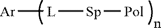

In the formula, Ar represents a group represented by General Formula (A1). L represents a single bond, —O—, —S—, —C(═O)—, —OC(═O)—, —C(═O)O—, —OC(═O)O—, —NR³⁰¹C(═O)—, —C(═O)NR³⁰²—, —OC(═O)NR³⁰³—, —NR³⁰⁴C(═O)O—, —SC(═O)—, or —C(═O)S—. R³⁰¹ to R³⁰⁴ represent —Sp^{d}—Pol⁴. Sp and Sp^{d} represent a single bond or a divalent linking group, and Pol and Pol⁴ represent a hydrogen atom or a polymerizable group.

n is an integer of 1 or 2.

However, the compound represented by General Formula (A) has at least one polymerizable group.

Hereinafter, Ar, L, Sp and Sp^{d}, and Pol and Pol⁴ will be described in detail.

### Ar

The Ar is a group represented by General Formula (A1).

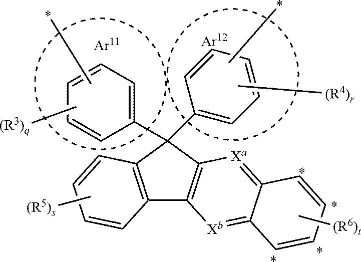

In the formula, Ar¹¹ and Ar¹² represent an aromatic hydrocarbon group including a benzene ring surrounded by a broken line or an aromatic heterocyclic group including a benzene ring surrounded by a broken line as one of rings constituting a fused ring.

X^{a} and X^{b} represent a nitrogen atom or CH, CH at a position of # may be substituted by a nitrogen atom.

R³ to R⁶ represent a substituent, q, r, s, and t are an integer of 0 to 4.

In addition, * represents a bonding position with Pol—Sp—L—.

Ar¹¹ and Ar¹² are preferably an aromatic hydrocarbon group including a benzene ring surrounded by a broken line. In a case where Ar¹¹ and Ar¹² are an aromatic hydrocarbon group including a benzene ring surrounded by a broken line, the aromatic hydrocarbon group is preferably an aromatic hydrocarbon group having 6 to 18 carbon atoms, more preferably an aromatic hydrocarbon group having 6 to 14 carbon atoms, and still more preferably an aromatic hydrocarbon group having 6 to 10 carbon atoms. Among these, Ar¹¹ and Ar¹² are particularly preferably a phenyl group composed of only a benzene ring surrounded by a broken line.

In a case where Ar¹¹ and Ar¹² are an aromatic heterocyclic group including a benzene ring surrounded by a broken line as one of rings constituting the fused ring, the aromatic heterocyclic group is preferably an aromatic heterocyclic group having 9 to 14 ring-constituting atoms, and more preferably an aromatic heterocyclic group having 9 or 10 ring-constituting atoms. In a case where Ar¹¹ and Ar¹² are an aromatic heterocyclic group including a benzene ring surrounded by a broken line as one of fused rings, examples of a heteroatom include a nitrogen atom, an oxygen atom, and a sulfur atom.

The substituent represented by R³ to R⁶ is not particularly limited, and examples thereof include a halogen atom, an alkyl group, an alkenyl group, an acyl group, a hydroxy group, a hydroxyalkyl group, an alkoxy group, an aromatic hydrocarbon group, an aromatic heterocyclic group, an aliphatic ring group, and a cyano group.

The substituent represented by R³ to R⁶ is preferably a halogen atom, an alkyl group, an alkoxy group, an aromatic hydrocarbon group, or a cyano group, more preferably a halogen atom, an alkyl group having 1 to 5 carbon atoms, an alkoxy group having 1 to 5 carbon atoms, a phenyl group, or a cyano group, and still more preferably a halogen atom, a methyl group, a methoxy group, a phenyl group, or a cyano group.

Among these, R³ and R⁴ are preferably a methyl group or a methoxy group, and R⁵ is preferably a halogen atom, a methyl group, or a methoxy group, and more preferably a methyl group. In addition, R⁶ is preferably a halogen atom, a methyl group, or a methoxy group, and more preferably a methyl group.

q and r are preferably 0 or 1, and more preferably 0. s and t are preferably an integer of 0 to 2, and it is more preferable that s is 0 and t is an integer of 0 to 2.

With regard to the substitution position of R⁶ in a case where t is 1 and the substitution position of R⁶ in a case where t is 2, the description of the substitution position of R⁶ in a quinoxaline ring in General Formula (A1-2) below can be applied by replacing it with the substitution position in the fused ring in which the nitrogen atom is represented by R^{a} and R^{b}.

It is preferable that at least one of four CH’s at the positions of X^{a}, X^{b}, and # is substituted by a nitrogen atom.

It is preferable that either one of X^{a} and X^{b} is a nitrogen atom and the other is CH, or both are nitrogen atoms, and it is more preferable that both X^{a} and X^{b} are nitrogen atoms.

In addition, it is preferable that none of CH’s at the position of # is substituted by the nitrogen atom, and in this case, at least one of s or t is preferably an integer of 1 to 4.

The group represented by General Formula (A1) is preferably a group represented by General Formula (A1-2).

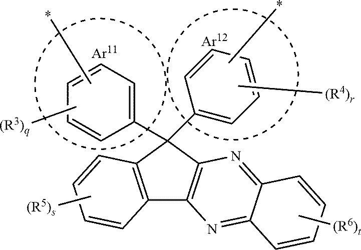

In the above formula, Ar¹¹, Ar¹², R³ to R⁶, q, r, s, t, and * have the same meaning as Ar¹¹, Ar¹², R³ to R⁶, q, r, s, t, and * in Formula (A1) described above. * represents a bonding position with Pol-Sp-L-.

In a case where t is 1, the substitution position of R⁶ is preferably the 6-position or the 7-position of the formed quinoxaline ring, and in a case where t is 2, the substitution position of R⁶ is preferably the 6-position and 7-position of the formed quinoxaline ring.

### L

In General Formula (A), L represents a single bond, —O—, —S—, —C(═O)—, —OC(═O)—, —C(═O)O—, —OC(═O)O—, —NR³⁰¹C(═O)—, —C(═O)NR³⁰²—, —OC(═O)NR³⁰³—, —NR³⁰⁴C(═O)O—, —SC(═O)—, or —C(═O)S—. In the above description of the linking group, it is assumed that the left side is bonded to Ar and the right side is bonded to Sp.

R³⁰¹ to R³⁰⁴ represent —Sp^{d}—Pol⁴. Sp^{d} represents a single bond or a divalent linking group, and Pol⁴ represents a hydrogen atom or a polymerizable group.

R³⁰¹ to R³⁰⁴ are preferably a hydrogen atom or an alkyl group having 1 to 4 carbon atoms.

L’s are each independently preferably —O—, —OC(═O)—, —C(═O)O—, —OC(═O)O—, —NR³⁰¹C(═O)—, —C(═O)NR³⁰²—, —OC(═O)NR³⁰³—, or —NR³⁰⁴C(═O)O—, more preferably —O—, —OC(═O)—, —OC(═O)O—, or —OC(═O)NR³⁰³—, and still more preferably —O— or —OC(═O)—.

In a case where n is 2, a plurality of L’s may be the same or different from each other, but it is preferable that L’s are the same.

### Sp and Sp^{d}

Sp and Sp^{d} represent a single bond or a divalent linking group.

Examples of Sp and Sp^{d} which are a divalent linking group include a linear alkylene group, a cycloalkylene group, a divalent aromatic hydrocarbon group, and a divalent aromatic heterocyclic group. In addition, examples thereof also include linking groups in which two or more linking groups selected from the linear alkylene group, the cycloalkylene group, the divalent aromatic ring group, and the divalent aromatic heterocyclic group are bonded to each other through a linking group selected from a single bond, —O—, —S—, —C(═O)—, —OC(═O)—, —C(═O)O—, —OC(═O)O—, —NR⁴⁰¹C(═O)—, —C(═O)NR⁴⁰²—, —OC(═O)NR⁴⁰³—, —NR⁴⁰⁴C(═O)O—, —SC(═O)—, and —C(═O)S—.

In the above description of the linking group, it is assumed that the left side is bonded to L or N (in a case of Sp^{d}) and the right side is bonded to Pol or Pol⁴ (in a case of Sp^{d}).

R⁴⁰¹ to R⁴⁰⁴ represent a hydrogen atom or an alkyl group having 1 to 3 carbon atoms.

Examples of the substituent which may be included in the substituent in Sp and Sp^{d} include an alkyl group, a cycloalkyl group, an alkoxy group, an acyl group, an acyloxy group, an alkoxycarbonyl group, an amide group, an amino group, a halogen atom, a nitro group, a cyano group, and a substituent formed by combining two or more of the above substituents.

The number of substituents is not particularly limited, and for example, may be 1 to 4.

As the divalent linking group represented by Sp, a linear alkylene group having 1 to 30 carbon atoms; a linking group in which a linear alkylene group having 1 to 30 carbon atoms and a cycloalkylene group having 3 to 10 carbon atoms are bonded to each other through a single bond, —O—, —C(═O)—, —OC(═O)—, —C(═O)O—, —OC(═O)O—, —NR⁴⁰¹C(═O)—, or —C(═O)NR⁴⁰²—; or a group in which one or two or more non-adjacent —CH₂—’s in a linear alkylene group having 2 to 30 carbon atoms are each independently substituted by a group selected from —O—, —S—, —C(═O)—, —OC(═O)—, —C(═O)O—, —OC(═O)O—, —NR⁴⁰¹C(—O)—, —C(═O)NR⁴⁰²—, —OC(═O)NR⁴⁰³—, —NR⁴⁰⁴C(═O)O—, —SC(═O)—, and —C(═O)S— is preferable.

In the above-described group in which —CH₂— in a linear alkylene group having 2 to 30 carbon atoms is substituted by a group selected from —O—, —S—, —C(═O)—, —OC(═O)—, —C(═O)O—, —OC(═O)O—, —NR⁴⁰¹C(—O)—, —C(═O)NR⁴⁰²—, —OC(═O)NR⁴⁰³—, —NR⁴⁰⁴C(═O)O—, —SC(═O)—, and —C(═O)S— (hereinafter, abbreviated as “other divalent groups” in this paragraph), it is preferable that the other divalent groups are not directly bonded to L. That is, it is preferable that a moiety substituted by the other divalent groups described above is not an L-side terminal in Sp.

As the divalent linking group represented by Sp, a linear alkylene group having 1 to 20 carbon atoms; a linking group in which a linear alkylene group having 1 to 20 carbon atoms and a cycloalkylene group having 3 to 6 carbon atoms are bonded to each other through —O—, —C(═O)—, —OC(═O)—, —C(═O)O—, or —OC(═O)O—; or a group in which one or two or more non-adjacent —CH₂—′s in a linear alkylene group having 2 to 20 carbon atoms are each independently substituted by a group selected from —O—, —C(═O)—, —OC(═O)—, —C(═O)O—, —OC(═O)O—, —NR⁴⁰¹C(—O)—, —C(═O)NR⁴⁰²—, —OC(═O)NR⁴⁰³—, and —NR⁴⁰⁴C(═O)O— is more preferable, a linear alkylene group having 1 to 10 carbon atoms; a linking group in which a linear alkylene group having 1 to 10 carbon atoms and a cycloalkylene group having 3 to 6 carbon atoms are bonded to each other through —O—, —C(═O)—, —OC(═O)—, or —C(═O)O—; or a group in which one or two or more non-adjacent —CH₂—′s in a linear alkylene group having 2 to 10 carbon atoms are each independently substituted by a group selected from —O—, —C(═O)—, —OC(═O)—, and —C(═O)O— is still more preferable, and a linear alkylene group having 1 to 10 carbon atoms, which is unsubstituted or has a methyl group as a substituent; a linking group in which a linear alkylene group having 1 to 10 carbon atoms, which is unsubstituted or has a methyl group as a substituent, and an unsubstituted cycloalkylene group having 3 to 6 carbon atoms are bonded to each other through —O—, —C(═O)—, —OC(═O)—, or —C(═O)O—; or a group in which one or two or more non-adjacent —CH₂—′s in a linear alkylene group having 2 to 10 carbon atoms, which is unsubstituted or has a methyl group as a substituent, are each independently substituted by a group selected from —O—, —C(═O)—, —OC(═O)—, and —C(═O)O— is particularly preferable.

In a case where n is 2, a plurality of Sp’s may be the same or different from each other, but it is preferable that Sp’s are the same.

In Pol—Sp—L—, it is preferable that Sp and L are not a single bond at the same time, and it is more preferable that neither of them is a single bond.

In General Formula (A), —L—Sp— is preferably a structure including —OC(═O)—C₂H₄— or —OC(═O)—C₂H₄—C(═O)O—C₂H₄— on an L-terminal side, more preferably a structure including —OC(═O)—C₂H₄—C(═O)O—C₂H₄— on an L-terminal side, and still more preferably —OC(═O)—C₂H₄—C(═O)O—C₂H₄—.

As the divalent linking group represented by Sp^{a}, a single bond or a linear alkylene group having 1 to 10 carbon atoms is preferable, a linear alkylene group having 1 to 5 carbon atoms is more preferable, a linear alkylene group having 1 to 3 carbon atoms is still more preferable, and an unsubstituted linear alkylene group having 1 to 3 carbon atoms is particularly preferable.

### Pol and Pol⁴

Pol and Pol⁴ represent a hydrogen atom or a polymerizable group.

The polymerizable group which can be adopted as Pol or Pol⁴ has the same meaning as the above-described polymerizable group.

Pol is preferably a polymerizable group, and more preferably a (meth)acryloyloxy group. In particular, from the viewpoint of improving moisture-heat resistance of the cured product obtained from the curable resin composition according to the embodiment of the present invention, Pol is more preferably a methacryloyloxy group.

In a case of a plurality of Pol’s, the plurality of Pol’s may be the same or different from each other, but it is preferable that the plurality of Pol’s are the same.

The compound represented by General Formula (A) has at least one polymerizable group. The compound represented by General Formula (A) preferably has at least two polymerizable groups. The upper limit value of the number of polymerizable groups included in the compound represented by General Formula (A) is not particularly limited, but for example, is preferably 4 or less.

The compound represented by General Formula (A) preferably has at least a polymerizable group as Pol, and more preferably has a polymerizable group only as Pol.

Pol⁴ is preferably a hydrogen atom.

As -Sp^{d}-Pol⁴, a hydrogen atom or an alkyl group having 1 to 4 carbon atoms is preferable, and a hydrogen atom or an unsubstituted alkyl group having 1 to 4 carbon atoms is more preferable.

In the compound represented by General Formula (A), in a case of a plurality of Pol—Sp—L—’s, the plurality of Pol—Sp—L—’s may be the same or different from each other, but it is preferable that the plurality of Pol—Sp—L—’s are the same.

Examples of a specific structure of Pol—Sp—L— include the following structures.

In the following structural examples, R represents a hydrogen atom or a methyl group. In addition, * represents a bonding position with Ar.

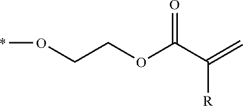

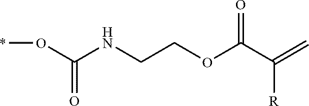

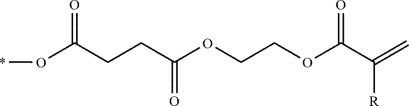

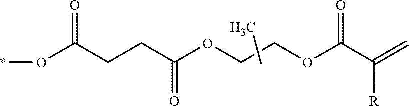

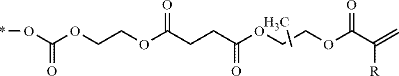

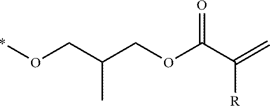

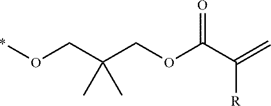

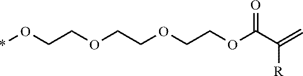

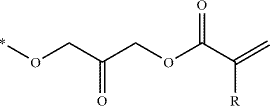

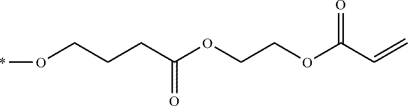 [SMILES: *OCCCC(=O)OCCOC(=O)C=C]

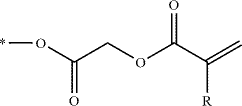

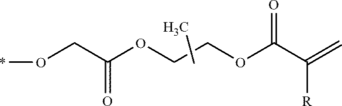

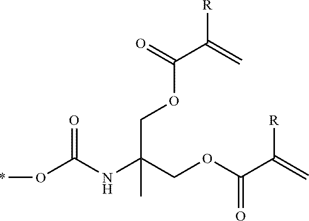

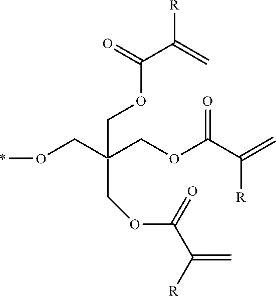

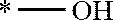 [SMILES: *O]

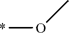 [SMILES: *OC]

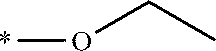 [SMILES: *OCC]

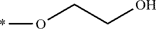 [SMILES: *OCCO]

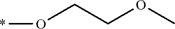 [SMILES: *OCCOC]

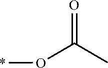 [SMILES: *OC(C)=O]

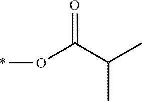 [SMILES: *OC(=O)C(C)C]

The compound represented by General Formula (A) is preferably a non-liquid crystalline compound.

Hereinafter, specific examples of the compound represented by General Formula (A), which is preferably used in the curable resin composition according to the embodiment of the present invention, will be shown, but the present invention is not limited to the following compounds.

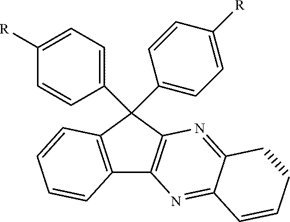

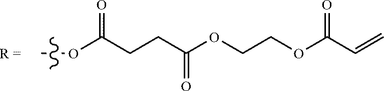

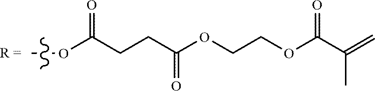

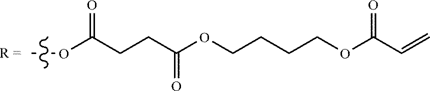

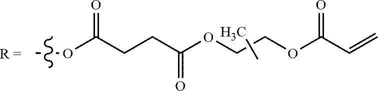

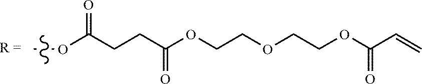

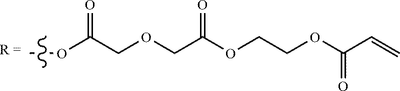

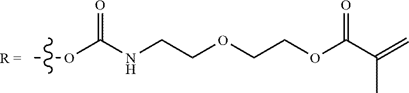

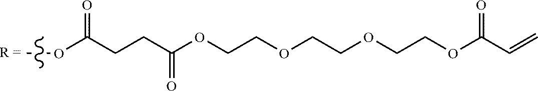

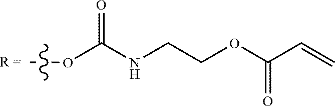

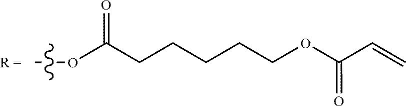

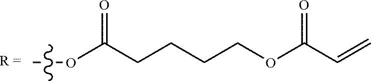

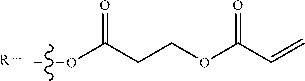

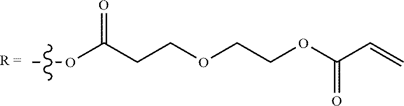

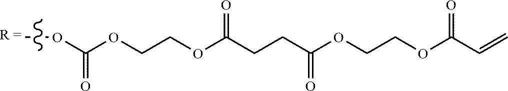

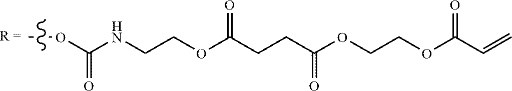

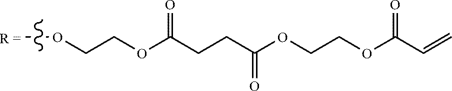

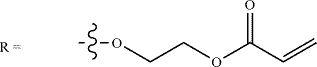

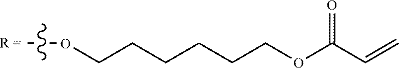

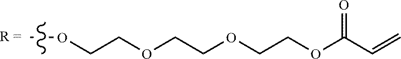

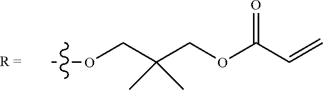

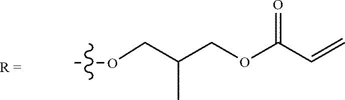

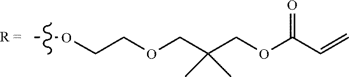

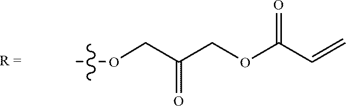

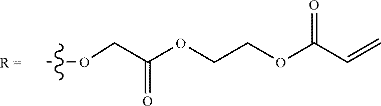

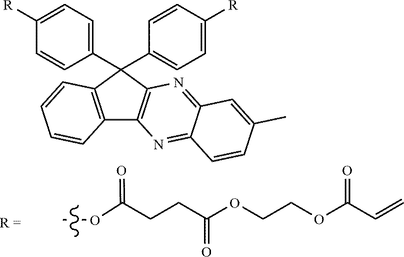

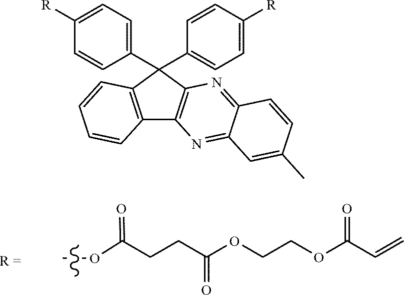

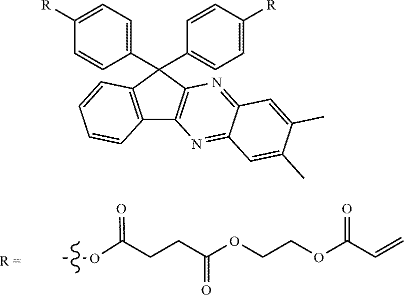

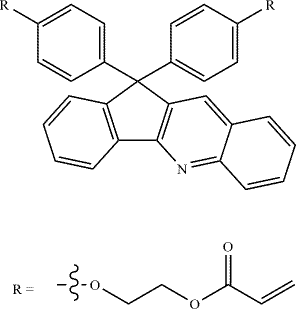

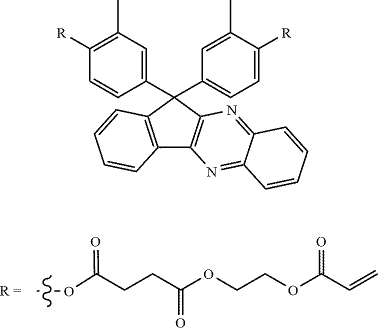

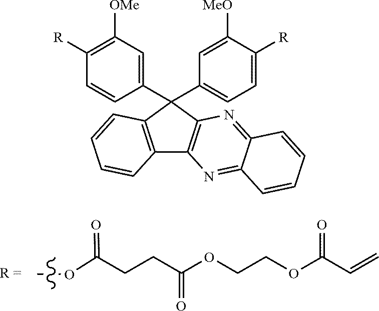

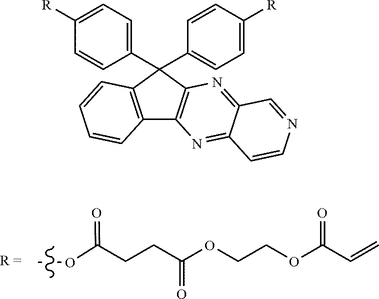

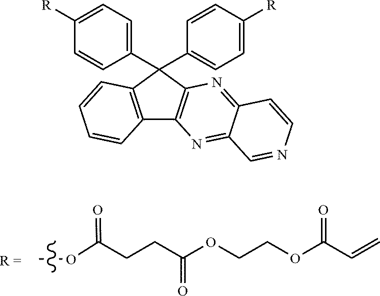

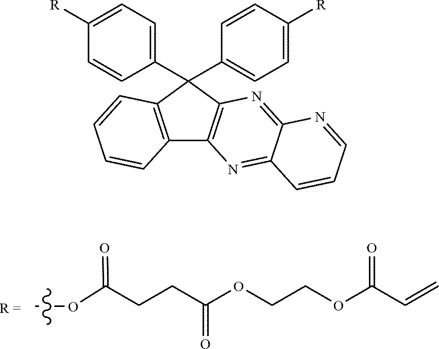

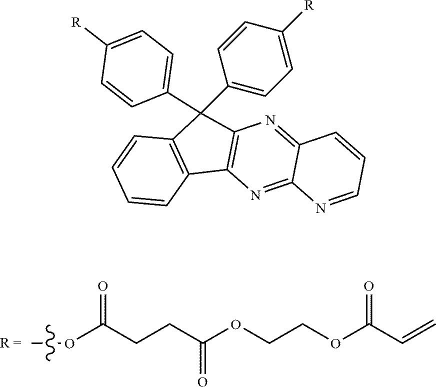

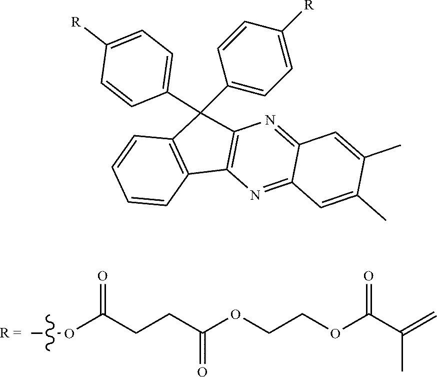

### Compound 3

The compound 3 preferred as the above-described near-ultraviolet light-absorbing organic compound is a compound represented by General Formula (B).

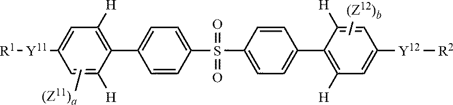

In the above formula, a and b are an integer of 1 or 2, and in consideration of ease of synthesis, a and b are preferably 1.

Y¹¹ and Y¹² represent —S— or —O—, and in consideration of ease of raw material procurement, Y¹¹ and Y¹² are preferably —O—.

R¹ and R² represent a hydrogen atom, a methyl group, or an ethyl group, and a methyl group or an ethyl group is preferable.

Z¹¹ and Z¹² represent a methyl group or an ethyl group having a substituent represented by General Formula (Z).

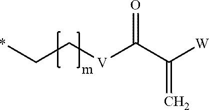

In the above formula, m is an integer of 0 or 1, and is preferably 0.

W represents a hydrogen atom or a methyl group.

V represents —O—CₙH₂ₙ—O—**, —S—CₙH₂ₙ—S—∗∗, or —S—CₙH₂ₙ—O—**. However, ** represents a bonding site with a (meth)acryloyl group represented by —C(═O)CW═CH₂. n is an integer of 2 to 4. However, at least one hydrogen atom in —CₙH₂ₙ— is substituted by a methyl group, and it is preferable that one or two hydrogen atoms in —CₙH₂ₙ— are substituted by methyl groups.

V is preferably —O—CₙH₂ₙ—O—**, and more preferably —O—CH(CH₃)—CH₂—O—**, —O—CH₂—CH(CH₃)—O—O—CₙH₂ₙ, —O—CH₂—CH(CH₃)—CH₂—O— or —O—CH₂—C(CH₃)₂—CH₂—O—.

Hereinafter, specific examples of the compound represented by General Formula (B), which is preferably used in the curable resin composition according to the embodiment of the present invention, will be shown, but the present invention is not limited to the following compounds.

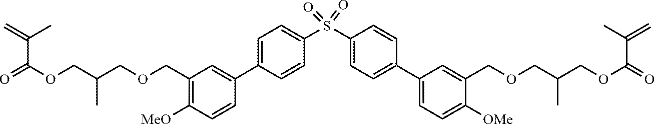

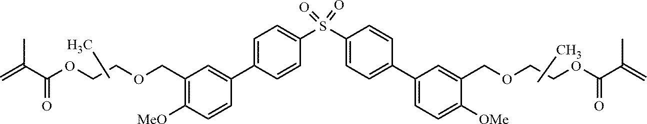

A method for obtaining the above-described compounds 1 to 3 is not particularly limited, and a commercially available product may be used or a compound obtained by synthesis may be used. In a case of being obtained by synthesis, a method for producing the compounds 1 to 3 is not particularly limited, and the compounds 1 to 3 can be produced according to a conventional method with reference to the method described in Examples later, and the like.

### Content and the Like of Near-Ultraviolet Light-Absorbing Organic Compound

It is sufficient that a content of the near-ultraviolet light-absorbing organic compound in the curable resin composition is adjusted according to the above-described A_{λmax} value of the near-ultraviolet light-absorbing organic compound and whether or not the near-ultraviolet light-absorbing organic compound is a polymerizable compound. Typically, the content of the near-ultraviolet light-absorbing organic compound in the curable resin composition is preferably 1% to 70% by mass, more preferably 5% to 60% by mass, still more preferably 10% by mass to 50% by mass, and particularly preferably 20% to 50% by mass. In a case where the content of the near-ultraviolet light-absorbing organic compound is within the above-described preferred range, an effect of increasing the refractive index in the near-ultraviolet light region can be sufficiently obtained.

Two or more kinds of near-ultraviolet light-absorbing organic compounds may be contained in the curable resin composition. In a case of containing two or more kinds of near-ultraviolet light-absorbing organic compounds, the total content is preferably within the above-described range.

### Indium Tin Oxide Particles

The curable resin composition according to the embodiment of the present invention includes indium tin oxide (also abbreviated as “ITO” in the present invention) particles. By adding ITO particles to the curable resin composition, it is possible to obtain a cured product having a lower refractive index as the wavelength in the visible light region is longer.

A particle diameter of the ITO particles is preferably 5 to 50 nm. By setting the particle diameter to 50 nm or less, it is possible to prevent a decrease in transmittance due to Rayleigh scattering. In addition, by setting the particle diameter to 5 nm or more, it is possible to perform a production of the ITO particles without technical difficulty. The particle diameter of the ITO particles can be obtained by averaging particle diameters which are measured by a transmission electron microscope (TEM). That is, a minor axis and a major axis of one particle in an electron micrograph imaged by TEM are measured, and the average value thereof is determined as a particle diameter of one particle. In the present invention, particle diameters of 500 particles are randomly obtained, and the average value (arithmetic mean) of these 500 particle diameters is calculated and used as an average primary particle diameter (particle diameter of the ITO particles).

The curable resin composition according to the embodiment of the present invention is preferably prepared by mixing ITO particles dispersed in a solvent with the above-described near-ultraviolet light-absorbing organic compound and a polymer (dispersant) described later. After mixing, the solvent used for dispersing the ITO particles may or may not be removed from the curable resin composition by distillation or the like, but it is preferable to be removed.

The dispersibility of the ITO particles in a solvent can be improved by using surface-modified ITO particles. The surface modification of the ITO particles is preferably performed using, for example, a monocarboxylic acid having 6 to 20 carbon atoms as a surface-modifying compound. As the surface modification of the ITO particles with a monocarboxylic acid, it is preferable that a carboxy group derived from the monocarboxylic acid forms an ester bond with an oxygen atom on the surface of the ITO particles, or the carboxy group is coordinated with In or Ti atom.

Examples of the monocarboxylic acid having 6 to 20 carbon atoms include oleic acid (having 18 carbon atoms), stearic acid (having 18 carbon atoms), palmitic acid (having 16 carbon atoms), myristic acid (having 14 carbon atoms), and decanoic acid (having 10 carbon atoms), and oleic acid (having 18 carbon atoms) is preferable.

In the curable resin composition, a moiety derived from the surface-modifying compound in the ITO particles (for example, a group derived from a monocarboxylic acid having 6 to 20 carbon atoms) bonded to the ITO particles by the above-described surface modification may be bonded to the ITO particles as it is, a part thereof may be substituted by a group derived from a polymer described later, or all may be substituted by groups derived from a polymer described later. In the curable resin composition according to the embodiment of the present invention, it is preferable that both the moiety derived from the surface-modifying compound (for example, a group derived from a monocarboxylic acid having 6 to 20 carbon atoms) and the group derived from the polymer described later are bonded to the surface of the ITO particles.

As the solvent, a solvent, in which a constituent (δp) of a polarity element in a solubility parameter (SP value) is 0 to 6 MPa^{(½)}, is preferable.

The constituent (δp) of the polarity element in the SP value is a value calculated by the Hansen solubility parameter. The Hansen solubility parameter is constituted of intermolecular dispersive force energy (δd), intermolecular polar energy (δp), and intermolecular hydrogen bonding energy (δh). In the present invention, the Hansen solubility parameter is a value calculated using HSPiP (version 4.1.07) software.

Specifically, the solvent is preferably toluene (1.4), xylene (1.0), or hexane (0), and more preferably toluene. The value in the parentheses is a value of δp, and the unit is MPa^{(½)}.

A method for producing the ITO particles is not particularly limited, and for example, the ITO particles can be produced according to the procedure described in ACS Nano 2016, 10, pp. 6942 to 6951. According to the procedure of the reference, a dispersion liquid of surface-modified ITO particles is obtained.

Specifically, a solution obtained by mixing a monocarboxylic acid having 6 to 20 carbon atoms, an indium salt (for example, indium acetate), and a tin salt (for example, tin acetate) is added dropwise to an alcohol (long-chain alcohol such as oleyl alcohol) heated to high temperature, and the mixture is retained at high temperature, thereby capable of forming particles.

Thereafter, a poor solvent (lower alcohol such as ethanol) having low polymer solubility is added thereto to precipitate the particles, the supernatant is removed, and the particles are redispersed in the above-described solvent such as toluene, thereby capable of forming a dispersion liquid of surface-modified ITO particles.

A content proportion of the ITO particles in the curable resin composition according to the embodiment of the present invention is preferably 10% to 70% by mass, more preferably 10% to 60% by mass, and still more preferably 20% to 50% by mass.

### Polymer (Dispersant)

The polymer included in the composition according to the embodiment of the present invention functions as a dispersant in the curable resin composition (in the present invention, this polymer is also referred to as a “polymer dispersant”). The polymer dispersant has a constitutional unit represented by General Formula (P) and also has an acidic group at one terminal of a polymer chain.

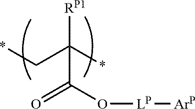

In the formula, L^{p} represents a single bond or a divalent linking group, Ar^{P} represents an aryl group, and R^{P1} represents a hydrogen atom or a methyl group. However, Ar^{P} does not include the acidic group. * represents a bonding portion for incorporation into a polymer main chain.

As the aryl group of Ar^{P}, a phenyl group, a 1-naphthyl group, or a 2-naphthyl group is preferable. Preferred examples of the substituent which may be included in the aryl group include an alkyl group, an alkoxy group, and an aryl group.

It is preferable that the methyl group which can be adopted as R^{P1} does not include the above-described acidic group as a substituent.

The above-described polymer dispersant is a polymer which has an acidic group exhibiting an adsorbing group for the ITO particles at one terminal of the polymer chain and also has the constitutional unit represented General Formula (P) including Ar^{P} (aryl group). Since the curable resin composition according to the embodiment of the present invention contains the above-described polymer dispersant together with the ITO particles and the near-ultraviolet light-absorbing organic compound, it is considered that a compatibility of components increases due to a π-π interaction between Ar^{P} included in the side chain of the polymer dispersant and the aromatic ring included in the near-ultraviolet light-absorbing organic compound, an interaction between the acidic group of the polymer dispersant and the ITO particles, and the like, so that a dispersion stability of the composition is effectively enhanced. Since the curable resin composition according to the embodiment of the present invention contains the above-described polymer dispersant, it is possible to enhance a dispersibility of the curable resin composition during preparation and also possible to sufficiently enhance a medium- to long-term dispersion stability.

As the acidic group included in the polymer dispersant at one terminal of the polymer chain, it is preferable to select from a carboxy group (—COOH), a phosphono group (—P(═O)(OH)₂), a phosphonooxy group (—OP(═O)(OH)₂), a hydrohydroxyphosphoryl group (—PH(═O)(OH)), a sulfino group (—S(═O)(OH)), a sulfo group (—S(═O)₂(OH)), or a sulfanyl group (—SH).

The other terminal of the polymer chain in the above-described polymer dispersant is not particularly limited as long as the effects of the present invention are exhibited, but it is preferable that the other terminal thereof does not have an acidic group, and the other terminal thereof can be, for example, a hydrogen atom, an alkyl group, or the like.

For convenience of synthesis, the above-described polymer dispersant may include a small amount of a polymer having acidic groups at both terminals of the polymer chain, in addition to the polymer having an acidic group at one terminal of the polymer chain. However, as long as the above-described polymer dispersant is substantially composed of the polymer having an acidic group at one terminal of the polymer chain, even in a case where the above-described polymer having acidic groups at both terminals is included, the effects of the present invention can be exhibited.

In addition, the above-described polymer dispersant may include an acidic group in the side chain of the polymer chain as long as the effects of the present invention can be exhibited. However, in a case where the side chain includes an acidic group, it is preferable not to include the acidic group because the ITO particles tend to aggregate.

The above-described acidic group exhibits an adsorption action on a surface of the indium tin oxide particles by at least one of an ionic bond, a covalent bond, a hydrogen bond, or a coordinate bond.

From the viewpoint of further improving the medium- to long-term dispersion stability, the above-described acidic group is more preferably a carboxy group, a phosphono group, or a phosphonooxy group, and still more preferably a carboxy group.

In General Formula (P), examples of the divalent linking group which can be adopted as L^{P} include an alkylene group, ∗—(alkylene—O)ₙ—, and ester (—O—(C═O)—). The number of carbon atoms in the alkylene moiety is preferably 1 to 4 and more preferably 1 or 2. n is preferably 1 to 10, more preferably 1 to 6, still more preferably 1 or 2, and particularly preferably 1.

L^{P} is preferably a single bond, an alkylene group, or ^{∗}—(alkylene—O)ₙ—, and more preferably a single bond, —CH₂—, *—CH₂O—, or ^{∗}—CH₂CH₂O—.

* in the above description of L^{P} represents a bonding site on a side which does not bond with Ar^{P}.

A main chain skeleton portion of the above-described polymer dispersant may be linear or branched. Among these, it is preferable to be linear.

As long as the effects of the present invention can be exhibited, the above-described polymer dispersant may have a constitutional unit represented by General Formula (P2) in addition to the constitutional unit represented by General Formula (P).

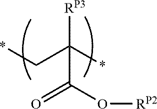

In the formula, R^{P3} represents a hydrogen atom or a methyl group, and R^{P2} represents a monovalent substituent. However, R^{P2} is not —L^{P}—Ar^{P} in General Formula (P) described above. * represents a bonding portion for incorporation into a polymer main chain.

R^{P2} is preferably an alkyl group or an alicyclic hydrocarbon group, and more preferably an alkyl group. From the viewpoint of suppressing aggregation of the ITO particles, the monovalent substituent which can be adopted as R^{P2} preferably does not include the above-described acidic group. The number of carbon atoms in this alkyl group is preferably 1 to 20, more preferably 1 to 12, and still more preferably 1 to 8.

It is preferable that the methyl group which can be adopted as R^{P3} does not include the above-described acidic group as a substituent.

In the above-described polymer dispersant, it is preferable that the main chain structure and the side chain structure are composed of the constitutional unit represented by General Formula (P), and it is also preferable to be composed of the constitutional unit represented by General Formula (P) and the constitutional unit represented by General Formula (P2). In addition, as long as the effects of the present invention can be exhibited, the above-described polymer dispersant may have a constitutional unit different from the constitutional units represented by each of General Formulae (P) and (P2) (constitutional unit derived from a monomer having an ethylenically unsaturated bond, which is not the constitutional unit represented by each of General Formulae (P) and (P2)). In a case where the above-described polymer dispersant is a copolymer, it may be either random or block.

A proportion of General Formula (P) to all constitutional units constituting the above-described polymer dispersant is not particularly limited, but is preferably 5 mol% or more, for example. From the viewpoint of further improving the medium- to long-term dispersion stability, the above-described proportion is more preferably 10 mol% or more and still more preferably 15 mol% or more. The upper limit value of this proportion is not particularly limited, and it is also preferable that all constitutional units in the above-described polymer dispersant are the constitutional unit represented by General Formula (P).

In a case where the above-described polymer dispersant contains the constitutional unit represented by General Formula (P2), a proportion of General Formula (P2) to all constitutional units constituting the polymer dispersant is, for example, preferably 95 mol% or less, more preferably 90 mol% or less, and still more preferably 85 mol% or less. The lower limit value of the above-described proportion in a case of containing the constitutional unit represented by General Formula (P2) is not particularly limited, and may be more than 0 mol%.

The constitutional unit constituting the above-described polymer dispersant means a constitutional unit derived from a monomer component, and can be calculated from a content ratio of the monomer component.

The content of the constitutional unit represented by General Formula (P) in the above-described polymer dispersant is not particularly limited, but is preferably, for example, 20% by mass or more. From the viewpoint of further improving the medium- to long-term dispersion stability, the above-described content is more preferably 30% by mass or more and still more preferably 50% by mass or more. The upper limit value of this content is not particularly limited, and it is also preferable that all constitutional units in the above-described polymer dispersant are the constitutional unit represented by General Formula (P).

The above-described polymer dispersant preferably has, at one terminal of the polymer chain, a structural portion represented by General Formula (PA) as a structural portion including the above-described acidic group.

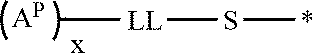

In the formula, A^{P} represents an acidic group, LL represents a single bond or an (x+1)-valent linking group, in which x represents an integer of 1 to 8. * represents a bonding position with the remaining moiety of the polymer dispersant.

The acidic group which can be adopted as A^{P} has the same meaning as the acidic group described above, and the preferred form thereof is also the same.

Examples of the (x+1)-valent linking group which can be adopted as LL include an (x+1)-valent saturated fatty acid hydrocarbon group (group obtained by removing x+1 hydrogen atoms from alkane) and an (x+1)-valent alicyclic hydrocarbon group (group obtained by removing x+1 hydrogen atoms from alicyclic hydrocarbon). In addition, examples thereof include an (x+1)-valent group consisting of a combination of these groups and a bond selected from —O—, —(C═O)—O—, or —(C═O)—NH—. LL is preferably an (x+1)-valent alkane or a group consisting of a combination of an (x+1)-valent alkane and —O—.

x is preferably an integer of 1 to 6, more preferably an integer of 2 to 4, and still more preferably an integer of 2.

The structure represented by General Formula (PA) is preferably a structure represented by General Formula (PA1), and from the viewpoint of improving the adsorptivity to the ITO particles by having a carboxy group in the adjacent site, more preferably a structure represented by Formula (PA2).

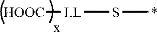

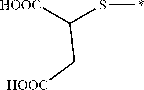

LL and x in the formulae have the same meaning as LL and x in General Formula (PA) described above. * represents a bonding position with the remaining moiety of the polymer dispersant.

An acid value of the above-described polymer dispersant is preferably 2.0 mgKOH/g or more and less than 100 mgKOH/g, more preferably 2.0 mgKOH/g or more and less than 70 mgKOH/g, and still more preferably 10 mgKOH/g or more and less than 50 mgKOH/g. The acid value means the number in mg of potassium hydroxide required to neutralize acid components present in 1 g of the polymer.

By adjusting the molecular weight of the polymer dispersant and the number of acidic groups such as a carboxy group so that the acid value of the polymer dispersant is within the above-described preferred range, it is possible to achieve both appropriate viscosity and particle dispersion performance as the curable resin composition. In a case where the acid value of the polymer dispersant is 2.0 mgKOH/g or more, the polymer dispersant can be sufficiently adsorbed and dispersed on the ITO particles. In addition, in a case where the acid value of the polymer dispersant is less than the above-described preferred upper limit value, the number and the molecular size of adsorptive groups can be adjusted to adjust the viscosity of the curable resin composition to an appropriate range.

A weight-average molecular weight of the above-described polymer dispersant is not particularly limited, but for example, is preferably 1000 to 30000, and from the viewpoint of further improving the medium- to long-term dispersion stability, more preferably 1000 to 20000, still more preferably 1000 to 15000, and particularly preferably 1000 to 13000. By setting the weight-average molecular weight of the polymer dispersant to 1000 or more, it is possible to suppress mixing of bubbles generated during curing the curable resin composition. In addition, by setting the weight-average molecular weight of the polymer dispersant to the above-described preferred upper limit value or less, the fluidity is less likely to decrease even in a case where an amount necessary for dispersing the ITO particles is added to the curable resin composition, and even in a case of forming a cured product having a diffraction grating shape, air gaps are unlikely to occur in a level difference of the mold.

The weight-average molecular weight of the polymer dispersant is a value measured by a method described in Examples described later.

Specific examples of the above-described polymer dispersant are listed below, the structure thereof is not limited to these. Although the specific examples shown below are all homopolymers, the above-described polymer dispersant may be a copolymer and may have a constitutional unit other than the constitutional unit represented by General Formula (P). In addition, the specific examples shown below have a structural portion including an acidic group at one terminal, and the other terminal is a methyl group, but a group other than the methyl group may be used. n has the same meaning as n in L^{p} of General Formula (P) described above.

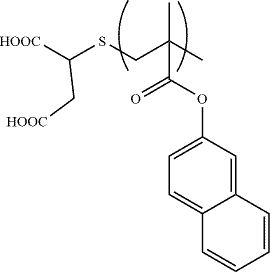

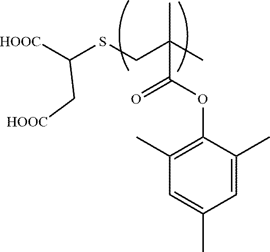

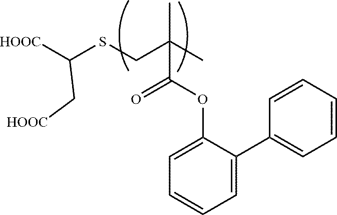

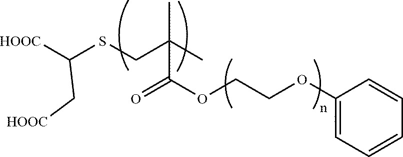

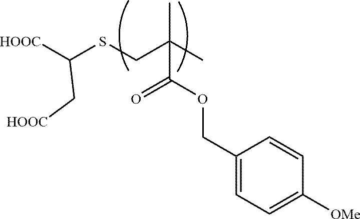

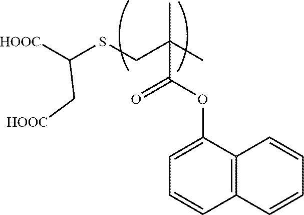

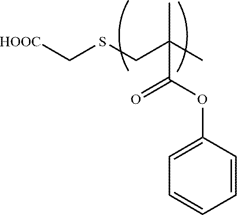

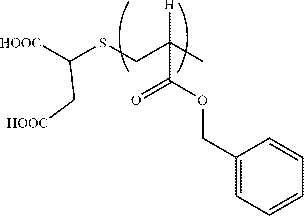

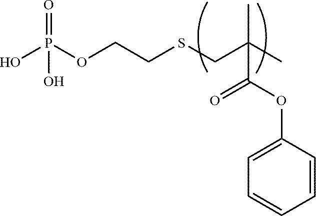

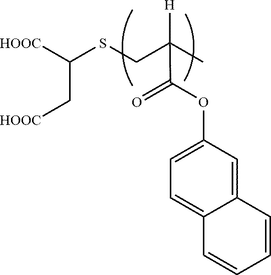

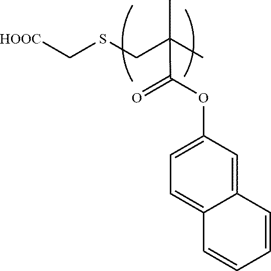

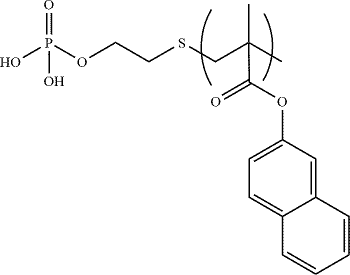

The above-described polymer dispersant can be produced by a conventional method. For example, the polymer dispersant can be produced by a reaction between a (meth)acrylate monomer and a compound capable of terminating the polymerization reaction of this monomer and having an acidic group (preferably, a carboxyl group). Examples of such compounds include mercaptosuccinic acid, mercaptooxalic acid, and mercaptomalonic acid, and mercaptosuccinic acid is preferable. In addition, with regard to a polymer dispersant having a phosphonooxy group at one terminal, a method described in JP1994-20261A (JP-H6-20261A) can be referred to.

In the curable resin composition according to the embodiment of the present invention, a content of the polymer dispersant is preferably 1 to 50 parts by mass, more preferably 3 to 30 parts by mass, and still more preferably 4 to 30 parts by mass with respect to 100 parts by mass of the content of the ITO particles. By setting the content ratio to the above-described preferred range, it is possible to suppress the mixing of bubbles generated during curing while stably dispersing the ITO particles in the curable resin composition.

### Other Components

The curable resin composition according to the embodiment of the present invention may further include other components in addition to the near-ultraviolet light-absorbing organic compound, ITO particles, and polymer dispersant. Specific examples of the other components include at least one selected from a (meth)acrylate monomer compound, a polymer, a photoradical polymerization initiator, and a thermal radical polymerization initiator.

### (Meth)acrylate Monomer Compound

The curable resin composition according to the embodiment of the present invention may include a (meth)acrylate monomer compound. The (meth)acrylate monomer compound may be a polyfunctional (meth)acrylate monomer compound having two or more (meth)acryloyl groups in the molecule, or may be a monofunctional (meth)acrylate monomer compound having one (meth)acryloyl group in the molecule.

Specific examples of the (meth)acrylate monomer compound include the following monomer 1 (phenoxyethyl acrylate), monomer 2 (benzyl acrylate), monomer 3 (tricyclodecanedimethanol diacrylate), and monomer 4 (dicyclopentanyl acrylate). In addition, specific examples thereof include M-1 (1,6-hexanediol diacrylate), M-2 (1,6-hexanediol dimethacrylate), M-3 (benzyl acrylate), M-4 (isobomyl methacrylate), M-5 (dicyclopentanyl methacrylate), M-6 (dodecyl methacrylate), M-7 (2-ethylhexyl methacrylate), M-8 (2-hydroxyethyl acrylate), M-9 (hydroxypropyl acrylate), and M-10 (4-hydroxybutyl acrylate). In addition to the above, examples thereof include (meth)acrylate monomers described in paragraphs 0037 to 0046 of JP2012-107191A.

A molecular weight of the (meth)acrylate monomer compound is preferably 100 to 500.

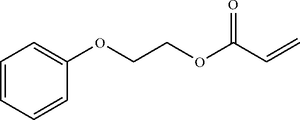

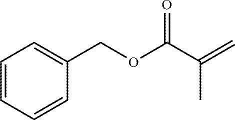

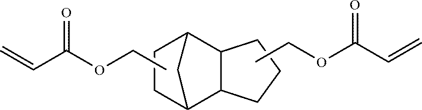

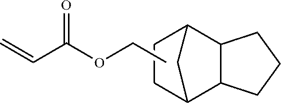

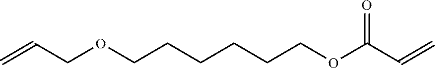

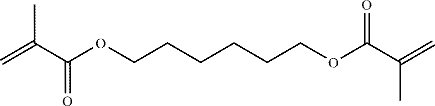 [SMILES: C=C(C)C(=O)OCCCCCCOC(=O)C(=C)C]

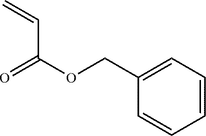

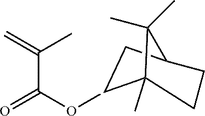

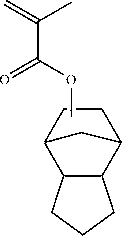

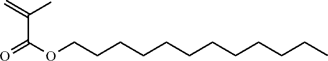 [SMILES: C=C(C)C(=O)OCCCCCCCCCCCC]

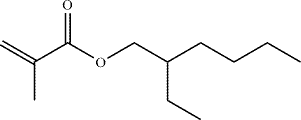

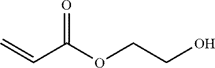

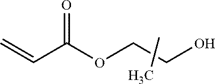

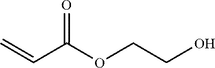

A method for obtaining the (meth)acrylate monomer compound is not particularly limited, and the (meth)acrylate compound may be obtained commercially or may be synthesized by a conventional method.

In a case of being obtained commercially, for example, Viscoat#192 PEA (monomer 1 described above) (manufactured by OSAKA ORGANIC CHEMICAL INDUSTRY LTD.), Viscoat#160 BZA (monomer 2 described above) (manufactured by OSAKA ORGANIC CHEMICAL INDUSTRY LTD.), Lightester Bz (monomer 2 described above) (manufactured by KYOEISHA CHEMICAL Co., LTD.), A-DCP (monomer 3 described above) (manufactured by Shin-Nakamura Chemical Co., Ltd.), FA-513AS (monomer 4 described above) (manufactured by Hitachi Chemical Co., Ltd.), A-HD-N (M-1 described above) (manufactured by Shin-Nakamura Chemical Co., Ltd.), HD-N (M-2 described above) (manufactured by Shin-Nakamura Chemical Co., Ltd.), FA-BZA (M-3 described above) (manufactured by Hitachi Chemical Co., Ltd.), Lightester IB-X (M-4 described above) (manufactured by KYOEISHA CHEMICAL Co., LTD.), FA-513M (M-5 described above) (manufactured by Hitachi Chemical Co., Ltd.), Lightester L (M-6 described above) (manufactured by KYOEISHA CHEMICAL Co., LTD.), 2EHA (M-7 described above) (manufactured by TOAGOSEI CO., LTD.), HEA (M-8 described above) (manufactured by OSAKA ORGANIC CHEMICAL INDUSTRY LTD.), Lightester HOP-A(N) (M-9 described above) (manufactured by KYOEISHA CHEMICAL Co., LTD.), or 4-HBA (M-10 described above) (manufactured by OSAKA ORGANIC CHEMICAL INDUSTRY LTD.) can be preferably used.

In addition, in a case where it is necessary to increase the hardness and rub resistance of the surface of the cured product, the curable resin composition preferably includes a polyfunctional (meth)acrylate monomer compound having three or more (meth)acryloyl groups in the molecule. By including a polyfunctional (meth)acrylate monomer compound having three or more (meth)acryloyl groups in the molecule, crosslinking density of the cured product can be effectively improved, so that the surface hardness and rub resistance can be increased while maintaining a high partial dispersion ratio. The upper limit of the number of (meth)acryloyl groups in the polyfunctional (meth)acrylate monomer compound having three or more (meth)acryloyl groups in the molecule is not particularly limited, but is preferably 8 or less and more preferably 6 or less. In a case of being obtained commercially, for example, A-TMPT (monomer 5), A-TMMT (monomer 6), AD-TMP (monomer 7), and A-DPH (monomer 8) (all manufactured by Shin-Nakamura Chemical Co., Ltd.) can be preferably used. In addition, trimethylolpropane trimethacrylate in which all three acryloyl groups in the monomer 5 are substituted by methacryloyl groups can also be preferably used.

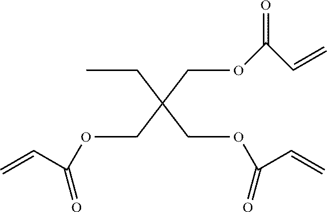 [SMILES: C=CC(=O)OCC(CC)(COC(=O)C=C)COC(=O)C=C]

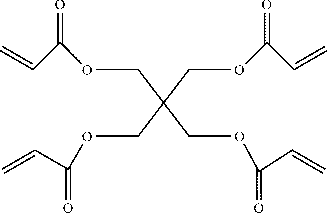 [SMILES: C=CC(=O)OCC(COC(=O)C=C)(COC(=O)C=C)COC(=O)C=C]

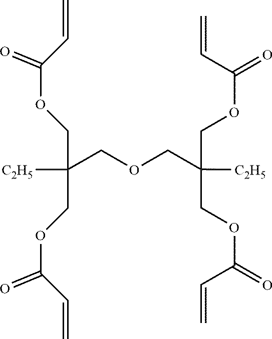 [SMILES: C=CC(=O)OCC(CC)(COCC(CC)(COC(=O)C=C)COC(=O)C=C)COC(=O)C=C]

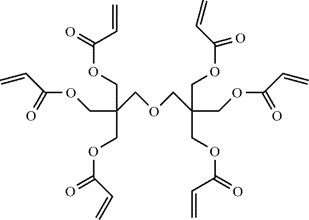 [SMILES: C=CC(=O)OCC(COCC(COC(=O)C=C)(COC(=O)C=C)COC(=O)C=C)(COC(=O)C=C)COC(=O)C=C]

In a case where the curable resin composition contains a (meth)acrylate monomer compound, a content of the (meth)acrylate monomer compound is preferably 1% to 50% by mass, more preferably 2% to 40% by mass, and still more preferably 3% to 30% by mass with respect to the total mass of the curable resin composition. The content of the (meth)acrylate monomer compound in the curable resin composition can be adjusted to adjust the function of the cured product to relax stress during thermal changes.

In particular, in a case where it is necessary to increase the surface hardness and rub resistance of the cured product, the curable resin composition includes the polyfunctional (meth)acrylate monomer compound having three or more (meth)acryloyl groups in the molecule in an amount of preferably 5% to 50% by mass, more preferably 10% to 45% by mass, and still more preferably 25% to 40% by mass with respect to the total mass (in a case of including a solvent, a mass of solid content excluding the solvent) of the curable resin composition.

### Polymer

The curable resin composition according to the embodiment of the present invention may further include a polymer in addition to the above-described compounds. In particular, a polymer having a radically polymerizable group has a function of increasing the viscosity of the curable resin composition, so that the polymer can also be called a thickener or a thickening polymer. The polymer can be added to adjust the viscosity of the curable resin composition. However, the polymer does not have to include a radically polymerizable group.

Examples of the polymer include a polymer having a radically polymerizable group in the side chain described later, a polyacrylic acid ester, a urethane oligomer, a polyester, and a polyalkylene. Examples of the polyacrylic acid ester include methyl polyacrylate and butyl polyacrylate. In addition, as the polymer, commercially available products such as LIR-30, 50, 290, 310, 390, and 700 (KURARAY CO., LTD.) can also be used.

### Polymer Having Radically Polymerizable Group

The polymer having a radically polymerizable group may be a homopolymer or a copolymer. It is more preferably a polymer in which a moiety having a radically polymerizable group is introduced into a side chain of polyacrylic acid ester, urethane oligomer, polyester, or polyalkylene.

Examples of the radically polymerizable group include a (meth)acrylate group, a vinyl group, a styryl group, and an allyl group. In the polymer having a radically polymerizable group in the side chain, a constitutional unit having a radically polymerizable group is included in an amount of preferably 5% to 100% by mass, more preferably 10% to 90% by mass, and still more preferably 20% to 80% by mass.

Hereinafter, specific examples of the polymer having a radically polymerizable group, which is preferably used in the present invention, will be shown, but the polymer having a radically polymerizable group is not limited to the following structures. Specific examples shown below are all copolymers and include two or three constitutional units described as being adjacent to each other. For example, a specific example described at the left end of the uppermost stage is a copolymer of allyl methacrylate and benzyl methacrylate.

In the following structural formulae, Ra and Rb each independently represent a hydrogen atom or a methyl group. In addition, n represents an integer of 0 to 10, and is preferably 0 to 2 and more preferably 0 or 1. An amount ratio of each constitutional unit in the copolymer is not particularly limited, and as the content of the constitutional unit having a radically polymerizable group in the copolymer, the above description can be preferably applied.

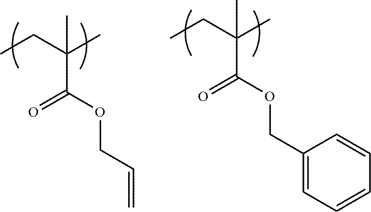

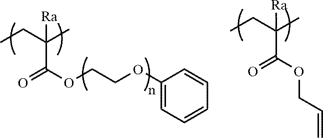

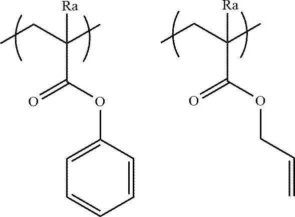

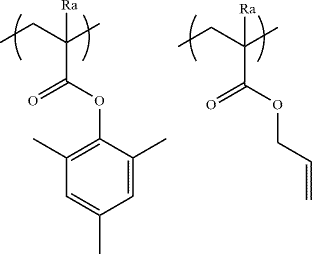

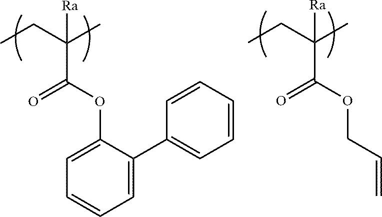

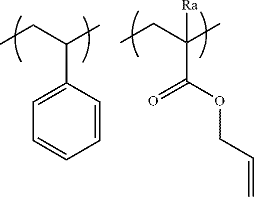

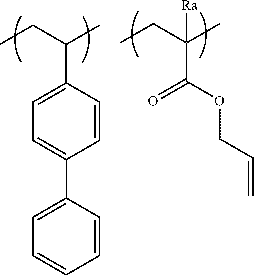

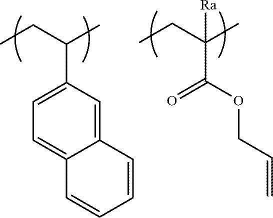

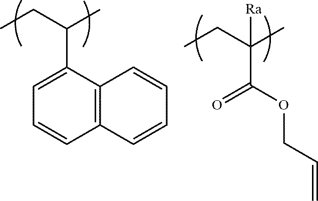

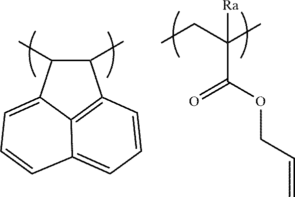

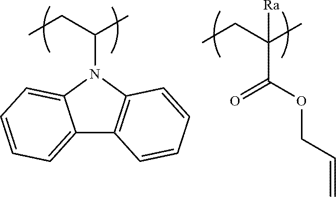

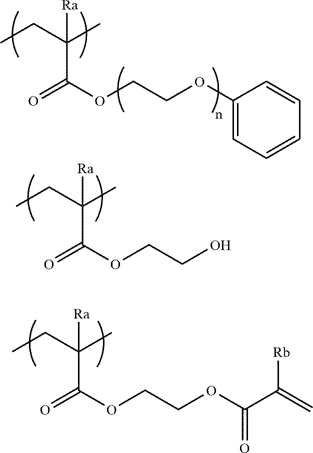

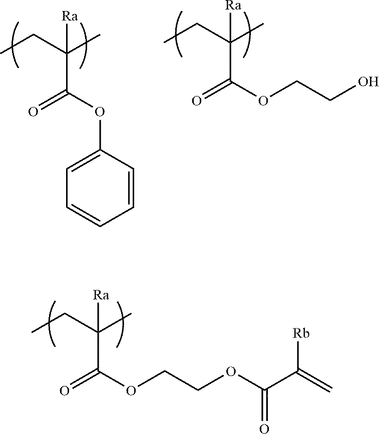

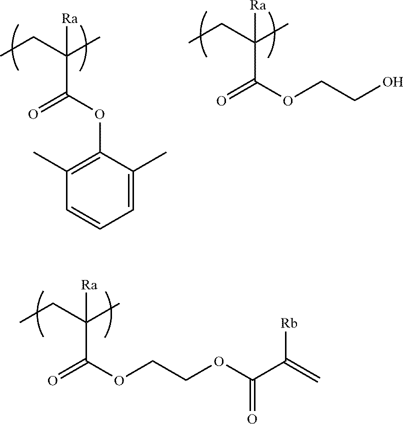

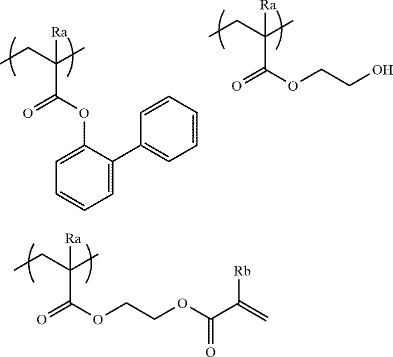

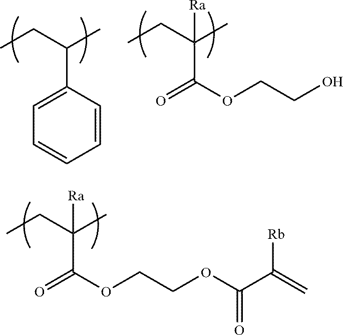

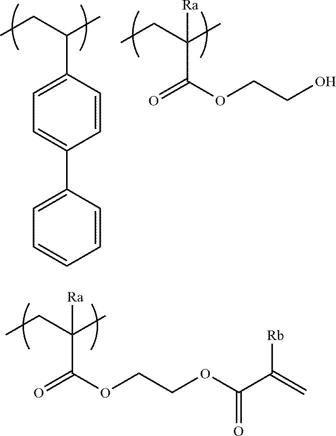

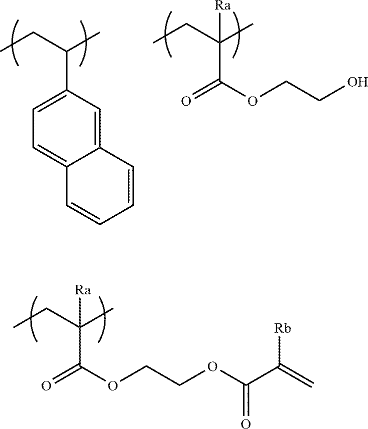

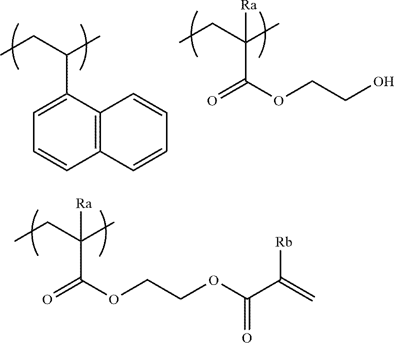

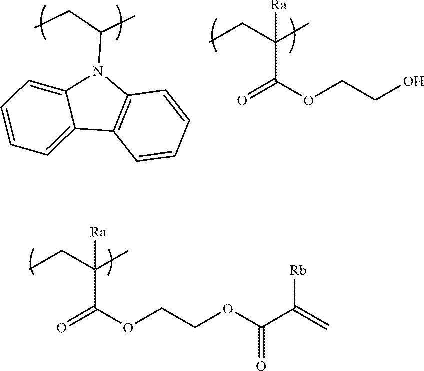

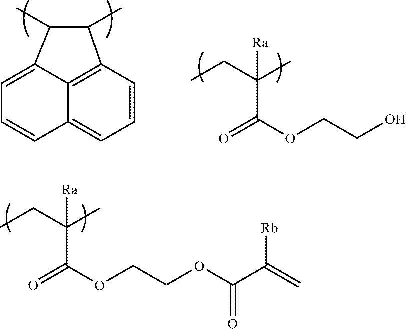

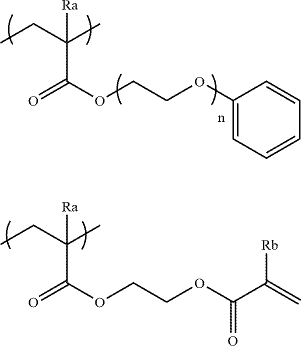

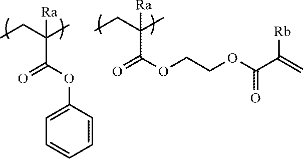

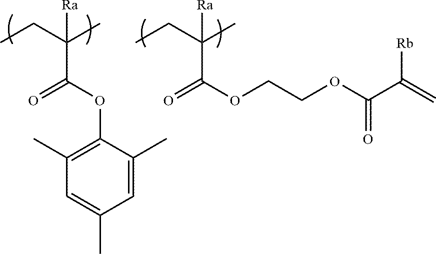

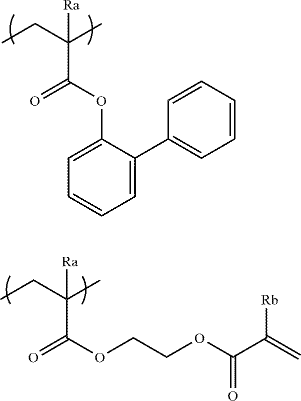

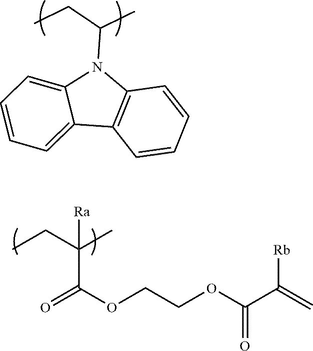

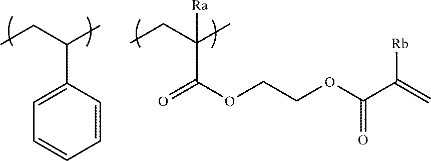

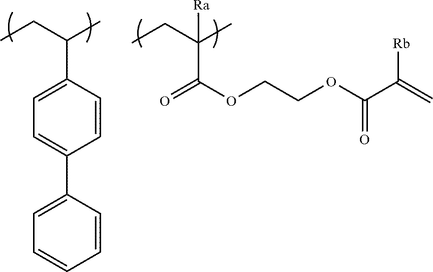

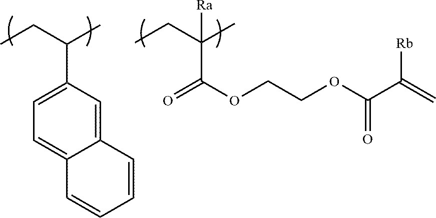

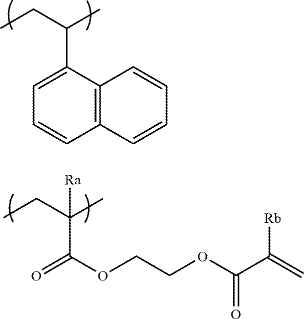

In addition, examples of a commercially available product thereof include UC-102M and 203M (KURARAY CO., LTD.), AA-6, AS-6S, and AB-6 (TOAGOSEI CO., LTD.), Shikou series (The Nippon Synthetic Chemical Industry Co., Ltd.), and EBECRYL270, 8301R, 8402, 8465, and 8804 (DAICEL-ALLNEX LTD.).

A molecular weight (weight-average molecular weight) of the polymer is preferably 1,000 to 10,000,000, more preferably 5,000 to 300,000, and still more preferably 10,000 to 200,000. In addition, a glass transition temperature of the polymer is preferably -50° C. to 400° C. and more preferably -30° C. to 350° C.

A content of the polymer in the curable resin composition is preferably 40% by mass or less, more preferably 30% by mass or less, and still more preferably 25% by mass or less. The content of the polymer may be 0% by mass, and an aspect in which no polymer is added is also preferable.

### Polymerization Initiator

The curable resin composition according to the embodiment of the present invention preferably includes at least one selected from a thermal radical polymerization initiator or a photoradical polymerization initiator.

### Thermal Radical Polymerization Initiator

The curable resin composition preferably includes a thermal radical polymerization initiator. By the action of this thermal radical polymerization initiator, a cured product having high heat resistance can be obtained by thermally polymerizing the curable resin composition.

As the thermal radical polymerization initiator, a compound usually used as a thermal radical polymerization initiator can be appropriately used according to conditions of a thermopolymerization (heat curing) step described later. Examples thereof include organic peroxides, and specifically, the following compounds can be used.

Examples thereof include 1,1-di(t-hexylperoxy) cyclohexane, 1,1-di(t-butylperoxy) cyclohexane, 2,2-di(4,4-di-(t-butylperoxy)cyclohexyl) propane, t-hexylperoxyisopropyl monocarbonate, t-butylperoxy-3,5,5-trimethylhexanoate, t-butylperoxylaurate, dicumyl peroxide, di-t-butyl peroxide, t-butylperoxy-2-ethylhexanoate, t-hexylperoxy-2-ethylhexanoate, cumene hydroperoxide, t-butyl hydroperoxide, t-butylperoxy-2-ethylhexyl, and 2,3-dimethyl-2,3-diphenylbutane.

In a case of including a thermal radical polymerization initiator, a content of the thermal radical polymerization initiator in the curable resin composition according to the embodiment of the present invention is preferably 0.01% to 10% by mass, more preferably 0.05% to 5.0% by mass, and still more preferably 0.05% to 2.0% by mass.

### Photoradical Polymerization Initiator

The curable resin composition preferably includes a photoradical polymerization initiator. As the photoradical polymerization initiator, a compound usually used as a photoradical polymerization initiator can be appropriately used according to conditions of a photopolymerization (photocuring) step described later, and specifically, the following compounds can be used.

Examples thereof include bis(2,6-dimethoxybenzoyl)-2,4,4-trimethylpentyl phosphine oxide, bis(2,6-dimethylbenzoyl)-2,4,4-trimethylpentyl phosphine oxide, bis(2,4,6-trimethylbenzoyl)-2,4,4-trimethylpentyl phosphine oxide, bis(2,6-dichlorobenzoyl)-2,4,4-trimethylpentyl phosphine oxide, 1-phenyl-2-hydroxy-2-methylpropan-1-one, 1-hydroxycyclohexylphenylketone, 1-(4-isopropylphenyl)-2-hydroxy-2-methylpropan-1-one, 1,2-diphenylethanedione, methylphenyl glyoxylate, 1-[4-(2-hydroxy ethoxy)-phenyl] -2-hydroxy-2-methyl-1 -propan-1 -one, 2-hydroxy-1-{4-[4-(2-hydroxy-2-methyl-propionyl)-benzyl]phenyl}-2-methyl-propan-1-one, 2,2-dimethoxy-1,2-diphenylethan-1-one, 2-methyl-1-(4-methylthiophenyl)-2-morpholinopropan-1-one, 2-benzyl-2-dimethylamino-1-(4-morpholinophenyl)-butanone-1,2,4,6-trimethylbenzoyl-diphe nyl-phosphine oxide, and bis(2,4,6-trimethylbenzoyl)-phenylphosphine oxide.

Among these, in the present invention, as the photoradical polymerization initiator, 1-hydroxycyclohexylphenylketone (for example, Irgacure 184 (product name) manufactured by BASF), bis(2,4,6-trimethylbenzoyl)-phenylphosphine oxide (for example, Irgacure 819 (product name) manufactured by BASF), 2,4,6-trimethylbenzoyl-diphenyl-phosphinoxide (for example, Irgacure TPO (product name) manufactured by BASF), 2,2,-dimethoxy-1,2-diphenylethan-1-one (for example, Irgacure 651 (product name) manufactured by BASF), 1- [4-(2-hydroxyethoxy)-phenyl] -2-hydroxy-2-methyl-1-propan-1-one, or 2-methyl-1-(4-methylthiophenyl)-2-morpholinopropan-1-one can be preferably used.

In a case of including a photoradical polymerization initiator, a content of the photoradical polymerization initiator in the curable resin composition according to the embodiment of the present invention is preferably 0.01% to 5.0% by mass, more preferably 0.05% to 1.0% by mass, and still more preferably 0.05% to 0.5% by mass.

The curable resin composition according to the embodiment of the present invention may include both the photoradical polymerization initiator and the thermal radical polymerization initiator. In this case, the total content of the photoradical polymerization initiator and the thermal radical polymerization initiator in the above-described curable resin composition is preferably 0.01% to 5% by mass, more preferably 0.05% to 1.0% by mass, and still more preferably 0.05% to 0.5% by mass.

### Other Additives and the Like

The curable resin composition according to the embodiment of the present invention may include additives such as a polymer or a monomer other than the above-described components, a dispersant, a plasticizer, a heat stabilizer, a release agent, or the like as long as the gist of the invention is maintained.

### Properties and the Like of Curable Resin Composition

A viscosity of the curable resin composition according to the embodiment of the present invention is preferably 5000 mPa·s or less, more preferably 3000 mPa·s or less, still more preferably 2500 mPa·s or less, and particularly preferably 2000 mPa·s or less. By setting the viscosity of the curable resin composition within the above-described range, handleability in a case of obtaining (preferably, molding) a cured product can be improved, and a cured product having high quality can be obtained (preferably, formed). The viscosity of the curable resin composition is preferably 50 mPa·s or more, more preferably 100 mPa·s or more, still more preferably 200 mPa·s or more, and particularly preferably 500 mPa·s or more.

### Cured Product

The cured product according to an embodiment of the present invention is obtained from the curable resin composition according to the embodiment of the present invention. The cured product is obtained by polymerizing a polymerizable compound (near-ultraviolet light-absorbing organic compound having a polymerizable group, (meth)acrylate monomer compound, and the like), but the cured product according to the embodiment of the present invention may include an unreacted monomer.

A cured product obtained by curing the curable resin composition according to the embodiment of the present invention is transparent, has a low Abbe number (vd), and has a low refractive index (nF).

For example, in a case where the above-described cured product is formed into a sheet having a thickness of 6 µm, it is possible to obtain a transmittance of 83% or more at a wavelength of 780 nm. The transmittance means a transmittance measured by a spectrophotometer (for example, a spectrophotometer “V-670” manufactured by JASCO Corporation).

In the present invention, the “refractive index (nF)” is a refractive index at a wavelength of 486.13 nm. In addition, the “Abbe number (vd)” is a value calculated from refractive index measurement values at different wavelengths by the following equation.

$$\text{νd} \text{=} \left(\text{nd} \text{-} \text{1}\right) / \left(\text{nF} \text{-} \text{nC}\right)$$

Here, nd represents a refractive index at a wavelength of 587.56 nm, nF represents a refractive index at a wavelength of 486.13 nm, and nC represents a refractive index at a wavelength of 656.27 nm.

The Abbe number vd of the cured product obtained by curing the curable resin composition according to the embodiment of the present invention is not particularly limited, but is preferably 30 or less, more preferably 27 or less, still more preferably 25 or less, and particularly preferably 23 or less. In addition, the Abbe number of the above-described cured product is not particularly limited, but it is preferably 5 or more, more preferably 10 or more, still more preferably 15 or more, and particularly preferably 17 or more. The Abbe number of the above-described cured product is preferably 15 or more and 25 or less.

A refractive index nd (refractive index at a wavelength of 587.56 nm)) of the cured product obtained by curing the curable resin composition according to the embodiment of the present invention is preferably 1.45 or more and 1.60 or less, and more preferably 1.50 or more and 1.55 or less.

A birefringence Δn (in the present invention, sometimes referred to as a birefringence Δn(587 nm)) of the cured product of the curable resin composition according to the embodiment of the present invention at a wavelength of 587 nm is preferably 0.00 ≤ Δn ≤ 0.01. The birefringence Δn(587 nm) is more preferably 0.001 or less and still more preferably less than 0.001. The lower limit value of the birefringence Δn(587 nm) may be 0.00001 or 0.0001.

The birefringence Δn(587 nm) of the cured product can be obtained by the following method. A film-shaped sample is produced, and using a birefringence evaluation device (for example, WPA-100, manufactured by Photonic Lattice, Inc.), a birefringence within a 10 mm diameter circle including the center of the sample is measured. Thereafter, the birefringence Δn(587 nm) can be obtained by obtaining the average value of birefringence at a wavelength of 587 nm.

### Method for Producing Cured Product

The cured product according to the embodiment of the present invention can be produced by a method including at least one of a step of photocuring the curable resin composition according to the embodiment of the present invention or a step of heat-curing the curable resin composition according to the embodiment of the present invention. Among these, a method for producing the cured product preferably includes a step of forming a semi-cured product by irradiating the curable resin composition with light or heating the curable resin composition; and a step of forming a cured product by irradiating the obtained semi-cured product with light or heating the obtained semi-cured product.

As each of the “step of forming a semi-cured product”, the “step of forming a cured product”, and the “semi-cured product”, the description of the “step of forming a semi-cured product”, the “step of forming a cured product”, and the “semi-cured product” in [0106] to [0117], [0118] to [0124], and [0125] of WO2019/044863A can be adopted as they are.

### Use of Curable Resin Composition

The use of the curable resin composition according to the embodiment of the present invention is not particularly limited, but is it preferably used as a material for producing a diffractive optical element. In particular, the resin composition according to the embodiment of the present invention is used as a material for producing a low Abbe number diffractive optical element in a multilayer diffractive optical element, and can provide excellent diffraction efficiency.

### Diffractive Optical Element

A diffractive optical element according to the embodiment of the present invention includes the cured product according to the embodiment of the present invention, in which the diffractive optical element includes a surface having a diffraction grating shape and formed of the cured product.

A diffractive optical element formed by curing the curable resin composition according to the embodiment of the present invention preferably has a maximum thickness of 2 µm to 100 µm. The maximum thickness is more preferably 2 µm to 50 µm and particularly preferably 2 µm to 30 µm. In addition, a level difference of the diffractive optical element is preferably 1 µm to 100 µm and more preferably 1 µm to 50 µm. Furthermore, it is sufficient that a pitch of the diffractive optical element is in a range of 0.1 mm to 10 mm, and it is preferable that the pitch is changed according to the required optical aberration in the same diffractive optical element.

The diffractive optical element can be produced according to, for example, the following procedure.

The curable resin composition is sandwiched between a surface of a mold, which is processed into a diffraction grating shape, and a transparent substrate. Thereafter, the curable resin composition may be pressurized and stretched to a desired range. In the sandwiched state, the curable resin composition is irradiated with light from the transparent substrate side to cure the curable resin composition. Thereafter, the cured product is released from the mold. After the mold release, the cured product may be further irradiated with light from the side opposite to the transparent substrate side.

Examples of the transparent substrate include a flat glass, and a flat transparent resin (such as (meth)acrylic resin, polycarbonate resin, and polyethylene terephthalate).

The transparent substrate used in the above-described production may be included in the diffractive optical element as it is, or may be peeled off.

The surface of the mold, which is processed into a diffraction grating shape, is preferably a chromium nitride-treated surface. As a result, good mold releasability can be obtained, and the producing efficiency of the diffractive optical element can be improved.

Examples of the chromium nitride treatment include a method for forming a chromium nitride film on the mold surface. As the method for forming a chromium nitride film on the mold surface, a chemical vapor deposition (CVD) method and a physical vapor deposition (PVD) method can be exemplified. The CVD method is a method in which a raw material gas including chromium and a raw material gas including nitrogen are reacted at a high temperature to form a chromium nitride film on a surface of a base substance. In addition, the PVD method is a method (arc-type vacuum vapor deposition method) for forming a chromium nitride film on a surface of a base substance using arc discharge. The arc-type vacuum vapor deposition method is a method for forming a film of a compound by reacting ionized metals with a reaction gas on the surface of the base substance. Specifically, a cathode (evaporation source) formed with, for example, chromium in a vacuum container, is disposed, arc discharge occurs between the cathode and a wall surface of the vacuum container through a trigger, ionization of metal by arc plasma is performed at the same time of evaporating the cathode, a negative voltage is applied to the base substance, and a reaction gas (for example, nitrogen gas) is introduced into the vacuum container at approximately several tens mTorr (1.33 Pa).

As the light used for the light irradiation curing the curable resin composition, ultraviolet rays or visible rays are preferable and ultraviolet rays are more preferable. For example, a metal halide lamp, a low pressure mercury lamp, a high pressure mercury lamp, an ultra-high pressure mercury lamp, a germicidal lamp, a xenon lamp, a light emitting diode (LED) light source lamp, and the like are suitably used. The illuminance of ultraviolet light used for the light irradiation curing the curable resin composition is preferably 1 to 100 mW/cm², more preferably 1 to 75 mW/cm², and still more preferably 5 to 50 mW/cm². The curable resin composition may be irradiated with ultraviolet light having different illuminance multiple times. The exposure amount of ultraviolet light is preferably 0.4 to 10 J/cm², more preferably 0.5 to 5 J/cm², and still more preferably 1 to 3 J/cm². The atmosphere during the light irradiation is preferably an atmosphere replaced with air or an inert gas, and more preferably an atmosphere in which air is replaced with nitrogen until the oxygen concentration is 1% or less.

### Multilayer Diffractive Optical Element

The multilayer diffractive optical element according to an embodiment of the present invention includes a first diffractive optical element and a second diffractive optical element, in which the first diffractive optical element is a diffractive optical element formed of the cured product according to the embodiment of the present invention, and the surface of the first diffractive optical element, which has a diffraction grating shape, and a surface of the second diffractive optical element, which has a diffraction grating shape, face each other. It is preferable that the surfaces having the diffraction grating shapes are in contact with each other.

It is preferable that a multilayer diffractive optical element is formed by including, as a first diffractive optical element, the diffractive optical element formed by curing the curable resin composition according to the embodiment of the present invention, and further overlapping a second diffractive optical element formed of a different material such that the first diffractive optical element and the second diffractive optical element face each other in lattice-shaped surfaces. In this case, it is preferable that the lattice-shaped surfaces are in contact with each other.

By forming the second diffractive optical element with a material having a higher refractive index and higher Abbe number than the first diffractive optical element, it is possible to suppress the occurrence of flare, and the like, and sufficiently utilize a chromatic aberration reducing effect of the multilayer diffractive optical element.

An Abbe number vd of the second diffractive optical element is not particularly limited, but is preferably more than 30, more preferably 35 or more, and still more preferably 40 or more. In addition, the Abbe number vd of the second diffractive optical element is not particularly limited, but is preferably 70 or less, more preferably 60 or less, and still more preferably 50 or less. Among these, the Abbe number vd of the second diffractive optical element is preferably 35 to 60.

A refractive index nd of the second diffractive optical element is preferably 1.55 or more and 1.70 or less and more preferably 1.56 or more and 1.65 or less. In addition, the refractive index nd of the second diffractive optical element is set to be larger than the refractive index nd of the first diffractive optical element used simultaneously in the multilayer diffractive optical element.

The material for forming the second diffractive optical element is not particularly limited as long as a cured product having a high refractive index and a high Abbe number is obtained. For example, a curable resin composition including a (meth)acrylate monomer compound having a sulfur atom, a halogen atom, or an aromatic structure, a curable resin composition including zirconium oxide and a (meth)acrylate monomer compound, and the like can be used.

The multilayer diffractive optical element can be produced according to, for example, the following procedure.

A material for forming the second diffractive optical element is sandwiched between a diffraction grating shape surface (surface obtained after the mold release) of a diffractive optical element formed by curing the curable resin composition according to the embodiment of the present invention, and a transparent substrate. Thereafter, the material may be pressurized and stretched to a desired range. In the sandwiched state, the material is irradiated with light from the transparent substrate side to cure the material. Thereafter, the cured product is released from the mold.

That is, as the multilayer diffractive optical element according to the embodiment of the present invention, it is preferable that the first diffractive optical element, the second diffractive optical element, and the transparent substrate are arranged in this order.

Examples of the transparent substrate include the same examples as the transparent substrate used in a case of producing the diffractive optical element (first diffractive optical element).

The transparent substrate used in the above-described production may be included in the multilayer diffractive optical element as it is, or may be peeled off.

The multilayer diffractive optical element preferably has a maximum thickness of 50 µm to 20 mm. The maximum thickness is more preferably 50 µm to 10 mm and particularly preferably 50 µm to 3 mm.

### Lens

The above-described diffractive optical element and multilayer diffractive optical element can be used as a lens, respectively.

A film or a member can be provided on the surface or the periphery of the lens depending on the environment in which the lens is used or the use of the lens. For example, a protective film, an anti-reflection film, a hard coat film, and the like can be formed on the surface of the lens. In addition, the lens can be used as a composite lens in which a glass lens or a plastic lens is laminated on the lens. Furthermore, the periphery of the lens can be fitted into a base material holding frame or the like, and fixed.

However, these films, frames, and the like are members added to the lens, and are distinguished from the lens itself in the present specification.

The lens is preferably used as an image pick-up lens in a mobile phone, a digital camera, and the like, an imaging lens in a television, a video camera, and the like, and an in-vehicle lens.

### EXAMPLES

Hereinafter, the present invention will be described in more detail based on Examples. The materials, amounts used, proportions, treatment details, treatment procedures, and the like described in the following examples can be appropriately modified as long as the gist of the invention is maintained. Therefore, the scope of the present invention should not be construed as being limited to the following specific examples.

### Synthesis Example

A near-ultraviolet light-absorbing organic compound, indium tin oxide particles, and a polymer dispersant were synthesized as follows.

The abbreviations used in the synthesis of each compound described below indicate the following compounds. The room temperature means 25° C.

EDAC: 1-ethyl-3-(3-dimethylaminopropyl)carbodiimide hydrochloride
HPMA: hydroxypropyl methacrylate
DMAc: N,N-dimethylacetamide
THF: tetrahydrofuran
Ac: acetyl group
Et: ethyl group

### 1. Synthesis of Near-Ultraviolet Light-Absorbing Organic Compound

### Synthesis Example 1: Synthesis of Compound (I-32)

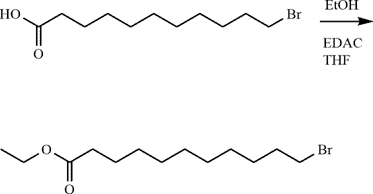

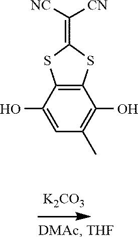

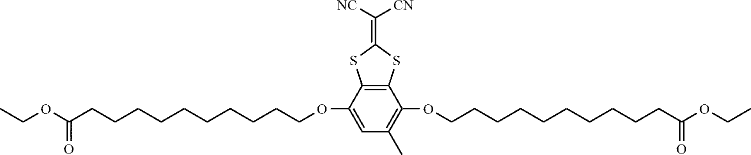

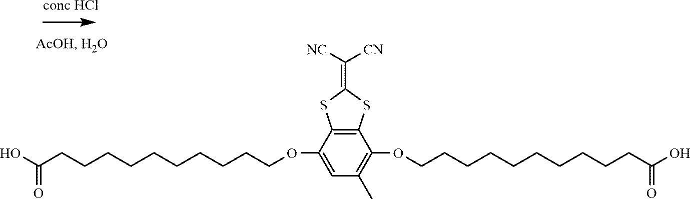

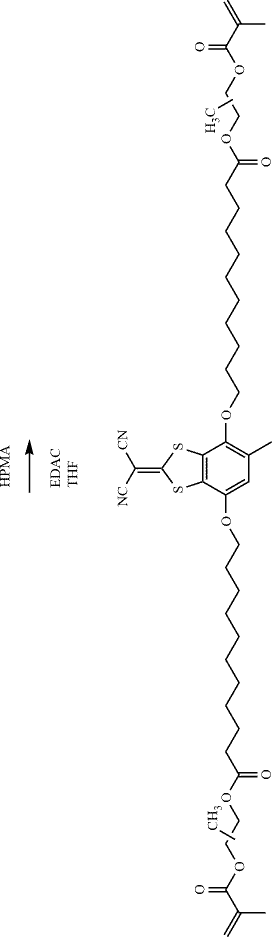

### Synthesis of Compound (I-32A0)

Ethyl 11-bromoundecanoate (compound (I-32A0)) was synthesized by the same method described in Bulletin of the Chemical Society of Japan, 81, 1518 (yield: 90%).

### Synthesis of Compound (I-32A)

A compound (I-1D) was synthesized according to the method described in Journal of Chemical Crystallography (1997); 27 (9); pp. 515 to 526.

While mixing 36.9 g (125.8 mmol) of the compound (I-32A0), 15 g (57.2 mmol) of a compound (I-1D), 17.4 g (125.8 mmol) of potassium carbonate, 60 mL of THF, and 90 mL of N,N-dimethylacetamide, and the mixture was heated so that an internal temperature (liquid temperature) was 80° C. After stirring for 3 hours, 150 mL of ethyl acetate, 180 mL of water, and 30 mL of concentrated hydrochloric acid were added thereto, and the mixture was stirred, washed, and liquid-separated. Next, 150 mL of a 5% sodium hydrogen carbonate aqueous solution was added thereto, and the mixture was stirred, washed, and liquid-separated. Thereafter, 230 mL of methanol was added to the organic layer, and the precipitated crystals were filtered to obtain a compound (I-32A) (yield: 65%).

### Synthesis of Compound (I-32B)

After mixing 20 g (30.6 mmol) of the compound (I-32A), 20 mL of concentrated hydrochloric acid, 240 mL of acetic acid, and 80 mL of water, the mixture was stirred at 80° C. for 1 hour. Thereafter, the temperature was returned to 25° C., 200 mL of water was added thereto, and then the precipitated solid was filtered, washed with methanol and water, and dried at 50° C. to obtain a compound (I-32B) (yield: 90%).

### Synthesis of Compound (I-32)

18 g (28.5 mmol) of the compound (I-32B), 45 mL of THF, 9.1 g (62.8 mmol) of hydroxypropyl methacrylate, 0.4 g (2.9 mmol) of N,N-dimethylaminopyridine, and 12 g (62.8 mmol) of 1-ethyl-3-(3-dimethylaminopropyl)carbodiimide hydrochloride (abbreviation: EDAC) were mixed. After stirring at 40° C. for 2 hours, 300 ml of 1N hydrochloric acid was added thereto, the mixture was washed and liquid-separated, a 5% sodium hydrogen carbonate aqueous solution was added thereto, and the mixture was washed and liquid-separated. An oily composition was obtained by dehydration with magnesium sulfate, filtration, concentration, and then purified by column chromatography to obtain a compound (I-32) (yield: 70%).

¹H-NMR (300 MHz, CDCl₃): δ (ppm) 1.25 to 1.50 (m, 30H), 1.50 to 1.70 (m, 8H), 1.95 (s, 6H), 2.20 to 2.40 (m, 7H), 3.85 (t, 2H), 4.0 (t, 2H), 4.10 to 4.30 (m, 4H), 5.10 to 5.30 (m, 2H), 5.60 (s, 2H), 6.10 (s, 2H), 6.70 (s, 1H)

### <Synthesis Example 2: Synthesis of Compound (I-31)>

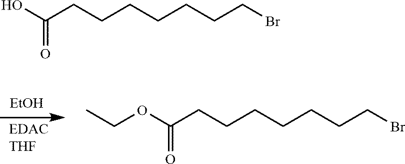

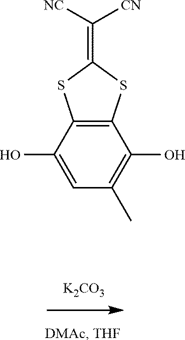

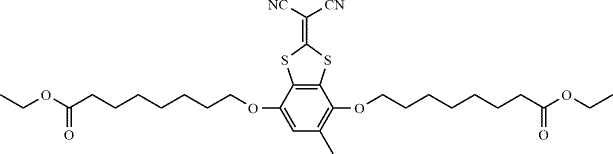

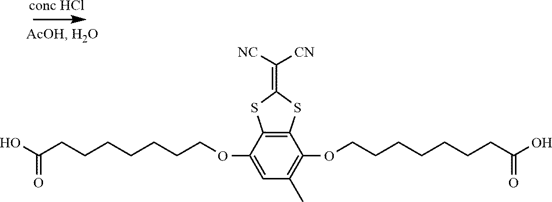

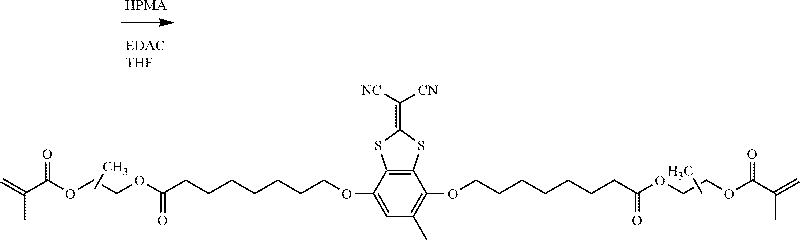

### Synthesis of Compound (I-31A0)

Ethyl 8-bromooctanoate (compound (I-31A0)) was synthesized by the same method as in the synthesis of the compound (I-32A0), except that 11-bromoundecanoic acid was changed to 8-bromooctanoic acid (yield: 88%).

### Synthesis of Compound (I-31A)

A compound (I-31A) was synthesized in the same method as in the synthesis of the compound (I-32A), except that the compound (I-32A0) was changed to the compound (I-31A0) (yield: 67%).

### Synthesis of Compound (I-31B)

A compound (I-31B) was synthesized in the same method as in the synthesis of the compound (I-32B), except that the compound (I-32A) was changed to the compound (I-31A) (yield: 97%).

### Synthesis of Compound (I-31)

A compound (I-31) was synthesized in the same method as in the synthesis of the compound (I-32), except that the compound (I-32B) was changed to the compound (I-31B) (yield: 60%).

¹H-NMR (300 MHz, CDCl₃): δ (ppm) 1.25 to 1.50 (m, 18H), 1.50 to 1.70 (m, 4H), 1.50 to 1.70 (quint, 4H), 1.95 (s, 6H), 2.20 to 2.40 (m, 7H), 3.85 (t, 2H), 4.0 (t, 2H), 4.10 to 4.30 (m, 4H), 5.10 to 5.30 (m, 2H), 5.60 (s, 2H), 6.10 (s, 2H), 6.70 (s, 1H)

### <Synthesis Example 3: Synthesis of Compound (I-33)>

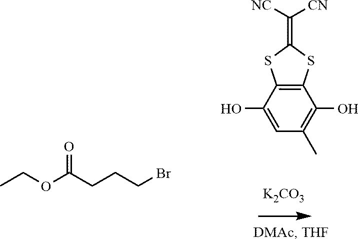

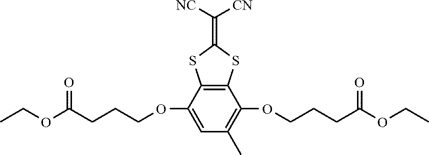

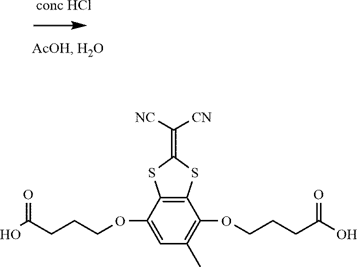

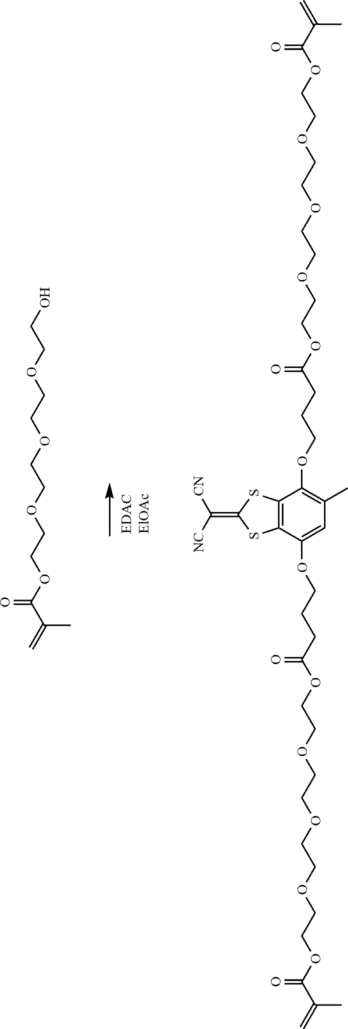

### Synthesis of Compound (I-33A)

A compound (I-33A) was synthesized in the same method as in the synthesis of the compound (I-32A), except that the compound (I-32A0) was changed to ethyl bromobutyrate (manufactured by Wako Pure Chemical Corporation) (yield: 62%).

### Synthesis of Compound (I-33B)

A compound (I-33B) was synthesized in the same method as in the synthesis of the compound (I-32B), except that the compound (I-32A) was changed to the compound (I-33A) (yield: 98%).

### Synthesis of Compound (1-33)

12.4 g (28.5 mmol) of the compound (I-33B), 45 mL of ethyl acetate, 16.5 g (62.8 mmol) of Blemmer PE-200 (product name, manufactured by NOF Corporation, hydroxyl-terminated polyalkylene glycol monomethacrylate), 0.4 g (2.9 mmol) of N,N-dimethylaminopyridine, and 12 g (62.8 mmol) of 1-ethyl-3-(3-dimethylaminopropyl)carbodiimide hydrochloride (abbreviation: EDAC) were mixed. After stirring at 40° C. for 2 hours, 300 ml of 1N hydrochloric acid was added thereto, the mixture was washed and liquid-separated, a 5% sodium hydrogen carbonate aqueous solution was added thereto, and the mixture was washed and liquid-separated. An oily composition was obtained by dehydration with magnesium sulfate, filtration, concentration, and then purified by column chromatography to obtain a compound (I-33) (yield: 48%).

¹H-NMR (300 MHz, CDCl₃): δ (ppm) 1.93 (s, 6H), 2.10 to 2.20 (m, 4H), 2.32 (s, 3H), 2.50 to 2.70 (m, 4H), 3.60 to 3.90 (m, 24H), 4.10 to 4.30 (m, 12H), 5.60 (s, 2H), 6.10 (s, 2H), 6.70 (s, 1H)

### Synthesis Example 4: Synthesis of Compound (I-26)

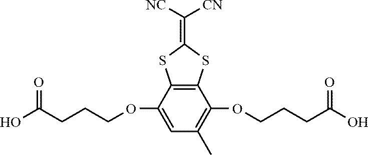

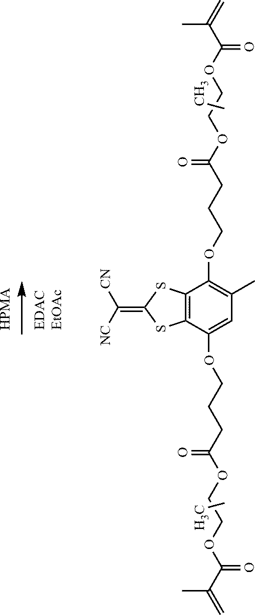

A compound (I-26) was synthesized in the same method as in the synthesis of the compound (I-32), except that the compound (I-32B) was changed to the compound (I-33B) (yield: 57%).

¹H-NMR (400 MHz, CDCl₃): δ (ppm) 1.20 to 1.35 (m, 6H), 1.93 (s, 6H), 2.10 to 2.20 (m, 4H), 2.32 (s, 3H), 2.60 to 2.75 (m, 4H), 3.92 (t, 2H), 4.10 to 4.30 (m, 6H), 5.15 to 5.35 (m, 2H), 5.57 (s, 2H), 6.10 (s, 2H), 6.69 (s, 1H)

### Synthesis Example 5: Synthesis of Compound (A-35)

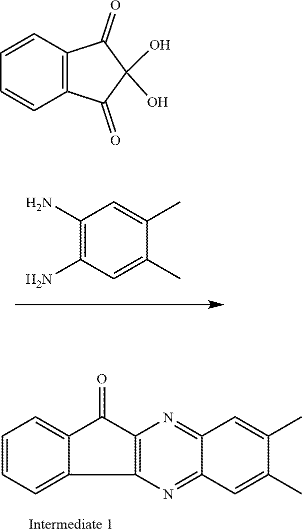

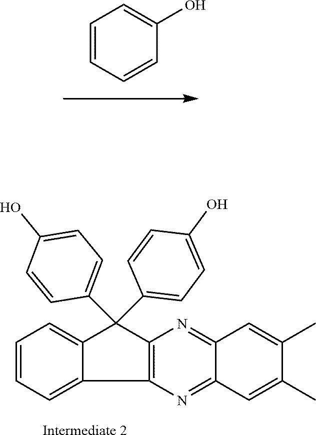

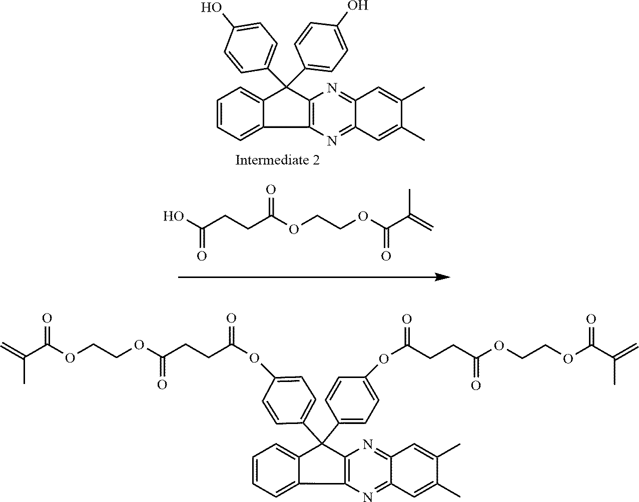

### Synthesis of Intermediate 1

50 mL of ethanol and 10 mL of acetic acid were added to 25.6 g of 4,5-dimethyl-1,2-phenylenediamine and 35.6 g of ninhydrin, and the mixture was reacted at 70° C. for 3 hours. The reaction solution was cooled to room temperature, and the precipitated crystals were collected by filtration, washed with ethanol, and dried to obtain 41.1 g of an intermediate 1.

¹H-NMR (300 MHz, CDCl₃): δ 2.49 ppm (s, 3H), 2.51 ppm (s, 3H), 7.52 to 7.58 ppm (t, 1H), 7.71 to 7.76 ppm (t, 1H), 7.85 to 7.95 ppm (m, 3H), 8.02 to 8.08 ppm (d, 1H)

### Synthesis of Intermediate 2

22 g of the intermediate 1 and 32 g of phenol were dissolved in 20 mL of methanesulfonic acid and 20 mL of acetonitrile. The reaction solution was heated, and 0.3 mL of 3-mercaptopropionic acid was added dropwise thereto while maintaining the temperature at 90° C. After stirring for 3 hours, 200 mL of acetonitrile and 100 mL of water were added thereto, and the reaction solution was stirred in an ice bath for 2 hours. The precipitated crystals were collected by filtration, washed with methanol, and dried to obtain 26 g of an intermediate 2.

¹H-NMR (300 MHz, DMSO-d₆): δ 2.47 ppm (s, 3H), 2.49 ppm (s, 3H), 6.61 to 6.67 ppm (d, 4H), 6.95 to 7.01 ppm (d, 4H), 7.52 to 7.62 ppm (m, 3H), 7.84 ppm (s, 1H), 7.93 ppm (s, 1H), 8.12 to 8.14 ppm (d, 1H), 9.40 ppm (bs, 2H)

### Synthesis of Compound (A-35)

Into a 200 mL three-neck flask, 4.8 g of the intermediate 2, 6.5 g of mono(2-methacryloyloxyethyl) succinate, 140 mg of N,N-dimethylaminopyridine (DMAP), and 50 mL of dichloromethane were charged, and the mixture was stirred in an ice bath for 10 minutes. 5.8 g of 1-ethyl-3-(3-dimethylaminopropyl)carbodiimide hydrochloride (EDAC) was added thereto, and the mixture was reacted at room temperature for 4 hours. The reaction solution was diluted with ethyl acetate, and washed with water, saturated sodium hydrogen carbonate aqueous solution, and saturated saline in this order, and the organic layer was dried with magnesium sulfate. After removing the magnesium sulfate by filtration, the obtained product was purified by silica gel column chromatography using ethyl acetate/hexane as a developing solvent to obtain 7.5 g of a compound (A-35). ¹H-NMR data of the compound (A-35) was as follows.

¹H-NMR (300 MHz, DMSO-d₆): δ 1.80 ppm (s, 6H), 2.47 ppm (s, 3H), 2.49 ppm (s, 3H), 2.62 to 2.72 ppm (m, 4H), 2.80 to 2.90 ppm (m, 4H), 4.25 to 4.35 ppm (m, 8H), 5.58 ppm (s, 2H), 5.97 ppm (s, 2H), 7.00 to 7.10 ppm (d, 4H), 7.20 to 7.30 ppm (d, 4H), 7.55 to 7.70 ppm (m, 3H), 7.84 ppm (s, 1H), 7.93 ppm (s, 1H), 8.16 to 8.22 ppm (d, 1H)

### Synthesis Example 6: Synthesis of Compound (VII-1)

The following compound (VII-1) was synthesized in the same manner as in Example 2 of JP2014-43565A.

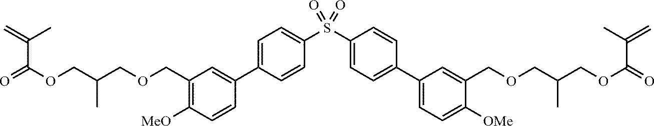 [SMILES: C=C(C)C(=O)OCC(C)COCc1cc(-c2ccc(S(=O)(=O)c3ccc(-c4ccc(OC)c(COCC(C)COC(=O)C(=C)C)c4)cc3)cc2)ccc1OC]

Absorption spectra (absorbance) of the near-ultraviolet light-absorbing organic compounds produced above were measured by the following procedure.

Each compound was precisely weighed in an amount of 50 mg, diluted with tetrahydrofuran (THF) using a 5 mL volumetric flask, and further diluted with THF so that the solution concentration was 1/500 times to prepare a measurement solution. The measurement was performed using UV-2550 (product name) manufactured by Shimadzu Corporation.

First, a square quartz cell (cell length: 10 mm) containing a control sample (THF) in both the sample optical path and the control optical path was placed, and the absorbance in a wavelength region of 250 to 800 nm was adjusted to zero. Next, the sample in the sample optical path-side cell was replaced with the measurement solution of the near-ultraviolet light-absorbing organic compound prepared above, and the absorption spectrum at 250 to 800 nm was measured. None of the compounds exhibited substantially light absorption at a wavelength of 410 to 800 nm.

Among maximal values in a range of 300 to 400 nm obtained from the measurement results, a wavelength λmax with the highest absorbance, a maximum absorbance A_{λmax} in 300 to 400 nm, an absorbance A₄₁₀ at a wavelength 410 nm, an absorbance A₄₃₀ at a wavelength 430 nm, and values calculated from the following expressions are shown in Table 1.

TABLE 1

| Compound | I-26 | I-31 | I-32 | I-33 | A-35 | VII-1 |
|---|---|---|---|---|---|---|
| λmax [nm] | 374 | 374 | 374 | 374 | 374 | 307 |
| A_{λmax} | 0.693 | 0.605 | 0.544 | 0.621 | 0.640 | 1.019 |
| A₄₁₀ | 0.004 | 0.004 | 0.004 | 0.004 | 0.000 | 0.000 |
| A₄₃₀ | 0.000 | 0.000 | 0.000 | 0.000 | 0.000 | 0.000 |
| PA-I | 0.994 | 0.993 | 0.993 | 0.994 | 1.000 | 1.000 |
| PA-II | 0.994 | 0.993 | 0.993 | 0.994 | 1.000 | 1.000 |
| PA-III | 0.0191 | 0.0167 | 0.0150 | 0.0171 | 0.0177 | 0.0099 |

In the above table, PA-I, PA-II, and PA-III are values calculated as follows.

$$\text{PA-I} \text{=} \left({\text{A}}_{λ max} \text{-} {\text{A}}_{410}\right) / {\text{A}}_{λ max}$$

$$\text{PA-II} \text{=} \left({\text{A}}_{λ max} \text{-} {\text{A}}_{410}\right) / \left({\text{A}}_{λ max} \text{-} {\text{A}}_{430}\right)$$

$$\text{PA-III} \text{=} \left({\text{A}}_{λ max} \text{-} {\text{A}}_{410}\right) / \left(410 \text{-} λ max\right)$$

### 2. Synthesis of ITO Particles (ITO-1)

75 ml of oleic acid (manufactured by Sigma-Aldrich, Inc., technical grade, 90%), 10.060 g (34.5 mmol) of indium acetate (manufactured by Alfa Aesar, 99.99%), and 1.079 g (3.0 mmol) of tin (IV) acetate (manufactured by Alfa Aesar) were added in a flask. The mixture in the flask was heated at 160° C. for 1 hour under an environment of nitrogen flow to obtain a yellow transparent precursor solution.

Subsequently, 90 ml of oleyl alcohol (manufactured by FUJIFILM Wako Pure Chemical Corporation (formerly manufactured by WAKO CHEMICAL CO., LTD.), standard content: 65% or more) was charged into another flask, and heated at 290° C. in a nitrogen flow. Using a syringe pump, the precursor solution was added dropwise to the heated oleyl alcohol at a rate of 1.75 mL/min. After the completion of the dropwise addition of the precursor solution, the obtained reaction solution was retained at 290° C. for 120 minutes, and thereafter, the heating was stopped and the reaction solution was cooled to room temperature.

After adding ethanol to the obtained reaction solution, centrifugation was performed to precipitate particles. The removal of the supernatant and the redispersion of the particles in toluene were repeated 3 times to obtain a toluene dispersion liquid (ITO solid content: 4.75% by mass, surface treatment surface-modified component solid content: 0.25% by mass, total solid concentration in dispersion liquid: 5.00% by mass) of ITO particles (ITO-1) coordinated with oleic acid.

In a case where the above-descried ITO particles (ITO-1) were observed by TEM (product name: JFM-ARM300F2 GRAND, manufactured by JEOL Ltd.), the average primary particle diameter was 28.5 nm. Specifically, the measurement was performed based on the above-described method for measuring the average primary particle diameter of ITO particles.

### 3. Synthesis of Polymer Dispersant

### Synthesis of Polymer Dispersant (P-1)

24.0 g of benzyl methacrylate (manufactured by Wako Pure Chemical Corporation) and 1.80 g of mercaptosuccinic acid (manufactured by Wako Pure Chemical Corporation) were dissolved in 28 mL of methyl ethyl ketone and heated to 70° C. under a nitrogen stream. A solution in which 0.24 g of a polymerization initiator (manufactured by Wako Pure Chemical Corporation, product name: V-65) was dissolved in 12 mL of methyl ethyl ketone was added dropwise to this solution over 30 minutes. After the completion of the dropwise addition, the reaction was further performed at 70° C. for 4.5 hours. After allowing to cool, the reaction solution was added dropwise to a cooled mixed solution of 200 mL of water and 600 mL of methanol, and the precipitated powdery substance was collected by filtration and dried to obtain 15 g of a polymer dispersant (P-1) having a carboxy group as an acidic group at one terminal. The polymer dispersant (P-1) was substantially composed of a polymer having a carboxy group at one terminal.

The weight-average molecular weight of the obtained polymer was 8000 in terms of standard polystyrene according to a gel permeation chromatography (GPC) method measured under the following measurement conditions, and the dispersity (Mw/Mn; Mn: number-average molecular weight) was 1.90. In addition, in a case where the number in mg of potassium hydroxide required to neutralize free fatty acid present in 1 g of the obtained polymer was measured to obtain an acid value, the acid value was 28 mgKOH/g.

### Measurement Condition

Measuring instrument: HLC-8320GPC (product name, manufactured by Tosoh Corporation)

Column: connection of TOSOH TSKgel Super HZM-H (product name, manufactured by Tosoh Corporation), TOSOH TSKgel Super HZ4000 (product name, manufactured by Tosoh Corporation), and TOSOH TSKgel Super HZ2000 (product name, manufactured by Tosoh Corporation)
Carrier: THF
Measurement temperature: 40° C.
Carrier flow rate: 0.35 mL/min
Sample concentration: 0.1%
Detector: refractive index (RI) detector

### Synthesis of Polymer Dispersants (P-2) to (P-13)

Polymer dispersants (P-2) to (P-13) having an acidic group at one terminal of the polymer chain were synthesized in the same manner as the polymer dispersant (P-1), except that, in the above-described synthesis of the polymer dispersant (P-1), instead of benzyl methacrylate, the (meth)acrylate monomers described in the columns of constituent monomers 1 and 2 in Table 2 below were used so that the acid value and weight-average molecular weight (Mw) were adjusted as described in Table 2 below.

In the synthesis of the polymer dispersant (P-8), mercaptoethanol was used instead of mercaptosuccinic acid to produce a polymer having a hydroxyl group at one terminal of the polymer. Further, by reacting the hydroxyl group with pyrophosphoric acid, a polymer having a phosphonooxy group at one terminal of the polymer was produced.

### Example

### 1. Preparation of Curable Resin Composition

0.43 g of the compound (I-32), 0.07 g of the polymer dispersant (P-1), and 0.15 g of 2-ethylhexyl methacrylate (2-EHMA, manufactured by Tokyo Chemical Industry Co., Ltd.) were added and dissolved in 7.0 g (solid content: 0.35 g) of a toluene dispersion liquid of ITO-1 prepared above. Toluene was distilled off by suction under reduced pressure while heating in a water bath at approximately 70° C. After the distillation, 0.002 g of IRGACURE 819 (product name, manufactured by BASF) having the following structure was added to the obtained mixture and dissolved, thereby preparing a curable resin composition No. 101.

Same as the preparation of the curable resin composition 101, curable resin compositions Nos. 102 to 119, c01 to c05, r01, and r02 were prepared so as to have compositional ratios shown in the tables below.

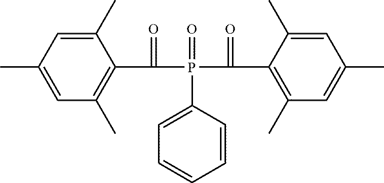

### Evaluation 1: Appearance of Composition

The curable resin composition prepared above was visually observed, and appearance of the composition was evaluated according to the following standard. In this test, “A” or higher is an acceptable level.
- Evaluation standard -
A: curable resin composition was uniform and transparent.
B: curable resin composition was uniform, but was opaque due to fine aggregation.
C: curable resin composition was non-uniform, and the presence of aggregates in the curable resin composition could be visually confirmed.

### 2. Production of Cured Product

The curable resin composition prepared above was sandwiched between hydrophobically treated glass plates, irradiated with UV under the conditions of integrated light intensity of 1.0 J/cm² and illuminance of 30 mW/cm² using a UV irradiation device (EXECURE 3000 (product name), manufactured by HOYA CANDEO OPTRONICS CORPORATION), and irradiated with UV under the conditions of integrated light intensity of 1.0 J/cm² and illuminance of 5 mW/cm² to produce a cured product. The film thickness of the cured product obtained as described above was 6 µm.

### Optical Characteristics Measurement

### Measurement of Refractive Index and Vd

Using the cured product produced under the above-described conditions, refractive index at wavelengths of 587.56 nm, 486.13 nm, and 656.27 nm was measured with a multi-wavelength Abbe refractometer DR-M2 (product name, manufactured by ATAGO CO., LTD.), and an Abbe number vd was calculated by the following expression.

$$\text{vd=(nd-1)/(nF-nC)}$$

Here, nd represents a refractive index at a wavelength of 587.56 nm, nF represents a refractive index at a wavelength of 486.13 nm, and nC represents a refractive index at a wavelength of 656.27 nm.

All of the cured products produced under the above-described conditions had a refractive index nd of 1.50 to 1.56 at the wavelength of 587.56 nm.

The calculated Abbe number vd was evaluated according to the following standard. In this test, “A” or higher is an acceptable level.
- Evaluation standard -
A: 18 or more and less than 21
B: 21 or more and less than 24
C: 24 or more

### Measurement of Transmittance

With regard to the cured product produced under the above-described conditions, using a spectrophotometer UV-2600 (product name, manufactured by Shimadzu Corporation), a transmittance at a wavelength of 400 to 800 nm was measured, and a transmittance at 780 nm was evaluated according to the following standard. In this test, “A” or higher is an acceptable level.
- Evaluation standard -
A: transmittance was 86% or more.
B: transmittance was 83% or more and less than 86%.
C: transmittance was less than 83%.

### Evaluation 2: Long-Term Dispersion Stability

The curable resin composition prepared in [1. Preparation of curable resin composition] described above was stored in a vial and allowed to stand at a condition of 25° C. The appearance of the composition was visually observed every week, and the composition was applied onto a slide glass and observed at 100x magnification (eyepiece 10x, objective 10x) for aggregation using a polarization microscope (ECLIPSE LV100 POL (product name), manufactured by Nikon Corporation), thereby observing whether aggregate occurred. It was determined that aggregate occurred in a case where aggregation was confirmed by at least one of visual observation or observation with a polarization microscope, and long-term dispersion stability of the curable resin composition was evaluated according to the following standard. In this test, “C” or higher is an acceptable level, and “B” or higher is preferable.
- Evaluation standard -
A: composition was stable even after storage for 3 months, and no aggregation was observed.
B: aggregate occurred in 1 month or more and less than 3 months.
C: aggregate occurred in 2 weeks or more and less than 1 month.
D: aggregate occurred in less than 2 weeks.

| | | | | | | | | | |
|---|---|---|---|---|---|---|---|---|---|
| Curable resin composition | | 101 | 102 | 103 | 104 | 105 | 106 | 107 | 108 |
| ITO particles: ITO-1 | Blending amount (part by mass) | 35 | 35 | 35 | 35 | 35 | 35 | 35 | 35 |
| Polymer dispersant | Type | P-1 | P-1 | P-2 | P-3 | P-4 | P-5 | P-1 | P-1 |
| | Constituent monomer 1 | BnMA | BnMA | BnMA | BnMA | PhMA | PEMA | BnMA | BnMA |
| | Constituent monomer 2 | – | – | – | – | – | – | – | – |
| | Acidic group (adsorptive group) | Carboxy group | | | | | | | |
| | Acid value | 28 | 28 | 41 | 15 | 25 | 31 | 28 | 28 |
| | Mw | 8000 | 8000 | 5000 | 13000 | 9000 | 6000 | 8000 | 8000 |
| | Content of General Formula (P)(mol%) | 100 | | | | | | | |
| | Blending amount (part by mass) | 7 | 7 | 7 | 7 | 7 | 7 | 7 | 7 |
| Blending amount of polymer dispersant with respect to 100 parts by mass of blending amount of ITO particles (part by mass) | | 20 | 20 | 20 | 20 | 20 | 20 | 20 | 20 |
| Near-ultraviolet light-absorbing organic compound | Type | I-32 | I-32 | I-32 | I-32 | I-32 | I-32 | I-26 | I-31 |
| | Blending amount (part by mass) | 43 | 57 | 43 | 43 | 43 | 43 | 31 | 43 |
| | Maximal wavelength | A | A | A | A | A | A | A | A |
| (Meth)acrylate monomer | Type | 2-EHMA | – | 2-EHMA | 2-EHMA | 2-EHMA | 2-EHMA | 2-EHMA | 2-EHMA |
| | Blending amount (part by mass) | 15 | – | 15 | 15 | 15 | 15 | 15 | 15 |
| Photopolymerization initiator: IRGACURE 819 | Blending amount (part by mass) | 0.2 | 0.2 | 0.2 | 0.2 | 0.2 | 0.2 | 0.2 | 0.2 |
| Resin cured product | vd | A | A | A | A | A | A | A | A |
| | Transmittance (780 nm) | A | A | A | A | A | A | A | A |
| Appearance of composition | | A | A | A | A | A | A | A | A |
| Long-term dispersion stability | | A | A | A | A | A | A | A | A |

| | | | | | | | | | |
|---|---|---|---|---|---|---|---|---|---|
| Curable resin composition | | 109 | 110 | 111 | 112 | 113 | 114 | 115 | 116 |
| ITO particles: ITO-1 | Blending amount (part by mass) | 35 | 35 | 35 | 35 | 35 | 35 | 35 | 35 |
| Polymer dispersant | Type | P-1 | P-6 | P-7 | P-8 | P-9 | P-10 | P-1 | P-1 |
| | Constituent monomer 1 | BnMA | BnMA | BnMA | BnMA | BnMA | BnMA | BnMA | BnMA |
| | Constituent monomer 2 | – | tBuMA | tBuMA | – | – | tBuMA | – | – |
| | Acidic group (adsorptive group) | Carboxy group | | | Phosphonooxy group | Carboxy group | | | |
| | Acid value | 28 | 29 | 30 | 28 | 15 | 28 | 28 | 28 |
| | Mw | 8000 | 7000 | 6000 | 8000 | 28000 | 8000 | 8000 | 8000 |
| | Content of General Formula (P) (mol%) | 100 | 50 | 15 | 100 | | 5 | 100 | |
| | Blending amount (part by mass) | 7 | 7 | 7 | 7 | 7 | 7 | 7 | 7 |
| Blending amount of polymer dispersant with respect to 100 parts by mass of blending amount of ITO particles (part by mass) | | 20 | 20 | 20 | 20 | 20 | 20 | 20 | 20 |
| Near-ultraviolet light-absorbing organic compound | Type | I-33 | I-32 | I-32 | I-32 | I-32 | I-32 | A-35 | VII-1 |
| | Blending amount (part by mass) | 43 | 43 | 43 | 43 | 43 | 43 | 43 | 43 |
| | Maximal wavelength | A | A | A | A | A | A | A | C |
| (Meth)acrylate monomer | Type | 2-EHMA | 2-EHMA | 2-EHMA | 2-EHMA | 2-EHMA | 2-EHMA | 2-EHMA | 2-EHMA |
| | Blending amount (part by mass) | 15 | 15 | 15 | 15 | 15 | 15 | 15 | 15 |
| Photopolymerization initiator: IRGACURE 819 | Blending amount (part by mass) | 0.2 | 0.2 | 0.2 | 0.2 | 0.2 | 0.2 | 0.2 | 0.2 |
| Resin cured product | vd | A | A | A | A | A | A | A | B |
| | Transmittance (780 nm) | A | A | A | A | A | A | A | A |
| Appearance of composition | | A | A | A | A | A | A | A | A |
| Long-term dispersion stability | | A | A | A | C | B | B | A | A |

| | | | | |
|---|---|---|---|---|
| Curable resin composition | | 117 | 118 | 119 |
| ITO particles: ITO-1 | Blending amount (part by mass) | 35 | 35 | 35 |
| Polymer dispersant | Type | P-6 | P-6 | P-6 |
| | Constituent monomer 1 | BnMA | BnMA | BnMA |
| | Constituent monomer 2 | tBuMA | tBuMA | tBuMA |
| | Acidic group (adsorptive group) | Carboxy group | | |
| | Acid value | 29 | 29 | 29 |
| | Mw | 7000 | 7000 | 7000 |
| | Content of General Formula (P) (mol%) | 50 | 50 | 50 |
| | Blending amount (part by mass) | 7 | 7 | 7 |
| Blending amount of polymer dispersant with respect to 100 parts by mass of blending amount of ITO particles (part by mass) | | 20 | 20 | 20 |
| Near-ultraviolet light-absorbing organic compound | Type | I-32 | I-32 | 1-32 |
| | Blending amount (part by mass) | 43 | 43 | 43 |
| | Maximal wavelength | A | A | A |
| (Meth)acylate monomer | Type | DDMA | HDDMA | TMPTMA |
| | Blending amount (part by mass) | 15 | 15 | 15 |
| Photopolymerization initiator: IRGACURE 819 | Blending amount (part by mass) | 0.2 | 0.2 | 0.2 |
| Resin cured product | vd | A | A | A |
| | Transmittance (780 nm) | A | A | A |
| Appearance of composition | | A | A | A |
| Long-term dispersion stability | | A | A | A |

| | | | | | | | | |
|---|---|---|---|---|---|---|---|---|
| Curable resin composition | | c01 | c02 | c03 | c04 | c05 | r01 | r02 |
| ITO particles: ITO-1 | Blending amount (part by mass) | 35 | 35 | 35 | 28 | 35 | 28 | 28 |
| Polymer dispersant | Type | P-11 | P-11 | P-12 | P-1 | P- | | P-13 |
| | Constituent monomer 1 | tBuMA | tBuMA | CyMA | BnMA | BnMA | – | MMA |
| | Constituent monomer 2 | – | – | – | – | – | – | – |
| | Acidic group (adsorptive group) | Carboxy group | | | | | Phosphonooxy group | Carboxy group |
| | Acid value | 30 | 30 | 27 | 28 | 23 | 240 | 24 |
| | Mw | 5000 | 5000 | 6000 | 8000 | 8000 | 460 | 6000 |
| | Content of General Formula (P) (mol%) | 0 | 0 | 0 | 100 | 100 | 0 | 0 |
| | Blending amount (part by mass) | 7 | 7 | 7 | 2.8 | 7 | 2.8 | 2.8 |
| Blending amount of polymer dispersant with respect to 100 parts by mass of blending amount of ITO particles (part by mass) | | 20 | 20 | 20 | 10 | 20 | 10 | 10 |
| Near-ultraviolet light-absorbing organic compound | Type | I-32 | I-32 | I-32 | – | – | I-26 | I-26 |
| | Blending amount (part by mass) | 35 | 43 | 35 | – | – | 31 | 31 |
| | Maximal wavelength | A | A | A | – | – | A | A |
| (Meth)acrylate monomer | Type | 2-EHMA | 2-EHMA | 2-EHMA | HDDA | HDDA | HDDMA | 2-EHMA |
| | Blending amount (part by mass) | 23 | 15 | 23 | 69.2 | 38.2 | 38.2 | 38.2 |
| Photepolymerization initiator: IRGACURE 819 | Blending amount (part by mass) | 0.2 | 0.2 | 0.2 | 0.2 | 0.2 | 0.2 | 0.2 |
| Resin cured product | vd | A | A | A | C | B | A | A |
| | Transmittance (780 nm) | A | A | A | A | B | A | A |
| Appearance of composition | | B | C | B | A | A | A | A |
| Long-term dispersion stability | | – | – | – | A | A | D | D |

### Note to Table

Each component in the tables is as follows.

The description of “-” in each component means that the corresponding component is not contained. In addition, the blending ratio of each component is based on mass, and the blending amount of ITO particles means the amount of solid content in the ITO particle dispersion liquid.

### Polymer Dispersant

P-1 to P-13: polymer dispersants (P-1) to (P-13) produced above

Phosmer PP: product name, manufactured by Unichemical Co., Ltd.

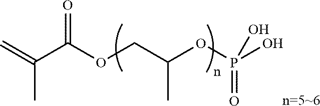

BnMA: benzyl methacrylate
PhMA: phenyl methacrylate
PEMA: phenoxyethyl methacrylate
tBuMA: t-butyl methacrylate
CyMA: cyclohexyl methacrylate
MMA: methyl methacrylate

Among the above-described polymer dispersants, with regard to the polymer dispersant having a carboxy group and the polymer dispersant (P-8) having a phosphonooxy group, each acidic group (adsorptive group) is introduced at one terminal of the polymer as the following structural portion.

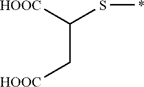 [SMILES: *SC(CC(=O)O)C(=O)O]

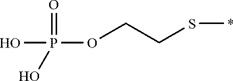 [SMILES: *SCCOP(=O)(O)O]

The unit of the acid value is mgKOH/g.

The weight-average molecular weight (Mw) is a value obtained by rounding off the hundreds digit. The value of Phosmer PP described in the column of Mw is a value described in the sales company catalog.

The content of General Formula (P) means a proportion of the constitutional unit represented by General Formula (P) to all constitutional units constituting the polymer. In Nos. 101 to 119, the content of General Formula (P) corresponds to the proportion of the constituent monomer 1 to the total monomers.

### Near-Ultraviolet Light-Absorbing Organic Compound

I-26, I-31 to I-33, A-35, VII-1: near-ultraviolet light-absorbing organic compounds I-26, I-31 to I-33, A-35, and VII-1 synthesized above

Maximal wavelength: among maximal values of the absorbance in the range of 300 to 400 nm, the wavelength λmax with the highest absorbance was designated as “A” for 380 nm or more, “B” for 340 nm or more and less than 380 nm, and “C” for less than 340 nm.

((Meth)acrylate monomer)
2-EHMA: 2-ethylhexyl methacrylate
DDMA: dodecyl methacrylate
TMOTMA: trimethylolpropane trimethacrylate
HDDA: 1,6-hexanediol diacrylate
HDDMA: 1,6-hexanediol dimethacrylate

“-” in the column of long-term dispersion stability of the curable resin compositions Nos. c01 to c03 means that the composition was not dispersed at the stage of preparation, and the long-term dispersion stability was not evaluated.

From the results shown in Table 2, the following is found.

The cured product obtained from the comparative curable resin composition No. c04 or c05 was unable to achieve both the low Abbe number and the high transmittance in the near-infrared wavelength region. It could be seen that there was a problem in the conventional technique of adjusting the wavelength dependence of the refractive index by adding the ITO particles.

In contrast to these, the cured product obtained from the curable resin composition No. r01 or r02 of Reference Example, containing the ITO particles and the specific near-ultraviolet light-absorbing compound, was able to realize both the low Abbe number and the high transmittance in the near-infrared wavelength region. However, these curable resin compositions were inferior in dispersion stability over a long period of time. In addition, in the comparative curable resin compositions Nos. c01 to c03, which did not use the polymer defined in the present invention as the polymer dispersant, a curable resin composition excellent in dispersion stability of each component could not be obtained.

On the other hand, in the curable resin compositions Nos. 101 to 119 according to the embodiment of the present invention, containing the polymer dispersant defined in the present invention, the obtained cured product realized a low Abbe number and a high transmittance in the near-infrared wavelength region, and the curable resin composition exhibited excellent dispersion stability over a long period of time.

The present invention has been described with the embodiments thereof, any details of the description of the present invention are not limited unless described otherwise, and it is obvious that the present invention is widely construed without departing from the gist and scope of the present invention described in the accompanying claims.

What is claimed is:

1. A curable resin composition comprising:
a near-ultraviolet light-absorbing organic compound;
indium tin oxide particles; and
a polymer having a constitutional unit represented by General Formula (P) and having an acidic group at one terminal, 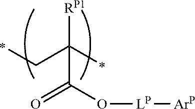
in the formula, L^{p} represents a single bond or a divalent linking group, Ar^{P} represents an aryl group, R^{P1} represents a hydrogen atom or a methyl group, where Ar^{P} does not include the acidic group, and * represents a bonding portion,
wherein, in the near-ultraviolet light-absorbing organic compound, in a case where an absorbance is measured from a wavelength of 800 nm toward a short wavelength side, a wavelength at which a maximal value is first exhibited is present at 300 to 400 nm, and
in a case where an absorbance at a wavelength of λ, nm is defined as A_{λ}, relationships of Expression I to III are satisfied,
$$\left({\text{A}}_{\text{λ} max} {\text{-A}}_{410}\right) / {\text{A}}_{\text{λ} max} \geq 0.97$$
$$1.00 \geq \left({\text{A}}_{\text{λ} max} {\text{-A}}_{410}\right) / \left({\text{A}}_{\text{λ} max} {\text{-A}}_{430}\right) \geq 0.97$$
$$\left({\text{A}}_{\text{λ} max} {\text{-A}}_{410}\right) / \left(410 \text{-} \text{λ} \text{max}\right) \geq 0.005$$
in the expressions, A_{λmax} indicates a maximum absorbance at 300 to 400 nm.

2. The curable resin composition according to claim 1,
wherein the acidic group is selected from a carboxy group, a phosphono group, a phosphonooxy group, a hydrohydroxyphosphoryl group, a sulfino group, a sulfo group, or a sulfanyl group.

3. The curable resin composition according to claim 1,
wherein the acidic group is a carboxy group.

4. The curable resin composition according to claim 3,
wherein the polymer has, as a structural portion including the acidic group, a structural portion represented by General Formula (PA1) at one terminal of a polymer chain, 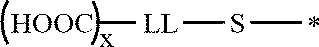
in the formula, LL represents a single bond or an (x+1)-valent linking group, where x represents an integer of 1 to 8, and * represents a bonding portion.

5. The curable resin composition according to claim 1,
wherein a weight-average molecular weight of the polymer is 1000 to 20000, andan acid value of the polymer is 2.0 mgKOH/g or more and less than 100 mgKOH/g.

6. The curable resin composition according to claim 1,
wherein L^{p} in General Formula (P) is a single bond, —CH₂—, —CH₂O—, or —CH₂CH₂O—.

7. The curable resin composition according to claim 1,
wherein a proportion of the constitutional unit represented by General Formula (P) to all constitutional units constituting the polymer is 10 mol% or more.

8. The curable resin composition according to claim 1,
wherein the near-ultraviolet light-absorbing organic compound is at least one of Compounds 1 to 3,
Compound 1: 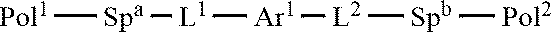
in the formula, Ar¹ represents an aromatic ring group represented by any of General Formula (2-1), ..., or (2-4),
L¹ and L² represent a single bond, —O—, —S—, —C(═O)—, —OC(═O)—, —C(═O)O—, —OC(═O)O—, —NR¹⁰¹C(═O)—, —C(═O)NR¹⁰²—, —OC(═O)NR¹⁰³—, —NR¹⁰⁴C(═O)O—, —SC(═O)—, or —C(═O)S—, R¹⁰¹ to R¹⁰⁴ represent -Sp^{c}-Pol³,
Sp^{a} represents a linking group having a shortest atom number of 2 or more and linking Pol¹ and L¹, Sp^{b} represents a linking group having the shortest atom number of 2 or more and linking Pol² and L², Sp^{c} represents a single bond or a divalent linking group, and
Pol¹ to Pol³ represent a hydrogen atom or a polymerizable group, in which at least one of Pol¹ or Pol² represents a polymerizable group, where a linking portion of Sp^{a} to L¹ and a linking portion of Sp^{b} to L² are both —CH₂—, and a linking portion of Sp^{a} to Pol¹, a linking portion of Sp^{b} to Pol², and a linking portion of Sp^{c} to Pol³ are all a carbon atom, 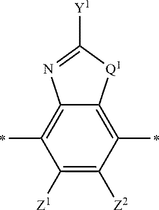 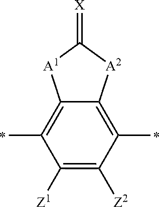 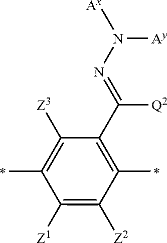 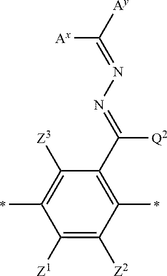
in the formulae, Q¹ represents —S—, —O—, or >NR¹¹, and R¹¹ represents a hydrogen atom or an alkyl group having 1 to 6 carbon atoms,
Y¹ represents an alkyl group having 1 to 6 carbon atoms, an aromatic hydrocarbon group having 6 to 12 carbon atoms, or an aromatic heterocyclic group having 3 to 12 carbon atoms,
Z¹, Z², and Z³ represent a hydrogen atom, an aliphatic hydrocarbon group having 1 to 20 carbon atoms, an alkoxy group having 1 to 20 carbon atoms, an alicyclic hydrocarbon group having 3 to 20 carbon atoms, an aromatic hydrocarbon group having 6 to 20 carbon atoms, a halogen atom, a cyano group, a nitro group, -NR¹²R¹³, or —SR¹², Z¹ and Z² may be bonded to each other to form an aromatic hydrocarbon ring or an aromatic heterocyclic ring,
R¹² and R¹³ represent a hydrogen atom or an alkyl group having 1 to 6 carbon atoms,
A¹ and A² represent —O—, >NR²¹, —S—, or >C(═O), and R²¹ represents a hydrogen atom or a substituent,
X represents ═O, ═S, a carbon atom to which a hydrogen atom or a substituent is bonded, or a nitrogen atom to which a hydrogen atom or a substituent is bonded,
A^{x} represents an organic group having 1 to 30 carbon atoms, which has at least one aromatic ring selected from an aromatic hydrocarbon ring or an aromatic heterocyclic ring, A^{y} represents a hydrogen atom, an alkyl group having 1 to 6 carbon atoms, or an organic group having 1 to 30 carbon atoms, which has at least one aromatic ring selected from an aromatic hydrocarbon ring or an aromatic heterocyclic ring, and A^{x} and A^{y} may be bonded to each other to form a ring,
Q² represents a hydrogen atom or an alkyl group having 1 to 6 carbon atoms, and
* represents a bonding position with L¹ or L²,
Compound 2: 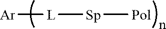
in the formula, Ar represents a group represented by General Formula (A1),
L represents a single bond, —O—, —S—, —C(═O)—, —OC(═O)—, —C(═O)O—, —OC(═O)O—, —NR³⁰¹C(═O)—, —C(═O)NR³⁰²—, —OC(═O)NR³⁰³—, —NR³⁰⁴C(═O)O—, —SC(═O)—, or —C(═O)S—,
R³⁰¹ to R³⁰⁴ represent —Sp^{d}—Pol⁴,
Sp and Sp^{d} represent a single bond or a divalent linking group, and Pol and Pol⁴ represent a hydrogen atom or a polymerizable group, and
n is an integer of 1 or 2,
where the compound represented by General Formula (A) has at least one polymerizable group, 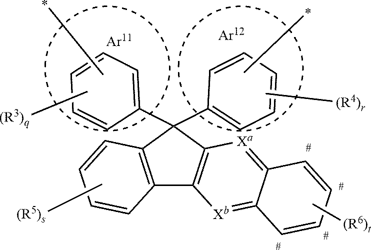
in the formula, Ar¹¹ and Ar¹² represent an aromatic hydrocarbon group including a benzene ring surrounded by a broken line or an aromatic heterocyclic group including a benzene ring surrounded by a broken line as one of rings constituting a fused ring,
X^{a} and X^{b} represent a nitrogen atom or CH, CH at a position of # may be substituted by a nitrogen atom,
R³ to R⁶ represent a substituent, q, r, s, and t are an integer of 0 to 4, and
* represents a bonding position with Pol—Sp—L—,
Compound 3: 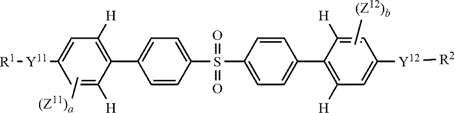
in the formula, a and b are an integer of 1 or 2, Y¹¹ and Y¹² represent —S— or —O—, R¹ and R² represent a hydrogen atom, a methyl group, or an ethyl group, and Z¹¹ and Z¹² represent a methyl group or an ethyl group having a substituent represented by General Formula (Z), 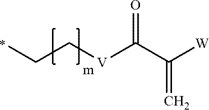
in the formula, m is an integer of 0 or 1, W represents a hydrogen atom or a methyl group, and V represents —O—CₙH₂ₙ—O—**, —S—CₙH₂ₙ—S—**, or —S—CₙH₂ₙ—O—**, where ** represents a bonding site with a (meth)acryloyl group, and n is an integer of 2 to 4, where at least one hydrogen atom in —CₙH₂ₙ— is substituted by a methyl group.

9. The curable resin composition according to claim 1,
wherein a content of the polymer is 5 to 50 parts by mass with respect to 100 parts by mass of a content of the indium tin oxide particles.

10. The curable resin composition according to claim 1,
wherein a content of the indium tin oxide particles in the curable resin composition is 10% to 60% by mass.

11. The curable resin composition according to claim 1,
wherein a particle diameter of the indium tin oxide particles is 5 to 50 nm.

12. The curable resin composition according to claim 1, further comprising:
a monofunctional or bi- or higher functional (meth)acrylate monomer compound.

13. The curable resin composition according to claim 1, further comprising:
a polymerization initiator.

14. A cured product of the curable resin composition according to claim 1.

15. A diffractive optical element comprising:
the cured product according to claim 14,
wherein the diffractive optical element includes a surface having a diffraction grating shape and formed of the cured product.

16. A multilayer diffractive optical element comprising:
a first diffractive optical element; and
a second diffractive optical element,
wherein the first diffractive optical element is the diffractive optical element according to claim 15, and
a surface of the first diffractive optical element, which has a diffraction grating shape, and a surface of the second diffractive optical element, which has a diffraction grating shape, face each other.
